(19) United States
(12) Patent Application Publication
(10) Pub. No.: US 2006/0160153 A1
(43) Pub. Date: Jul. 20, 2006

(54) Method to measure a t cell response and its uses to qualify antigen-presenting cells

(76) Inventors: Jean-Pierre Abastado, Paris (FR); Nadege Bercovici, Paris (FR); Marc S. Ernstoff, Us-Hanover, NH (US); Alice L. Givan, Us-Durham, NH (US); Alessandra Nardin, Paris (FR); Margarita Magguilli born Salcedo, Chatillon (FR); Paul K. Wallace, Orchard Park, NY (US)

Correspondence Address: YOUNG & THOMPSON, 745 SOUTH 23RD STREET, 2ND FLOOR, ARLINGTON, VA, 22202, US

(21) Appl. No.: 10/537,500

(22) PCT Filed: Dec. 2, 2003
(86) PCT No.: PCT/EP03/13579

§ 371 Date: Dec. 2, 2005

Related U.S. Application Data
(60) Provisional application No. 60/430,347, filed on Dec. 3, 2002.

(51) Int. Cl. G01N 33/567

(52) U.S. Cl. 435/7.21

(57) ABSTRACT

A method to characterize a T-cell response of a final population of T-lymphocytes resulting from the co-incubation of an initial population of T lymphocytes with a composition of antigen-presenting cells (APCs), said method comprising the steps of a) simultaneous measuring on a single cell basis at least two parameters: (i) proliferation of T lymphocytes and (ii) presence of a T cell antigen receptor on the surface of T lymphocytes and/or presence of at least one biological molecule produced by T lymphocytes, and the attribution of a positive or a negative value to each of these parameters, and b) classification of the final T-lymphocytes population into 2ⁿ different subsets of T lymphocytes, n being the number of parameters, each subset being characterized by a positive or a negative value respectively to each parameter and the determination of the proportion of T lymphocytes present in each subset with respect to the number of T lymphocytes in the final population, with said proportion being characteristic of the T-cell response. Use of the method for batch release assay and potency assay.

### FIELD OF THE INVENTION

The present invention relates to a new method to characterize a T-cell response of a final population of T lymphocytes resulting from the co-incubation of a composition of antigen-presenting cells (APCs) with an initial population of T lymphocytes. The present invention also relates to the use of the new method to qualify APCs.

### BACKGROUND AND PRIOR ART OF THE INVENTION

All the patent applications and articles are included herein for references.

Antigen-presenting cells (APCs) play a crucial role in controlling the initiation and orientation of Ag-specific immune responses. The influence of APCs maturation on T cell recruitment, activation, expansion and functional differentiation is currently widely investigated. A classical method to evaluate the capacity of APCs to recruit and expand T cells is the nixed lymphocytes reaction (MLR). This method rests upon the mixture in co-culture of T-cells and APCs originating from two different persons. The APCs differing from T cells in point of view of MHC-II, they induce activation of T lymphocytes and their proliferation, that may be measured by incorporating of [³H]-thymidine. In this case, the response obtained is not specific for a given antigen. Such a method may also be applied in a case where the two type of cells originate from the same person. In this situation, the APCs should be loaded with exogenous antigen. A major drawback of the method is that it gives information about a global population and may not help to distinguish the different subtypes of T cells that may respond differentially to a given stimulus resulting from co-incubation with APCs.

Antigens encountered by T cells affect their proliferation potential and drive acquisition of effector functions including cytokine synthesis and cytolytic activity as well as long term survival (Champagne et al., DNA Cell Biol 2001. 20: 745-760; Kaech et al. Nat Rev Immunol 2002. 2: 251-62; Lanzavecchia. and Sallusto Science 2000. 290: 92-7).

While the plasticity and diversity of T cell responses have been recognized for a long time, few quantitative studies have been conducted to measure what proportion of specific T cells will enter a given differentiation program after antigen stimulation.

Because of the small proportion of cells that respond to any specific antigen, describing the response to this antigen quantitatively is difficult.

Enumeration and characterization of antigen-specific T cells is limited by the low frequency of T lymphocytes, present in an initial population of non-stimulated cells that may respond to a given antigen (“precursors”), and also by the particular readout chosen to identify a T cell as specific for any particular antigen. A precursor is a T lymphocyte present in an initial population of non-stimulated T cells that may respond to a given antigen presented by APCs.

The difficulty in evaluating the repertoire of T cells, naive or experienced, that can potentially respond to a given antigen relates to the diversity of the T cell clones, to the low frequency of these clones, and to the pattern of effector functions shaped by previous antigenic challenge.

The generation of MHC/peptide tetrameric complexes (Altman et al. Science 1996. 274: 94-96), ELISPOT assays (Czerkinsky et al. 1983. 65 (1-2), 109-121; Herr et al. Immunol Methods 1996. 191, 131-42), intracellular or affinity matrix detection of cytokines (Jung et al. J Immunol Methods 1993. 159: 197-207; Mathioudakis et al. J Immunol Methods 2002. 260: 37-42; Pala et al. J Immunol Methods 2000. 243: 107-124, Manz et al. 1995. 92: 1921-1925) and more recently quantification with T-cell receptor (TCR) clonotypic probes (Lim et al. J Immunol Methods 2002. 261: 177-194) constitute reliable, sensitive approaches for the monitoring of antigen-specific T cells ex vivo (i.e., with limited or no in vitro culture). It does not adress their functional role nor potency.

MHC/peptide tetramers conjugated with fluorochromes allow the detection of epitope specific T cells based on single cell analysis by flow cytometry. Use of these tetramers has greatly contributed to our understanding of mature T cell differentiation during the immune response to pathogens or following vaccination (Klenerman et al. Nat Rev Immunol 2002. 2: 263-272; Murali-Krishna et al. Immunity 1998. 8: 177-187; Pittet et al. Int Immunopharmacol 2001. 1: 1235-1247). This monitoring has so far been essentially restricted to CD8⁺ T cells. MHC/peptide tetramers directed against CD4⁺ T cells are becoming however more widely available.

The capacity of cells to bind tetramers does not imply any particular effector function. For example, detection of anergic specific CD8⁺ T cells has been described in peripheral blood of patients (Lee et al. Nat Med 1999. 5: 677-685). The combination of tetramer staining with detection of intracellular cytokines or effector molecules such as perform produced in response to antigen-specific stimulation allows direct visualization of the pattern of cytokines produced by tetramer-binding cells (Appay and Rowland-Jones J Immunol Methods 2002. 268: 9).

However, as recognition of MHC/peptide complexes by the TCR of T cells is degenerated (Mason et al. Immunol Today 1998. 19: 395-404) the definition of antigen-specific T cells based simply on a stable interaction with these tetramers is questionable.

Proliferative potential itself constitutes an important parameter for evaluating the differentiation status of antigen-specific T cells. Naive T cells have the capacity to expand and give rise to effector/memory cells (Champagne et al. DNA Cell Biol 2001. 20: 745-760; Kaech et al. Nat Rev Immunol 2002. 2: 251-262; Lanzavecchia and Sallusto Science 2000. 290, 92-97). The T cells that will compose this pool are thought to acquire a high proliferative potential in order to mount a rapid secondary immune response. Other T cells are thought to progressively lose their capacity for clonal expansion after they have terminally differentiated into cells mediating cytokine secretion and killing activity (Champagne et al. Nature 2001. 410: 106-111; Sallusto et al. Nature 1999. 401: 708-712).

One method for assaying proliferation utilizes cell labeling with vital fluorescent dyes (Horan and Slezak Nature 1989. 340: 167-168; Lyons and Parish J Immunol Methods 1994. 171: 131-137; Allsopp et al. J Immunol Methods 1998. 214: 175-186; Lyons J Immunol Methods 2000. 243: 147-154; Wells, et al. J Clin Invest 1997. 100: 3173-3183). This assay has been used in various models to track cell division after stimulation either in vitro or, alternatively, in vivo following adoptive transfer (Wallace et al. Cancer Res 1993. 53: 2358-2367; Geginat et al. J Exp Med 2001. 194: 1711-1719; Champagne et al. Nature 2001. 410: 106-111; Gudmundsdottir et al. J Immunol 1999. 162: 5212-5223; Kaech and Ahmed Nat Immunol 2001. 2: 415-422: Kassiotis et al. Nat Immunol 2002. 3: 244-250; van Stipdonk et al. Nat Immunol 2001. 2: 423-429; Veiga-Fernandes, et al. Nat Immunol 2000. 1: 47-53). The substantial equal partition of these fluorescent dyes between daughter cells during cytokinesis allows the use of fluorescence intensity to visualize the successive generations of expanding cells and thus has contributed to a better definition of requirements for T cell expansion and survival.

Few groups, however, have taken advantage of the dye dilution to calculate back to the precursor frequency of the proliferating cells in the original T cell population (Wells, et al. J Clin Invest 1997. 100: 3173-3183; Givan et al. J Immunol Methods, 1999. 230: 99-112; Song et al. J Immunol 1999. 162, 2467-2471; Gett and Hodgkin, Nat Immunol 2000. 1 (3):239-244). Indeed, because of the exponential expansion of specific T cells, observation of cells without tracking molecules by flow cytometry after several days of culture is misleading.

The presents inventors have previously described a flow dye dilution assay to calculate the precursor frequency and expansion potential of antigen-specific T cells (Givan et al. J Immunol Methods, 1999. 230: 99-112). Precursor frequencies of cells proliferating in response to tetanus toxoid antigen calculated by this dye dilution assay correlated well with, but were about 100-fold higher than, results obtained by the traditional limiting dilution analysis (LDA) using tritiated thymidine.

In some models, however, clonal expansion has been shown to tightly regulate the production of cytokines (Bird et al. Immunity 1998. 9: 229-237; Gett and Hogkin Proc Natl Acad Sci USA 1998. 95: 9488-9493; Gudmundsdottir et al. J Immunol 1999. 162: 5212-5223) suggesting that the time of stimulation is critical.

The inventors developed a new method to characterize a T-cell response of a final population of T lymphocytes resulting from the co-incubation of a composition of antigen-presenting cells (APCs) with an initial population of T lymphocytes. The method is based on a multiparameter flow cytometric method which allows, on a single cell basis, the simultaneous analysis of at least two parameters, one being the T-cells proliferating and the other detection of presence of T cell antigen receptor and/or detection of presence of at least one biological molecules produced by T lymphocytes. This method may be extended to the detection of presence of at least one surface determinant markers, different from the T cell antigen receptor.

APCs may be co-incubated with an initial population of T-cells without prior loading with exogenous antigen or antigens in order to characterize a T-cell response of final population related to autoantigen or autoantigens present in APCs before their isolation from a mammal or a human.

APCs may be loaded, after their isolation from an animal or a human, with an antigen or a fragment of an antigen or a mixture of antigens (or fragments of antigens) or with a vector containing a gene encoding for an antigen prior to co-incubation with an initial population of T-cells in order to characterize a T-cell response of a final population related to the antigen or antigens loaded in the APCs.

This method allows classification of a population of T-lymphocytes into 2ⁿ subsets, n being the number of parameters chosen for the analysis, that is to say n≧2.

This new method also comprises a new determination method to evaluate the relative precursor frequencies of sub-populations (or subsets) with different potential responses within a mixed population of cells.

The present invention also relates to the use of a method such as described above, as a potency assay of an ex-vivo composition of APCs.

The present invention also relates to the use of a method such as described above, as a method to evaluate an effect of one or more cytokines produced by a composition of APCs on a T-cells response.

The present invention also relates to the use of a method such as described above, as a method to evaluate an effect of one or more surface determinant markers present on T-cells on a T-cell response resulting from their co-incubation with a composition of APCs.

The present invention also relates to the use of a method such as described above, as a batch release assay for an ex-vivo composition of APCs.

The present invention also relates to the use of a method such as described above, as an inclusion criteria for a patient.

The present invention also relates to the use of a method such as described above, as an antigen selecting assay.

The present invention also relates to the use of a method such as described above, as an assay to detect the presence of pathogenic T lymphocytes present in a patient. T lymphocytes are considered as pathogenic when they induce an autoimmune reaction.

The present invention also relates to the use of a method such as described above, to define standard control T cell response of T lymphocytes.

The present invention also relates to the use of a method such as described above, as an assay to evaluate the efficiency of a process to load an antigen into an antigen-presenting cell.

The present invention also relates to the use of a method such as described above, as an assay to qualify an antigen batch.

The present invention also relates to the use of a method such as described above, as an assay to evaluate the impact of a method of antigen preparation on the ability of antigen-presenting cell to present antigen.

The present invention also relates to the use of a method such as described above, as an assay to evaluate the stability of a presentation of an antigen by APCs.

The biological molecules may reflect cytokine production (IFN-γ, IL-4, IL-5, IL-10), enzyme production (granzyme, perforine), or chemokine production. The surface determinants markers may be markers of T cells activation such as CD25 and CD69, markers of T cell differentiation or migration such as CD27, CD28, CD62L and CCR7.

This new method presents the advantage of integrating in a single assay and on a single cell basis, the means to examine the complexity relating to the diversity of T cell clones, to the low frequency of these clones, and to the pattern of effector functions shaped by previous antigenic challenge, in order to describe the diversity of a specific T-cell pool.

One advantage of the method is that it allows the determination of how quantification of antigen-specific T cells by functional assays (cytokine synthesis or proliferation) relates to enumeration of epitope-specific T cells with tetramers of MHC/peptide.

An advantage of the invention is that it allows identification of different T lymphocytes subsets.

Another advantage of the method is that it allows for an estimate of the precursor proportion of each functional subset of T lymphocytes, defined by the parameters used in the measurement (such as proliferation, T cell antigen receptor, cytokine secretion) within the initial population. It could be applied to additional markers of function and differentiation (such as determinant surface markers different from T cell antigen receptor, enzymes secretion, chemokines secretion), combining all those parameters into a description of the complex response potential of a T-cell pool.

One advantage of the invention is that it benefits from sub-population expansion to increase the sensitivity for detecting rare responsive cells and for calculating the precursor frequencies of sub-population in the original mixture of cells.

For example, this new method allows detection of some rare precursors being able to produce cytokine such as IFN-γ but that do not expand. These results indicate that some CD8⁺ T cells do not require clonal expansion in vitro to produce cytokine such as IFN-γ. Thus, the new method could be used to compare the frequency of these precursors in various populations of effector/memory T cells (Sallusto et al. 1999 Nature. 401:708-712).

An advantage is that, because the method involves the calculation of precursors frequencies, the method is not biased by the length of culture time and by the expansions of certain cell population.

Another advantage of the invention is that it allows to describe an original population of resting T lymphocytes or precursors (before culture) in terms of its ability to react in different ways to antigen stimulation. This in turn could be used to characterize a composition of APCs loaded with an antigen, or a fragment of antigen, in term of capacity of the APCs to activate a particular subset of T lymphocytes. The method measures the effectiveness of the complex cross-talk from APCs to T lymphocytes and from T lymphocytes to APCs when a specific antigen is presented or a particular APC is used.

### DETAILED DESCRIPTION OF THE INVENTION

Abbreviations
APC: antigen-presenting cell
CMV: cytomegalovirus
DC: dendritic cell
DMSO: dimethyl sulfoxyde
EBV: Epstein-Barr Virus
E/T: effector/target
HD: healthy donor
IFN-γ: interferon-γ
LN: lymph node
mAb: monoclonal antibody
MHC: major histocompatibility complex
PBMC: peripheral blood mononuclear cells
PF: precursor frequency
PI: proliferation index
PMD: precursor mean division
SFC: spot forming cell
SN: supernatant
T_{CM}: central-memory T lymphocytes
TCR or TcR: T cell receptor
T_{EM}: effector-memory T lymphocytes

The present invention relates to a method to characterize a T-cell response of a final population of T lymphocytes resulting from the co-incubation of a composition of antigen-presenting cells (APCs) with an initial population of T lymphocytes.

This method comprises two steps that are:
(1) simultaneous measure, on a single cell basis, of at least two parameters the first parameter being necessarily proliferation of T lymphocytes and the second parameter being necessarily chosen among the group consisting of presence of T cell antigen receptor on the surface of T lymphocytes and presence of at least one biological molecule produced by T lymphocytes, chosen among n parameters, n≧2, and the attribution of a positive or a negative value to each of these parameters,
(2) classification of the final T-lymphocytes population into 2ⁿ different subsets of T lymphocytes, n being the number of parameters, each subset being characterized by a positive or a negative value respectively to each parameter, and the determination of the proportion of T lymphocytes present in each subset with respect to the number of T lymphocytes in the final population, with said proportion being characteristic of the T-cell response.

The present invention also relates to a method in which step (1) is extended to comprise in addition to the above measured parameters, the measure of an additional parameter being at least one surface determinant marker on T lymphocytes, different from T cell antigen receptors.

The terms “initial population of T lymphocytes” mean any population of T lymphocytes that was not submitted to a co-incubation with antigen-presenting cells for the purpose of the present invention. However, that does not exclude that before isolating T lymphocytes for use according to the invention, those cells had been in contact in vivo or in vitro with APCs. The T lymphocytes may be obtained from any animal or human, healthy or a patient. The T lymphocytes may come from peripheral blood or from a biopsy from tumor (tumor infiltrating lymphocytes) or tumor-invaded lymph node or any suitable tissue. When coming from blood, the T lymphocytes may be obtained by any technique known by the man skilled in the art of taking a blood sample. A technique that allows taking a blood sample is for example aphaeresis (or apheresis or cytapheresis). An apheresis is any procedure in which blood is drawn from a donor or patient and a component (platelets, plasma, or white blood cells) is separated out, the remaining blood components being returned to the body. The T lymphocytes may also be a cell line or a clone specific for a given antigen. It cannot be excluded that T lymphocytes taken from an animal or a human may have been in contact with antigen-presenting cells in vivo. But such cells should be considered as “initial population” because such contact was not intended for the purpose of the invention. A cell line is a population of cells of plant or animal origin capable of dividing indefinitely in culture.

The terms “final population of T lymphocytes” mean any population of T lymphocytes obtained after a contact with antigen-presenting cells for the purpose of the invention.

The term “proliferation” means an increase in the number of cells as a result of cell division. For the purpose of invention, the cells are considered as being divided when they divided at least once.

The term “parameter” means that which allows to characterize a cell type. Parameters characterizing a cell type may be altered by stimuli affecting the cells. Non limited examples of parameters that may be used to characterize a cell type are: presence of specific intracellular biological molecule (enzyme or structure protein); presence of membrane biological molecule (receptor, protein of attachment, enzyme); secretion of a biological molecule (cytokine, enzyme); presence or absence of intracellular organelles, number of nucleus. Parameters may also be characteristic of the status of a cell such as growth, division, apoptosis, necrosis. Parameters may also be characteristics of cell functionality. All these type of parameters are well known from the man skilled in the art. Those parameters may be detected with numerous chemical-based, colorimetric-based, electrophysiology-based, radioactive-based or fluorescent-based methods known from the man skilled in the art and adapted to the parameter to be detected and measured. Two parameters allow to characterize T lymphocytes, which are the number of divisions of T lymphocytes induced by the contact with APCs and the T cell receptor.

The terms “surface determinant marker” means any molecule characteristic of the plasma membrane of a cell or in some cases of a specific cell type

The terms “T cell response” mean the cellular events that follow activation of T lymphocytes after, for example, incubation with APCs and that may result in, for example, cell proliferation, secretion of cytokines, down- or up-regulation of expression of surface or intracellular determinant markers.

The terms “T cell antigen receptor” (or T cell receptor or TCR or TcR) mean antigen receptor expressed by T cells and used in the detection of antigen. Those receptors are made of α-, β- or γ- and δ-chains that, diversely matched, allowing the T lymphocytes to recognize antigens in the MHC framework. With the TcR, T lymphocytes may recognize antigenic peptides combined with MHC I or MHC II molecules. Considered as being an antigen, is any substance liable to bind specifically to antibody. However, some antigens do not, by themselves, elicit antibody production.

According to the present invention, antigen-presenting cells and T lymphocytes may be autologous or allogeneic, that is to say isolated from the same human or the same animal or isolated from a different individual or syngeneic animal.

According to the invention, the initial population of T lymphocytes should be understood as a population of cells that has been isolated from a mammal or a human. The final population of T lymphocytes should be understood as a population of cells obtained following the co-incubation with a composition of APCs for the purpose of the invention.

In some cases, sub-populations (or subsets) of T-cells of an animal or a human may recognize some self-antigens presented by the cells of this animal or this human and react against those cells to destroy them. A self-antigen is an antigen of one's own cells or cell products. This may lead to autoimmune disease. Those self-antigens may be presented by cells belonging to any tissue or organ or by some APCs.

In a particular mode of realization of the present invention, APCs may be co-incubated with an initial population of T-cells without prior loading with exogenous antigen or antigens in order to characterize a T-cell response of final population. The T-cells response of the final population is related to the autoantigen or autoantigens that were present in APCs before their isolation from a mammal or a human.

In an other particular embodiment of the present invention, APCs may be loaded with an antigen or a fragment of an antigen or a mixture of antigens (or fragments of antigens) or with a vector containing a gene encoding for an antigen, prior to co-incubation with an initial population of T-cells in order to characterize a T-cell response to the antigen or antigens used with the APCs.

It should be understood that according to the invention the terms “biological molecule produced by T cells” mean any molecule (proteins, peptides, lipids, glycolipids, glycosyl derivatives or any second messengers resulting from activation of any signal transduction pathway that is known by the skilled person in the art) that can be produced by a T cell but does not encompass surface determinant markers. The detection of biological molecule aims to functionally characterize a given population of T cells. Biological molecules the presence of which are detected in final population of T lymphocytes, are for example cytokines or chemokines or enzymes. Cytokines the presence of which may be detected are for example IFN-γ, IL-2, IL-4, IL-5, IL-10. Chemokines, the presence of which may be detected, are for example ligand for CCR5, CCR7. Enzymes, the presence of which may be detected, are for example perforine or granzyme. A surface determinant marker means a molecule that is expressed at the surface of a cell and its presence, alone or in combination with other surface determinant markers, is associated with a phenotype of a given population of T cells. Surface determinants markers the presence of which may be detected are for example CD4, CD8, CD25, CD28, CD69, CTLA-4, CD45-RA, CD45-RO, CD62-L. Intracellular detection of cytokines by flow cytometry may be based on direct detection of intracellular cytokine expression with fluorochrome-conjugated antibodies after period of activation with various stimuli. Cells are stimulated before the measure a sufficient time allowing the production of the cytokines to be measured. Cytokines secretion is disrupted during the latter portion of the incubation with the addition of drugs that inhibit cytokines secretion such as monensin or brefeldin A, allowing the accumulation of cytokines inside the cells. Cells are then fixed using paraformaldehyde or similar agents. Permeabilization of cell membrane is achieved using nonionic detergents or alcohol, followed by intracellular staining using mixtures of fluorescent labeled-antibodies that recognize determinants in fixed and permeabilized cells or their corresponding isotype controls. Unstimulated leukocytes do not express or express very low level of cytokine. Because background constitutive cytokine expression is rare, very low frequencies of positive stimulated cells can be detected. A classical process of detection of biological molecules, such as cytokines as described above, is usable for the others biological molecules that are chemokines and enzymes. An other mean to detect cytokines may be based on a procedure previously described by Manz et al. using bispecific antibodies (Manz et al. Proc Natl Acad Sci USA 1995. 92: 1921-1925). Bispecific antibodies have one site of recognition which is directed toward a cytokine to be trapped, the other site is directed toward a surface determinant marker such as CD45 present on surface of T cells. They are added to T lymphocytes before their incubation with APCs in order to be linked to T cells before cytokines are secreted out of cells. Such method allows to detect cytokines on the surface of living cells, and to sort the cells according to the presence of cytokines to be detected.

Surface determinant markers are usually detected with fluorochromes-conjugated antibodies directed against a specific epitope of the markers.

The simultaneous measurement on a single cell basis of at least two parameters as defined above can be made using a flow cytometry apparatus known from the man skilled in ,the art. The flow cytometry system allows simultaneous detection and measurement on a single cell, by the way of fluorescence detection, multiple parameters, provided that the different fluorochromes used to characterize each parameter are different ones from each others and that their emission spectra do not overlap.

The attribution of positive or a negative value to a given parameter relies upon the variation between the fluorescence measured in control cells (or sample) and fluorescence measured in tested cells (or sample). When the variation is at least equal or greater than a given multiple of standard deviation of the mean fluorescence determined for the control cells (or sample), a positive value is attributed to the given parameter. The given multiple is determined by the way, of routine experiments known from the man skilled in the art for each parameter. The control may be internal to the sample to be tested. For instance, in an experiment measuring proliferation and presence of a given T cell antigen receptor, the internal control is represented by the cells that do not proliferate and that do not present the given T cell antigen receptor.

The attribution of positive or negative value to a given parameter may be achieved with others methods, well-known by the man skilled in the art. For example, according to one method, the cells may be considered positive when greater in fluorescence intensity than 98% of the negative cells.

When measuring the fluorescence intensity of a given parameter from a cell population, the result may be given under the form of an histogram wherein the X-axis corresponds to the fluorescence intensity of the measured parameter and the Y-axis corresponds to the number of cells. From such histogram it may be determined a fluorescence intensity under which there are 98% of the cell population.

In this method, the threshold above which the cells from a sample to be tested may be considered as being positive is the value of fluorescence under which 98% of the cells from the control sample are fluorescent.

The attribution of a positive or a negative value for each chosen parameter may be used to define some subsets of T lymphocytes in the final population. The number of subsets observed in the final population is dependent on the number (n) of parameters chosen according to the formula 2ⁿ. When two parameters are used, the different subsets are determined according to the capacity (or not) of T lymphocytes to proliferate (P⁺ or P⁻) and to the presence (or not) of the specific T cell antigen receptor (TCR⁺ or TCR⁻) or the presence (or not) of at least one biologicals molecules (C⁻, C⁺). Using this method, the T lymphocytes may be classified in four different subsets (P⁻, TCR⁻), (P⁺, TCR⁻), (P⁻, TCR⁺) and (P⁺, TCR⁺) or (P⁻, C⁻), (P⁺, C⁻), (P⁻, C⁺) and (P⁺, C⁺).

When more than two parameters are used according to the present described method, those additional parameters are used to more accurately define the different T lymphocytes subsets and determine their proportion in the sample after the incubation in presence of APCs.

For example when the presence (or absence) of a surface determinant marker (D), other than T cell receptor antigen, is determined, the different subsets of T cells may be defined as follows: (P⁻, TCR⁻, D⁻), (P⁺, TCR⁻, D⁻), (P⁻, TCR⁺, D⁻), (P⁻, TCR⁻, D⁺), (P⁺, TCR⁺, D⁻), (P⁺, TCR⁻, D⁺), (P⁻, TCR⁺, D⁺), (P⁺, TCR⁺, D⁺).

For example when the variation of level of biological molecules (C) is determined, the different subsets of T cells may be defined as follows: (P⁻, TCR⁻, C⁻), (P⁺, TCR⁻, C⁻), (P⁻, TCR⁺, C⁻), (P⁻, TCR⁻, C⁺), (P⁺, TCR⁺, C⁻), (P⁺, TCR⁻, C⁺), (P⁻, TCR⁺, C⁺), (P⁺, TCR⁺, C⁺).

For example when the presence (or absence) of a surface determinant marker (D) and the variation of level of biological molecules (C) are determined, the different subsets of T cells may be defined as follows: (P⁻, TCR⁻, D⁻, C⁻), (P⁺, TCR⁻, D⁻, C⁻), (P⁻, TCR⁺, D⁻, C⁻), (P⁻, TCR⁻, D⁺, C⁻), (P⁻, TCR⁻, D⁻, C⁺), (P⁺, TCR⁺, D⁻, C⁻), (P⁺, TCR⁻, D⁺, C⁻), (P⁺, TCR⁻, D⁻, C⁺), (P⁻, TCR⁺, D⁺, C⁻), (P⁻, TCR⁺, D⁻, C⁺), (P⁻, TCR⁻, D⁺, C⁺), (P⁺, TCR⁺, D⁺, C⁻), (P⁺, TCR⁺, D⁻, C⁺), (P⁺, TCR⁻, D⁺, C⁺), (P⁻, TCR⁺, D⁺, C⁺), (P⁺, TCR⁺, D⁺, C⁺).

The measured parameters may also be one or more parameters of a given class such as one or more biological molecules produced by T lymphocytes (C₀ to Cₙ) or the combination of one or more surface determinants markers (D₀ to Dₙ). Multiple parameters of a given class may also be combined with multiple parameters of an other class. The potentiality of the present method being only limited by the number of channels available on a flow cytometry apparatus.

The determination of different subsets according to the number and the type of parameters chosen allows classification of the proportion of the different responsive (or not responsive) T cells in the final population.

According to a particular mode of realization of the present invention, the new method allows the determination from the proportion of T lymphocytes in each of the different subset present in the final population, of the proportion of T lymphocytes or their potential in each corresponding subset present in the initial population with respect to the number of T lymphocytes in the initial population.

A precursor is a cell that after exposure to a given stimulation evolves to give a cell presenting specific characteristics (surface determinant, cytokine secretion, proliferation capacity) but that initially did not express those specific characteristics. That means that a precursor is a cell that may potentially develop some characteristics depending on the stimuli that it receives. Therefore, the composition of a final population of T lymphocytes is dependent on the type and number of the precursors present in the initial population and of the nature of stimulus. Hence, by analyzing the proportion of T lymphocytes in the different subsets identified in the final population, the present method allows to determine the proportion of T cells in the initial population that are responsible for the proportion and distribution of T cells in the final population. Hence, although the T lymphocytes present in the initial population (or precursors) do not possess the characteristics of T lymphocytes present in final population (or dividing cells), they are classed in the subsets to which belong their heirs. The proportion of precursors (or precursors frequencies, PF) indicates the proportion of a given subset of T lymphocytes present in the initial population of T lymphocytes that may give a given response following a given stimulus.

The method that allows to determine, from the different proportions of T cells in the different subsets in the final population of T lymphocytes, the different proportions of T cells present in the initial population of T lymphocytes in the corresponding subsets (or proportion of precursors or precursors frequency) rests upon the following the step:
(i) determining intensity of fluorescence of non-proliferating, cells by analysis of either a sample not submitted to a proliferation stimulus or non-proliferating cells from the tested sample. When non-proliferating cells from the tested sample are used as internal control (that is to say cells that have not proliferated and that are present in the sample at the end of the experiment), the evaluation of their fluorescence is made during step (iii) (see below). Because some dye is lost from the cell membrane after a long period of culture (such as ten days), the width of the intensity of histogram of cells that have not proliferated and that are present in the sample at the end of the experiment may spread slightly with time.
(ii) gating cells according to parameters of interest defining a T lymphocytes subset,
(iii) examining fluorescence of probe used to measure proliferation of the gated cells,
(iv) deriving Gaussian curves centered on halving intensity values from the intensity of cells that have not proliferated and that are present in the sample at the end of the experiment (or cells from a control sample not submitted to a proliferation stimulus) to obtain Aₖ which is the proportion of cells in division k at the time of the assay. In order to exclude from the determination of specific proliferating cells those that may be classified as having undergone 0-1 division either because of the “artifactual” width of the parental generation or because of slow proliferation after long culture periods, cells that are considered to be proliferating for the purpose of data analysis are only those cells that had undergone two or more cell divisions.
(v) determining the proportion of cells (PF=precursor frequency) in the initial population of T lymphocytes (before stimulation) that have proliferated in order to give the proportion of cells present in the selected subset (step ii) using the formula:

$$PF = ( \begin{matrix}k = n \\ \sum {A}_{k} / {2}^{k} \\ k = 2\end{matrix} ) / ( \begin{matrix}k = n \\ \sum {A}_{k} / {2}^{k} \\ k = 0\end{matrix} )$$

wherein PF is precursor frequency in the initial population, Aₖ is the proportion of cells in division k at the time of the assay, k=0 for initial population of T lymphocytes, and cells having undergone 2 to n divisions having been classified as proliferating cells,
(vi) determining the percentage of non-proliferating cells from the percentage of cells that have not proliferated and that are present in the sample at the end of the experiment and half the percentage of cells that had undergone only one cell division. T cells that have divided only once are considered as having divided non specifically throughout the experiment,
(vii) applying the percent of non-proliferating cells to the number of gated cells in the data file to give the absolute number of cells in the corresponding subset before culture that will not proliferate according to the formula,
   number_{non-proliferating cells in the initial population}=[(proportion_{cells that leave not proliferated and that are present in the sample at the end of the experiment})+(0.5*proportion_{cells that have divided only once and that are present in the sample at the end of the experiment})]*[number_{gated cells in data file}]),
(viii) determining the absolute number of cells in the corresponding subset destined to divide by knowing the number of cells that was not destined to divide and the number of precursors cells of proliferating cells according to the formula,
   number_{proliferating cells in the initial population}=[(PF_{proliferating cells})*(number_{non-proliferating cells in the initial population})]/[1−PF_{proliferating cells}],
(ix) reiterating step (i) to step (vi) to each T lymphocytes subsets determined according to the n parameters used for the measure,
(x) summation of number of cells in the 2ⁿ subsets in order to express the number of cells present in the initial population as percent of the total original resting population.

In the method described above, steps (i) to step (iii) are made by using any flow cytometry apparatus known from the man skilled in the art and steps (iv) to (vi) are easily made in using ModFit software version 3.1 (Verity Software House, Topsham, Me.). The present part of the invention will be more clearly presented in the preferred embodiment section.

It should be noted that in step (iv) and (v), the generation 1 has been excluded from the calculation in order to reduce the artefact caused by the width of the parental generation or the slow proliferation occurring after long culture periods.

However, in some cases it may be useful to include the first generation of dividing cells in the calculation. It may be useful to include the first generation of dividing cells in cases where the contact between APCs and the initial population of T lymphocytes results in a rapid proliferation.

Such cases occur when, for example, cell stimulation calls upon phytohemagglutinin A or Concanavalin A or antibody anti-CD3.

In such cases the formula to be used at step (v) will be as follows:

$$PF = ( \begin{matrix}k = n \\ \sum {A}_{k} / {2}^{k} \\ k = 1\end{matrix} ) / ( \begin{matrix}k = n \\ \sum {A}_{k} / {2}^{k} \\ k = 0\end{matrix} )$$

wherein PF is precursor frequency in the initial population, Aₖ is the proportion of cells in division k at the time of the assay, k=0 for initial population of T lymphocytes, and cells having undergone 1 to n divisions having been classified as proliferating cells

And in step (vii), the formula to apply will be as follows:
number_{non-proliferating cells in the initial population}=[proportion_{cells that have not proliferated and that are present in the sample at the end of the experiment}]*[number_{gated cells in data file}],

The man skilled in the art will know from its experience when to include or exclude the first generation of dividing cells from the calculation.

In another aspect of the present invention, the new method allows the determination from the proportion of T lymphocytes in each of the different subset present in the final population, of the proportion of T lymphocytes or their potential in each corresponding subset present in the initial population with respect to the number of T lymphocytes in the initial population. The method of determination of the proportion of T-lymphocytes present in the initial population loaded with a fluorescent probe allowing the measure of proliferation is carried out according to the following the step:
(i) marking n minus 1 parameters, the parameter corresponding to the proliferation being previously marked, with fluorescent probes specific for each of the n minus 1 parameters,
(ii) gating T-lymphocytes in the final population of T lymphocytes according to the fluorescence of the n minus 1 chosen parameters, the measure of proliferation being excluded at this step, the value of which define lymphocytes subsets of interest,
(iii) building a fluorescent curve by recording the fluorescence intensity of the probe used to measure proliferation of the T-lymphocytes gated at step (ii),
(iv) possibly building a fluorescent curve by recording the fluorescence intensity of the probe used to measure proliferation from either:
   (iva) T-lymphocytes present in a lymphocytes subset defined by gating lymphocytes in the final population of T lymphocytes according to the absence of fluorescence of the n minus 1 chosen parameters or,
   (ivb) T-lymphocytes present in a sample of T-lymphocytes of the initial population not co-incubated with APCs,
(v) determining intensity of fluorescence of non-proliferating lymphocytes by analyzing the distribution of fluorescence of the fluorescent curve built at step (iii), or possibly at step (iv), the non-proliferating lymphocytes corresponding to the maximal value of fluorescence,
(vi) deriving, from the fluorescence curve recorded at step (iii), Gaussian curves centered on successive half intensity values derived from the maximal intensity of fluorescence determined from non-proliferating T-lymphocytes at step (iii) or at step (iv), to obtain Aₖ which is the proportion of cells in division k at the time of the measure of the proliferation,
(vii) determining the proportion of T-lymphocytes (PF=precursor frequency) in the initial population that have proliferated in order to give the proportion of T lymphocytes present in the selected subset (step ii) using the formula:

$$PF = ( \begin{matrix}k = n \\ \sum {A}_{k} / {2}^{k} \\ k = 2\end{matrix} ) / ( \begin{matrix}k = n \\ \sum {A}_{k} / {2}^{k} \\ k = 0\end{matrix} )$$

wherein PF is precursor frequency in the initial population, Aₖ is the proportion of cells in division k at the time of the measure of the proliferation k=0 for initial population of T lymphocytes, and cells having undergone 2 to n divisions having been classified as proliferating T lymphocytes,
(viii) determining the percentage of non-proliferating T-lymphocytes from the percentage of T-lymphocytes that have not proliferated and that are present in the final population of T-lymphocytes and half the percentage of T-lymphocytes that had undergone only one cell division,
(ix) applying the percent of non-proliferating T-lymphocytes to the number of gated T-lymphocytes in the data file to give the absolute number of T-lymphocytes in the corresponding subset before culture that will not proliferate according to the formula,
   number_{non-proliferating cells in the initial population}=[(proportion_{cells that have not proliferated and that are present in the sample at the end of the experiment})+(0.5*proportion_{cells that have divided only once and that are present in the sample at the end of the experiment})]*[number_{gated cells in data file}],
(x) determining the absolute number of T-lymphocytes in the corresponding subset destined to divide by knowing the number of T-lymphocytes that was not destined to divide and the number of precursor cells of proliferating T-lymphocytes according to the formula,
   number_{proliferating cells in the initial population}=[(PF_{proliferating cells})*(number_{non-proliferating cells in the initial population})]/[1−PF_{proliferating cells}],
(xi) reiterating step (i) to step (viii) to each T lymphocytes subsets determined according to the n parameters used for the measure,
(xii) summation of number of cells in the 2ⁿ subsets in order to express the number of T-lymphocytes present in the initial population of T-lymphocytes as a percentage of the total initial population before co-incubation.

The fluorescent probe used to mark the chosen parameters at step (i) may be a fluorescent antibody that binds directly to the parameter to be measured (such as biological molecules, determinant surface markers, . . . ) or indirectly via a first antibody which binds primarily to the parameter. According to the above-defined method the T-cell response of the final population of T-lymphocytes is characterized by measuring n parameters (n being an integral number designing the total number of parameters measured), one of them being necessarily the proliferation. The proliferation is measured by using a fluorescent probe loaded into the T-cells in the initial population of T-lymphocytes. Therefore, at the time of the proliferation measurement of the final population of T-lymphocytes, there are n minus 1 parameters left to be marked at the first step of the above-described method (step i).

The distribution of the fluorescence, on a linear scale (for example between 0 to 255), of the curve recorded at step (iii) is indicative of the proliferation of the T-cells. As the quantity of the fluorescent probe used to follow the proliferation is approximately halved during each division of the cell, the cells displaying a decreasing value of fluorescence compared to the maximal value of fluorescence correspond to the cells having divided. The cells displaying the maximal value of fluorescence are considered as being the non-proliferating cells. The maximal value of fluorescence is visually determined by the man skilled, according to the distribution of the cells along the curve of fluorescence recorded at step (iii).

However for such determination, a minimum number of events (cells displaying the maximal value of fluorescence namely non-proliferating cells) are required. For example, it may be required that at least 50 events, more preferably at least 100, more preferably at least 500, more preferably at least 5000, have to be distributed around the maximal value of fluorescence that allows the determination of the fluorescence of the non-proliferating cells. The term “around” should be understood as meaning that the fluorescence of the cells considered as being non-proliferating have not to differ from about 70%, from about 60%, from about 50%, from about 25%, more preferably from about 20%, more preferably from about 10%, more preferably from about 5%, more preferably from about 2%, from the value of fluorescence considered as being maximal. In some cases, the number of non-proliferating cells in the subset gated in step (ii) is not sufficient to determine visually the value of fluorescence of non-proliferating cells. A population of primary T-cells (directly taken from the blood of an animal, for example) that may have been previously in contact with the antigen used to load the antigen presenting cells may contain a great number, at least about 0.001%, at least about 0.01%, at least about 0.02%, at least about 0.1%, at least about 0.2%, at least about 0.5%, at least about 1%, at least about 5%, at least about 10%, at least about 20%, at least about 30%, at least about 40%, at least about 50%, at least about 60%, at least about 70%, at least about 80%, at least about 90% of T-cells able to respond to the contact with the antigen-presenting cells. Those cells may highly proliferate, namely divide at least once, more preferably at least twice, at least 3 times, at least 4 times, at least 5 times, at least 6 times, at least 7 times, at least 8 times, at least 9 times, at least 10 times, at least 15 times, at least 20 times, after contact with the antigen-presenting cells and the number of non-proliferating cells, in the subset gated at step (ii), will be insufficient to determine the value of fluorescence corresponding to the non-proliferating cells. In such case it may be possible to determine the maximal value of fluorescence in another subset of T-lymphocytes present in the final population but from which it may be known a priori that there are few proliferating cells, for example less than about 50%, less than about 40%, less than about 30%, less than about 20%, less than about 10%, less than about 5%, less than about 2%, less than about 1% in respect with the total number of cells. Such subset of T-cells may be a subset in which the T-cells are negative for the n minus 1 chosen parameters (step iva). The T-cells negative for the n minus 1 chosen parameter are likely to be the cells that do not respond to the stimulus represented by the antigen-presenting cells and therefore the cells that do not proliferate, Therefore the determination of the intensity of fluorescence of non-proliferating lymphocytes (step v) will be carried out by analyzing the fluorescence of the fluorescent probe used to measure the proliferation in a subset of T-cells negative for the n minus 1 parameters (step iva).

Another example where the number of non-proliferating cells may be insufficient to determine the value of fluorescence corresponding to the non-proliferating cells is when the initial population of T-lymphocytes is constituted by a clone population or a cell line. In such case, virtually all the cells have the ability to proliferate and therefore there will likely be substantially no non-proliferating cells, for example less than about 50%, less than about 40%, less than about 30%, less than about 20%, less than about 10%, less than about 5%, less than about 2%, less than about 1% in respect with the total number of cells. Therefore the determination of the intensity of fluorescence of non-proliferating lymphocytes (step v) will be carried out by analyzing the fluorescence of the fluorescent probe used to measure the proliferation in a sample non-submitted to the contact with the antigen-presenting cells loaded with an antigen (step ivb). In another aspect of the invention the method that allows to determine, from the different proportions of T cells in the different subsets in the final population of T lymphocytes, the different proportions of T cells present in the initial population of T lymphocytes in the corresponding subsets (or proportion of precursors or precursors frequency) rests upon the following the step:
(i) marking n minus 1 parameters, the parameter corresponding to the proliferation being previously marked, with fluorescent probes specific for each of the n minus 1 parameters,
(ii) gating T-lymphocytes in the final population of T lymphocytes according to the fluorescence of the n minus 1 chosen parameters, the measure of proliferation being excluded at this step, the value of which define lymphocytes subsets of interest,
(iii) building a fluorescent curve by recording the fluorescence intensity of the probe used to measure proliferation from either:
   (iiia) T-lymphocytes gated at step (ii) or,
   (iiib) T-lymphocytes present in a lymphocytes subset defined by gating lymphocytes in the final population of T lymphocytes according to the absence of fluorescence of the n minus 1 chosen parameters or,
   (iiic) T-lymphocytes present in a sample of T-lymphocytes of the initial population not co-incubated with APCs,
(iv) determining intensity of fluorescence of non-proliferating lymphocytes by measuring the fluorescence of the probe used to measure the proliferation either from:
   non-proliferating T-lymphocytes present in the lymphocytes subsets of interest defined at step (ii), from the fluorescent curve defined at step (iiia) or,
   non-proliferating T-lymphocytes present in a lymphocytes subset defined by lymphocytes gated in the final population of T lymphocytes according to the absence of fluorescence of the n minus 1 chosen parameters, from the fluorescent curve defined at step (iiib) or,
   non-proliferating T-lymphocytes presenting in a sample of T-lymphocytes of the initial population not co-incubated with APCs, from the fluorescent curve defined at step (iiic),
   (v) deriving, from the fluorescence curve recorded at step (iii), Gaussian curves centered on successive half intensity values derived from the maximal intensity of fluorescence determined from non-proliferating T-lymphocytes at step (iv), to obtain Aₖ which is the proportion of cells in division k at the time of the measure of the proliferation,
(vi) determining the proportion of T-lymphocytes (PF=precursor frequency) in the initial population that have proliferated in order to give the proportion of T lymphocytes present in the selected subset (step ii) using the formula:

$$PF = ( \begin{matrix}k = n \\ \sum {A}_{k} / {2}^{k} \\ k = 2\end{matrix} ) / ( \begin{matrix}k = n \\ \sum {A}_{k} / {2}^{k} \\ k = 0\end{matrix} )$$

wherein PF is precursor frequency in the initial population, Aₖ is the proportion of cells in division k at the time of the measure of the proliferation, k=0 for initial population of T lymphocytes, and cells having undergone 2 to n divisions having been classified as proliferating T lymphocytes,
(vii) determining the percentage of non-proliferating T-lymphocytes from the percentage of T-lymphocytes that have not proliferated and that are present in the final population of T-lymphocytes and half the percentage of T-lymphocytes that had undergone only one cell division,
(viii) applying the percent of non-proliferating T-lymphocytes to the number of gated T-lymphocytes in the data file to give the absolute number of T-lymphocytes in the corresponding subset before culture that will not proliferate according to the formula,
   number_{non-proliferating cells in the initial population}=[(proportion_{cells that have not proliferated and that are present in the sample at the end of the experiment})+(0.5*proportion_{cells that have divided only once and that are present in the sample at the end of the experiment})]*[number_{gated cells in data file}],
(ix) determining the absolute number of T-lymphocytes in the corresponding subset destined to divide by knowing the number of T-lymphocytes that was not destined to divide and the number of precursor cells of proliferating T-lymphocytes according to the formula,
   number_{proliferating cells in the initial population}=[(PF_{proliferating cells})*(number_{non-proliferating cells in die initial population})]/[1−PF_{proliferating cells}],
(x) reiterating step (i) to step (vii) to each T lymphocytes subsets determined according to the n parameters used for the measure,
(xi) summation of number of cells in the 2ⁿ subsets in order to express the number of T-lymphocytes present in the initial population of T-lymphocytes as a percentage of the total initial population before co-incubation.

The man skilled in the art determines visually, according to the distribution of the cells along the curve of fluorescence recorded at step (iii), the maximal value of fluorescence corresponding to the non-proliferating cell. However for such determination, a minimum number of events (cells displaying the maximal value of fluorescence namely non-proliferating cells) are required. For example, it may be required that at least 50 events, more preferably at least 100, more preferably at least 500, more preferably at least 5000, have to be distributed around the maximal value of fluorescence that allows the determination of the fluorescence of the non-proliferating cells. The tern “around” should be understood as meaning that the fluorescence of the cells considered as being non-proliferating have not to differ from about 70%, from about 60%, from about 50%, from about 25%, more preferably from about 20%, more preferably from about 10%, more preferably from about 5%, more preferably from about 2%, from the value of fluorescence considered as being maximal. In some cases the number of non-proliferating cells in the subset gated in step (iiia) may be not sufficient to determine manually the value of fluorescence of non-proliferating cells. A population of primary T-cells (directly taken from the blood of an animal, for example) that may have been previously in contact with the antigen used to load the antigen presenting cells may contain a great number, at least about 0.001%, at least about 0.01%, at least about 0.02%, at least about 0.1%, at least about 0.2%, at least about 0.5%, at least about 1%, at least about 5%, at least about 10%, at least about 20%, at least about 30%, at least about 40%, at least about 50%, at least about 60%, at least about 70%, at least about 80%, at least about 90% of T-cells able to respond to the contact with the antigen-presenting cells. Those cells may highly proliferate, namely divide at least once, more preferably at least twice, at least 3 times, at least 4 times, at least 5 times, at least 6 times, at least 7 times, at least 8 times, at least 9 times, at least 10 times, at least 15 times, at least 20 times, after contact with the antigen-presenting cells and the number of non-proliferating cells, in the subset gated at step (iiia), will be insufficient to determine the value of fluorescence corresponding to the non-proliferating cells. In such case it may be possible to determine the maximal value of fluorescence in another subset of T-lymphocytes present in the final population but from which it may be known a priori that there are few proliferating cells, for example less than about 50%, less than about 40%, less than about 30%, less than about 20%, less than about 10%, less than about 5%, less than about 2%, less than about 1% in respect with the total number of cells. Such subset of T-cells may be a subset in which the T-cells are negative for the n minus 1 chosen parameters (step iiib). The T-cells negative for the n minus 1 chosen parameter are likely to be the cells that do not respond to the stimulus represented by the antigen-presenting cells and therefore the cells that do not proliferate.

Another example where the number of non-proliferating cells will be insufficient to determine the value of fluorescence corresponding to the non-proliferating cells is when the initial population of T-lymphocytes is constituted of a clone population or a cell line. In such case, virtually all the cells have the ability to proliferate and therefore there will likely be substantially no non-proliferating cells, for example less than about 50%, less than about 40%, less than about 30%, less than about 20%, less than about 10%, less than about 5%, less than about 2%, less than about 1% in respect with the total number of cells. Therefore, in such case, the man skilled in the art may establish the value of fluorescence of non-proliferating cells from a sample non-submitted to the contact with the antigen-presenting cells loaded with an antigen (step iiib).

It should be noted that in step (vi), the generation 1 has been excluded from the calculation in order to reduce the artefact caused by the width of the parental generation or the slow proliferation occurring after long culture periods.

However, in some cases it may be useful to include the first generation of dividing cells in the calculation. It may be useful to include the first generation of dividing cells in cases where the contact between APCs and the initial population of T lymphocytes results in a rapid proliferation.

In such cases the formula to be used at step (v) will be as follows:

$$PF = ( \begin{matrix}k = n \\ \sum {A}_{k} / {2}^{k} \\ k = 1\end{matrix} ) / ( \begin{matrix}k = n \\ \sum {A}_{k} / {2}^{k} \\ k = 0\end{matrix} )$$

wherein PF is precursor frequency in the initial population, Aₖ is the proportion of cells in division k at the time of the assay, k=0 for initial population of T lymphocytes, and cells having undergone 1 to n divisions having been classified as proliferating cells

And in step (ix), the formula to apply will be as follows:
number_{non-proliferating cells in the initial population}=[proportion_{cells that have not proliferated and that are present in the sample at the end of the experiment}]*[number_{gated cells in data file}],

The man skilled in the art will know from its experience when to include or exclude the first generation of dividing cells from the calculation.

In the method described above, steps (i) to step (iv) are made by using any flow cytometry apparatus known from the man skilled in the art and steps (v) to (vii) are easily made in using ModFit software version 3.1 (Verity Software House, Topsham, Me.). The present part of the invention will be more clearly presented in the preferred embodiment section.

The present invention allows also the determination of an index of proliferation (PI). The index of proliferation is indicative of the proliferative potential of the resting cells as they existed at time zero. PI is a measurement of the degree of cells expansion that result from a ratio of total number of cells in final population to the total number of cells before stimulation. The index of proliferation is determined by knowing the number of cells that were present in the initial population and the number of cells that are present in the final population according to the formula:

$$PI = ( \begin{matrix}k = n \\ \sum {A}_{k} \\ k = 0\end{matrix} ) / ( \begin{matrix}k = n \\ \sum {A}_{k} / {2}^{k} \\ k = 0\end{matrix} )$$

wherein Aₖ is the proportion of cells in division k

The T-cell receptor (TcR) for an antigen is a member of the immunoglobulin superfamily. TcRs, recognize peptide fragments presented in the context of MHC molecule class I and II found on the surface of APCs. The structure of the TcR is similar to the structure of an antibody and also varies in one region so that each TcR is unique. Hence a T cell antigen receptor is specific to an antigen/MHC combination. The probe used to detect the presence and the level of T cell antigen receptor on the surface of T lymphocytes may be fluorocldrome labeled MHC-peptide tetramers. The fluorochrome that may be used are for example FITC, PE, PerCP or allophycocyanin. The MHC-peptide tetramers may be MHC class-I peptide tetramers for CD8⁺ T cells or MHC class-II peptide tetramers for CD4⁺ T cells. Tetramers may be generated using now well-established procedures known from the man skilled in the art (Kotzin et al., Proceed Natl Acad Sci USA 2000. 97:291-6; Novak et al., J Clin Invest. 1999 104:R63-7, Ge et al., Proceed Natl Acad Sci USA 2002. 99:13729-34; Mylin et al., J Virol 2000. 75:6922-34). It should be noted that, according to the invention, the T cell antigen receptor whose presence is detected on the surface of T lymphocytes may be or may not be specific to the antigen, or fragment of antigen, loaded into the APCs.

In a particular embodiement of the present invention the T cell antigen receptor on the surface of T lymphocytes is specific for an antigen or of a fragment of antigen loaded on the APCs.

The T cell antigen receptors whose presence is detected on the surface of T lymphocytes according to the present method may be specific for antigen coming from a tumor or an infectious agent or a self-antigen. The followings are non limited examples of tumoral antigen the T cell antigen receptors may be specific for: p53, Melan-A MART-1, MAGE-3, MAGE-2, PSA, PSMA, PAP, HSP70, CEA, Ep-CAM, MUC1, MUC2, HER2/neu peptides or modified peptides derived from this proteins.

The followings are non limited examples of antigen from infectious agent the T cell antigen receptors may be specific for: Flu peptide (M1₅₈₋₆₆ peptide (GILGFVFTL) derived from the M1 protein of the influenza virus), proteins from tetanus toxoid, EBV (Epstein Barr Virus), Cliff (cytomegalovirus), HBV (hepatitis B virus) or HIV peptides or modified peptides derived from these proteins.

An antigen-presenting cell is a cell that recognizes an antigen, processes it, and incorporates the resulting peptides into the major histocompatibility complex (MHC) molecules on the cell surface. The resulting MHC-peptide complexes are then presented to T-lymphocytes. The antigen-presenting cells (APCs) loaded with at least one antigen, or fragment of antigen, may be monocytes or monocyte-derived antigen presenting cells. Those APCs may also be immature, maturing or mature dendritic cells (DC). Those APCs may also be monocytes or macrophages. Those APCs may also be B-lymphocytes or other bone marrow derived-cells.

Monocytes may be obtain from blood sample through any known technique of the art. Monocytes may be isolated from peripheral blood mononuclear cells (PBMCs) or from bone-marrow. Monocytes may be differentiated in immature DCs by incubation in presence of GM-CSF and IL-4 or GM-CSF and IL-13. When differentiated by incubation with GM-CSF and IL-13, the lymphocytes that were present in PBMCs are preferentially left with the monocytes during the differentiation. Monocytes may be differentiated in macrophages by culturing them in the presence of GM-CSF and IFN-γ. Those cells are obtained according to any methods known from the man skilled in the art or methods such as those described in U.S. Pat. No. 5,804,442, WO 94/26875, WO 97/44441 or WO 02/055675 or WO 03/010301.

In the event that lymphocytes are present during the differentiation of monocytes, they are eliminated after the differentiation and before the use of the differentiated-monocytes for the purpose of the invention.

Maturing DC according to the present invention should be understood as DC in which the process of maturation has been triggered but who have not reached the state of full maturation. Immature DC are characterized by presence of surface determinants markers specific to their immature state such CD14 or by absence of others surface determinants markers that in contrary are specific to a mature state such as CD83. Thus maturing DC should be understood as cells presenting intermediary expression of markers from immature to mature state.

Those cells are obtained according to any methods known from the man skilled in the art or methods such as those described in U.S. Pat. No. 5,804,442, WO 94/26875, WO 97/44441 or WO 02/055675 or WO 03/010301. For example mature dendritic cells (DC) may be obtained by culturing immature DC in presence of maturation agents such as bacterial extracts alone or in combination with IFN-γ, or polyriboinosinic-polyribocytidylic acid (polyI:C) and anti-CD40 mAb.

In another embodiment of the present invention the APCs may be loaded with at least one antigen or a fragment of antigen which is an antigen of tumoral or infectious origin. The APCs may be loaded with one given antigen or with a mixture of antigens, or fragment of antigen(s), or with a plasmid containing a gene coding for a protein of interest. This protein being able to be processed in order to be presented at the surface of APCs associated with MHC molecules. The antigens or fragment of antigens or proteins of interest may be of tumoral or infectious origin. It should be noted that the T cell antigen receptor measured on the surface of T lymphocyte, according to the present invention, may be specific or not to an antigen, or fragment of antigen, used to load APCs.

Methods for loading APCs are those which are known from the man skilled in the art. For example, methods may comprise addition of the culture medium of APCs with crude antigens, for instance autologous tumor membrane, killed tumoral cells, bacterial capsides, viral homogenates cleared from nucleic acids, specific peptides against which an immune response is desired, cDNA or genetic material linked to vectors to allow transfection of the APCs with material coding for the relevant peptide or protein to be presented on the APCs membrane and against which an immune response is desired,

According to a particular mode of the invention, the antigen, or fragment of antigen, used to load the APCs may be an antigen originating from tumoral cells or tissues. For example such antigen may be p53, Melan-A MART-1, MAGE-3, MAGE-2,PSA, PSMA, PAP, HSP70, CEA (carcinoma embryonic antigen), Ep-CAM, MUC1, MUC2, or HER-2/neu or peptides derived from these proteinic antigen, all known from the man skilled in the art.

According to another particular mode of the invention, the antigen, or fragment of antigen, used to load the APCs may also be an antigen originating from infectious agents such as bacteria, viruses, fungus or proteinaceous infectious agent. For example such antigen may be Flu peptide (M1₅₈₋₆₆ peptide (GILGFVFTL) derived from the M1 protein of the influenza virus), tetanus toxin, EBV, CMV, HBV or peptides derived from these proteinic antigen.

It should be noted that, according to the invention, the antigen, or fragment of antigen, loaded into the APCs may be or not related to the T cell antigen receptor whose presence is detected on the surface of T lymphocytes.

The co-incubation step between APCs and T lymphocytes should last a time to allow a sufficient number of cells divisions, that is to say a time sufficient to allow at least 1 division, preferably at least 2 divisions, and more preferably 5 divisions. This time may range from 1 to 10 days, and more preferably from 4 to 10 days depending on the T cell response to the antigen being studied.

At the end of the co-incubation period, in order to obtain a detectable level of biological molecules that were produced by T lymphocytes (such as cytokines and/or chemokines and/or enzymes), the T cells may undergo a restimulation period. This restimulation may be a step of adding APCs loaded with an antigen, or a fragment of antigen or a polyclonal activator such as PMA and ionomycin. This restimulation step may intervene approximately 16 hours before the end of co-incubation period.

According to the invention, the proliferation of T lymphocytes is assessed by using a probe loaded into T lymphocytes before or concomitantly to the step of co-incubation.

According to a particular mode of the invention the cell proliferation may be determined using fluorescent probes that are added to T lymphocytes before the step of co-incubation. Those are fluorescent dyes that stain the cytosol or the lipid bilayer of the outer membrane. Those probes are substantially equally distributed between dividing T lymphocytes during cell division of cells derived from the T lymphocytes of initial population. The fluorescent probes that stain the cytosol are for example CFSE (carboxyfluorescein diacetate, succinimyl ester or CFDA-SE). The fluorescent probes that stain the lipid bilayer of the outer membrane are for example PKH67 or PKH26 or Di-O, Di-I.

According to another particular mode of the invention the cell proliferation may also be determined using probes that are added to T lymphocytes concomitantly to the step of co-incubation, and that are detected at the step of the flow cytometry analysis using specific antibodies directed against them. Those antibodies may be labelled with a fluorescent molecules or may be the target of secondary antibodies which are labelled by fluorescent molecules. Such probes are for example the Bromo-d-Uracile (BrdU) known from the man skilled in the art.

A possible use of this new method is to set up a potency assay of a composition of APCs. A potency assay is an assay that determines the specific ability or capacity, as determined by appropriate laboratory tests or adequately controlled clinical data obtained through the administration of the product in the manner intended, to effect a given result. This potency assay comprises the determination of proliferation index of the T lymphocytes and/or the determination of the proportion of T lymphocytes precursors present in the initial population as characteristics of the capacity of APCs to activate those T lymphocytes. The assay measures the fraction of antigen specific T-cell markers positives (e. g. tetramers) cells which are proliferating and differentiating along a defined immune pathway (e.g. Th-1 or Th-2 pathway).

According to the use of the present method as a potency assay, the APCs should be able to induce a proliferation index of the (P⁺, TCR⁺ T lymphocytes of at least at least 2, more preferably of at least 5, more preferably of at least 10, more preferably of at least 15, more preferably of at least 20, more preferably of at least 30, more preferably of at least 50.

In an other particular embodiment of the invention, the APCs should be able to induce a proliferation index of the (P⁺, TCR⁺) T lymphocytes ranging between 2 and 200, more particularly from 15 to 70, more particularly from 20 to 60, more particularly from 30 to 40, more particularly from 20 to 200.

According to the use of the present method as a potency assay, the APCs should be able to induce a proliferation index of the (P⁺, C⁺) T lymphocytes of at least at least 2, more preferably of at least 5, more preferably of at least 10, more preferably of at least 15, more preferably of at least 20, more preferably of at least 30, more preferably of at least 50.

In an other particular embodiment of the invention, the APCs should be able to induce a proliferation index of the (P⁺, C⁺) T lymphocytes ranging between 2 and 200, more particularly from 15 to 70, more particularly from 20 to 60, more particularly from 30 to 40.

According to another particular mode of the invention, the APCs should be able to induce a proliferation index of the (P⁺, TCR⁺, C⁺) T lymphocytes of at least 2, more preferably of at least 5, more preferably of at least 10, more preferably of at least 15, more preferably of at least 20, more preferably of at least 30, more preferably of at least 50.

In an other particular mode of the invention, the APCs should be able to induce a proliferation index of the (P⁺, TCR⁺, C⁺) T lymphocytes ranging between 2 and 200, more particularly from 15 to 70, more particularly from 20 to 60, more particularly from 30 to 40, more particularly from 20 to 200.

According to another mode of the invention, the APCs should be able to induce a proliferation index of the (P⁺, TCR⁺, A⁺) T lymphocytes of at least 2, more preferably of at least 5, more preferably of at least 10, more preferably of at least 15, more preferably of at least 20, more preferably of at least 30, more preferably of at least 50.

According to another mode of the invention, the APCs should be able to induce a proliferation index of the (P⁺, TCR⁺, A⁺) T lymphocytes ranging between 2 and 200, more particularly from 15 to 70, more particularly from 20 to 60, more particularly from 30 to 40.

According to another mode of the invention, the APCs should be able to induce a proliferation index of the (P⁺, C⁺, A⁺) T lymphocytes of at least 2, more preferably of at least 5, more preferably of at least 10, more preferably of at least 15, more preferably of at least 20, more preferably of at least 30, more preferably of at least 50.

According to another mode of the invention, the APCs should be able to induce a proliferation index of the (P⁺, C⁺, A⁺) T lymphocytes ranging between 2 and 200, more particularly from 15 to 70, more particularly from 20 to 60, more particularly from 30 to 40, more particularly from 20 to 200.

According to another mode of the invention, the APCs should be able to induce a proliferation index of the (P⁺, TCR⁺, C⁺, A⁺) T lymphocytes of at least 2, more preferably of at least 5, more preferably of at least 10, more preferably of at least 15, more preferably of at least 20, more preferably of at least 30, more preferably of at least 50.

According to another mode of the invention, the APCs should be able to induce a proliferation index of the (P⁺, TCR⁺, C⁺, A⁺) T lymphocytes ranging between 2 and 200, more particularly from 15 to 70, more particularly from 20 to 60, more particularly from 30 to 40.

The potency assay defined according to the present invention may serve as measures of quality control.

A possible use of this new method is to set up a method to characterize effector molecules secreted by, and/or on the surface of APCs, which are responsible for inducing proliferation and/or differentiation and for polarization of T cells from an initial population of T lymphocytes (e.g. Th1 or Th2 pathway). By defining the final population of T lymphocytes the contribution of effector molecules secreted and/or on the surface of APCs, APCs potency is assessed.

Molecules secreted by APC may be for example cytokines as IL-2, IL-10, IL-12, IL-15, IL-18, IL-23, TNF-α, TGF-β. Molecules on surface of APCs may be for example B7, OX-40 ligand, CD40, ICAM-1, 4-1BBL, DC-SIGN.

The evaluation of the effect of molecules secreted by APCs on T cells proliferation could be done for example by addition of specific antibodies, agonist or antagonist ligands, that may bind to those molecules or to receptors for those molecules present on surface of T-cells during the co-incubation between APCs and T-cells. Those specific antibodies may have a blocking effect when they bind to those molecules or to receptors of those molecules, notably by hindering normal interactions between those molecules and their corresponding receptors. Those specific antibodies may also have an activating effect when they bind to receptors of a given molecule by acting in place of the said molecule.

Some molecules present on surface of APCs may intervene to direct the immune response observed in a final population of T lymphocytes resulting from the incubation of those APCs with an initial population of T lymphocytes. Effect of those molecules may be assessed by blocking them with specific antibodies in order to prevent their interactions with other molecules present on the surface of T-cells (cell-cell interactions) or with activating molecules secreted by APCs (autocrine activation) themselves or by T lymphocytes (paracrine activation). In an other of way of testing, those molecules may be activated by agonistic antibodies.

An altered immune response is a response obtained by incubating APCs with an initial population of T cells in presence of any components that is different from the response obtained in the same conditions but without those said components. Observation of an altered immune response in final population of T lymphocytes resulting from co-incubation of APCs with an initial population of T lymphocytes in the presence of blocking antibodies specific for a molecule secreted by APCs may reflect the importance of this said molecule for the potency of APCs to direct T cells of an initial population of T lymphocytes toward a particular immune response in the final population of T lymphocytes.

An other possible use of this new method is to set up an assay in order to evaluate the effect of one or more surface determinants markers present on T-cells on a T-cell response resulting from the co-incubation with a composition of APCs. According to this particular use the surface determinant markers to be blocked may be receptors for cytokines (as described above) or receptors for chemokines or receptors that mediate intracellular signal in response to cell-cell interaction. Those surface determinant markers are blocked by using antibodies or antagonists. Examples of such surface determinants markers that may be the object of the present application are CD4, CD8, CD28, CTLA-4, B7, LFA-10, OX40-ligand or MHC-II.

A possible use of this new method is to set up a batch release assay of a composition of APCs. This batch release assay comprises the determination of the capacity of APCs to induce the production of different cytokines into a population of T lymphocytes. The APCs are characterized by determining the percentages of T lymphocytes which secrete the cytokines that make a population of T lymphocytes preferably acquire a cytotoxic effector function over an helper effector function. Some cytokines are known to be preferably characteristic of a Th1 rather than a Th2 or a Th3 response such as IFN-γ or IL-2 whereas others cytokines are known to preferably induce a Th2 response such as IL-4 or IL-10. Such cytokines are known to induce an immunostimulatory response over an immunosuppressive response. The determined percentage of T lymphocytes secreting such cytokines may be used as an index of the capacity of APCs to induce a particular immune response. The same assay combining some of the others parameters (e.g. proliferation, cytokine production, detection of a specific TcR, detection of one or more surface determinant markers others than TcR, detection of chemokines, detection of enzymes) allows to quantify a) the capacity of APCs to prime naive T cells, b) the antigen specific proliferation and c) the quality of the response (Th-1 or Th-2) obtained.

A possible use of this new method is to determine an inclusion criteria for a patient. An inclusion criteria is a criteria that establishes whether a person is eligible to participate in a clinical trial or to be subjected to a particular treatment. In particular, one advantage of such use is to establish that the patient is not anergic, namely, unable to respond to an antigen. In such use, the composition of APCs presenting target specific antigens and originating from the patient should have the ability to induce a proliferation of one or more subsets of T lymphocytes of interest resulting in a PI at least greater than 2, more preferably of at least 5, more preferably of at least 10, more preferably of at least 15, more preferably of at least 20, more preferably of at least 30, more preferably of at least 50.

In a more particularly use, the composition of APCs originating from the patient should have the ability to induce a proliferation of one or more subsets of T lymphocytes of interest resulting in a PI ranging between 2 and 200, more particularly, from 15 to 70, more preferably from 20 to 60, more particularly from 30 to 40, more particularly from 20 to 200.

An other possible use of this new method is to set up an antigen selecting assay. In this particular use of the invention the antigen to be tested is loaded on APCs and the T lymphocyte response triggered by the co-incubation with those APCs is compared to the T lymphocyte response induced by a composition of APCs loaded with a reference antigen. In this particular use, the T cell antigen receptor detected on the surface of the T lymphocytes should be specific for the antigen used to load the [symbol] Examples or antigens against which T-cells response may be assayed are p53, Melan-A/MART-1, MAGE-3, PSA, PSMA, PAP, HSP70, HSP70 derived peptides, CEA (carcinoma embryonic antigen), Ep-CAM, MUC1, MUC2, or HER2/neu, all known from the man skilled in the art.

The reference antigen used according to this particular use of the invention may be for example tetanus toxin, Melan-A, Flu peptide, PSA (when APCs and T cells come from an healthy woman), HIV (when APCs and T cells come from an HIV-sero-negative person).

In a more particular embodiment of the invention, the antigen selecting assay is specific to a patient and is used to design a patient's specific vaccine.

An other possible use of this new method is to set up a diagnostic assay in order to detect in a given patient the presence of pathogenic T lymphocytes. Pathogenic T lymphocytes being, as defined above, able to induce autoimmune disease. In a such test, APCs isolated from the patient would not be loaded with antigen in vitro. After isolation and possibly maturation, APCs are incubated in presence of T-cells originating from the same patient. A T-cell response observed will be indicative of the presence of pathogenic T cells.

Another possible use of the method is to define a standard control T-cell response of T-lymphocytes. This standard T-cell response of T-lymphocytes may be further used to compare some factors that may play a role on the presentation of the antigens by the antigen presenting-cells, such as, but not limited to, processes for antigen loading, qualification of antigens batches, stability of the presentation of the antigen by the antigen-presenting cells. This standard control T-cell response of T-lymphocytes comprises
the co-incubation of an initial population of T-lymphocytes with different compositions of APCs presenting different concentrations of an antigen or of an antigen fragment of interest or of a reference antigen or of a fragment of reference antigen and,
the determination of the variation of the degree of proliferation of said T-lymphocytes measured for each composition of APCs according the quantity of said antigen or said fragment of antigen of interest or said reference antigen or said fragment of reference antigenresponse wherein a correlation between a degree of proliferation of T lymphocytes and a quantity of antigen, or fragment of antigen, presented in a context of MHC is carried out by contacting an initial population of T lymphocytes with a composition of APCs presenting an increasing or a decreasing concentration of the said antigen, or fragment of antigen.

According to the present invention, the term “standard” should be understood as meaning a value, or a concept, that has been established to serve as a model or rule in the measurement of a quantity or in the establishment of a practice or procedure.

The standard control T-cell response of T-lymphocytes may comprise, in addition to the above-defined parameters that may be measured to define the final population of T lymphocytes, a determination of a correlation between a degree of proliferation of T lymphocytes and a quantity of antigen, or fragment of antigen, presented in a context of MHC.

Therefore another possible use of this new method is to define-a standard control T-cell response of T-lymphocytes wherein a correlation between a degree of proliferation of T lymphocytes and a quantity of antigen, or fragment of antigen, presented in a context of MHC, is carried out by contacting an initial population of T lymphocytes with a composition of APCs presenting an increasing or a decreasing concentration of the said antigen, or fragment of antigen.

The correlation may be expressed as the proliferation index (P) and/or Precursor frequency (PF) in function of the concentration of antigen incubated with the APC. According to the quantity of antigen, or fragment of antigen, presented by the antigen presenting-cells in the context of major histocompatibility complex (MHC), the degree of proliferation or PI and or the PF will be more or less considerable. Therefore, the increase (or decrease) of the quantity of antigen presented by the APCs will result in an increase (or decrease) of the degree of proliferation or PI and or PF. In order to establish the reference condition in relation with a given amount of peptide loaded exogenously on the APC, a large range of peptide concentration is incubated with the APC (up to 100 mM peptide) which has been widely used and are well known by the skilled in the art (Hosken et al., J Immunol. 1989. 142:1079-83). Antigen concentration can be plotted against PI and or PF. Reference process will be selected on the linear portion of the plot before a plateau is reached.

The quantity of antigen presented by the APCs may also be measured by flow cytometry techniques when an MHC/peptide complex-specific antibody is available (Cohen et al., J Mol Recognit. 2003. 16:324-32). The standard control response may be selected within the curve as PI50 or PF50 (condition of antigen presentation able to reach 50% of maximal PI or PF obtained with the reference process).

The standard control T-cell response of T-lymphocytes may be carried out with a defined pre-processed peptide. The term “pre-processed peptide” means that peptide resembles to a peptide that would have been processed from a full-length protein by an antigen presenting-cell. Such pre-processed peptide may synthesized by techniques known by the an skilled in the art. The peptides can be synthesized in solution or on a solid support in accordance with conventional techniques. Various automatic synthesizers are commercially available and can be used in accordance with known protocols (See, for example, Stewart & Young, SOLID PHASE PEPTIDE SYNTHESIS, 2D. ED., Pierce Chemical Co., 1984). Alternatively, recombinant DNA technology can be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989).

One advantage of using pre-processed peptides to establish the standard control response is that it allows to work in condition of saturation. Namely, the incubation of APCs with pre-processed peptides, which bind directly to the MHC, results mainly on the binding of the peptides to the MHC available at the surface of APCs. Whereas, the use of an antigen that has to be processed before being presented by APCs may result, because of the processing, in steadily increase of quantity of complex MHC-peptide at the surface of APCs until all the loaded proteins are processed. When the standard control response of a pre-processed peptide, namely, the response of a population of T lymphocytes and the correlation between a degree of proliferation of T lymphocytes and a quantity of a pre-processed peptide presented in a context of MHC, is well-defined, this pre-processed peptide may be considered as an antigen of reference.

The standard control T-cell response of T-lymphocytes may be used to evaluate the efficiency of a process to load an antigen, or a fragment of antigen, into APCs. In such use, the efficiency of the process to be tested is evaluated by comparing:
a first response being a T-cell response of a final population of T-lymphocytes response induced resulting from the co-incubation of an initial population of T-lymphocytes by with a composition of APCs loaded with an antigen or a fragment of antigen, according to the process to be tested with and,
a second response being a standard control T-cell response of T-lymphocytes resulting from the co-incubation of an initial population of T-lymphocytes with different compositions of APCs loaded with different concentrations of said antigen or said fragment of antigen, or of a reference antigen, or of a fragment of reference antigen according to the process of reference,
   deducing from said comparison between said first and said second responses the difference of efficiency between the process to be tested and the process of referencea standard control response obtained with APCs loaded according to a reference process with the said antigen, or the said fragment of antigen, or with an antigen, or a fragment of antigen, of reference.

The method of reference to load APCs with antigens may be chosen among the group consisting of: fusion, electroporation, incubation, loading with liposomes, loading with virosomes, loading with exosomes (Wolfers et al., Nat Med., 2001, 7:297-303, Bungener et al., Biosci Rep., 2002, 22: 323-38), genetic engineering of antigen-presenting cells (Bubenik et al., Int J Oncol, 2001, 18: 475-8). The incubation method consists of incubating APCs in the presence of antigens. According to the nature of the antigen, the incubation may result in engulfing, processing and then presentation of the antigen in the context of MHC. An antigen is processed by the antigen processing machinery of the APC, where exogenous proteins are degraded within the endo-lysozomal compartment and are thereby loaded onto MHC class II molecules while proteins present in the cytoplasm are degraded mainly by the proteosome, transported into the endoplasmatic reticulum (ER) and thereby loaded on MHC class I molecules (Ramachandra et al., Cell Microbiol. 1999. 1:205-14; Yewdell, Mol Immunol. 2002. 39:139-46). The engulfing step may resort on micropinocytosis, macropinocytosis, phagocytosis or receptor-dependent internalization. The incubation may also result in a direct binding of the antigen to the MHC. The antigens may be in the form of cell lysates, apoptotic bodies, necrotic bodies, proteins, peptides, mRNA or DNA (Fields et al., Proc Natl Acad. Sci USA. 1998. 95: 9482-7; Ashley et al., J Exp Med. 1997. 186: 117-1182; Nestle et al., Nat Med. 1998. 4:328-32; WO 99/58645). Cell lysates, apoptotic bodies, necrotic bodies or proteins are preferably engulfed by APCs during the step of incubation. The peptides, especially when they are pre-processed peptides, tend to bind directly to the MHC. After being engulfed, the mRNA, in the cytosol, is translated into protein, which is afterwards processed and presented by the APCs in a MHC context. After being engulfed, the DNA is directed toward the nucleus, where it is transduced in mRNA. The latter is transported towards the cytosol where it is translated into proteins which is afterwards processed and presented by the APCs in a MHC context. The fusion method consist in fusing antigen-presenting cells with tumor cells by means of a chemical technique, such as PEG, or an electric process, such as electrofusion (Kugler et al., Nat Med., 2000, 6:332-6; Gottfried et al., Cancer Immun., 2002, 2:15). The process of electroporation consists of applying an electrical field to cells in order to create membrane pores allowing the entry of the diverse substances to be loaded into cells (Ponsaerts et al., Leukemia, 2002, 16:1324-1330). All these methods are well known by the person skilled in the art.

The standard control T-cell response of T-lymphocytes may also be used to set up a quality assay for antigen batch. According to this use the quality of the antigen batch to be assayed is evaluated by comparing a T lymphocyte response induced by APCs loaded with the antigen batch to be tested with a standard control T-cell response of T-lymphocytes obtained with APCs loaded with a reference antigen batch or an antigen, or a fragment of antigen, of reference. The antigen from the reference antigen batch has to be of the same type and nature than the antigen from the antigen batch to be tested. For instance, an antigen batch of, for example, tetanus toxoid or Melan-A or Flu peptide or PSA or HIV or a mixture of antigens or a cell lysate that had been once qualified, namely that fulfilled some defined criteria for quality, may be used thereafter to qualify a new antigen batch. This new antigen batch will be qualified if the T cell response, obtained with APCs loaded with it, fulfills the criteria defined with the standard control response obtained with APCs loaded with the reference antigen batch or a fragment of antigen, of reference.

It is known by the skilled person in the art that depending on the process implemented to prepare antigen, the ability of APCs to present antigen may be affected (Strome et al., Cancer Res, 2002, 62:1884-9). Therefore, another possible use of the new method according to the invention is to evaluate the impact of a method of antigen preparation on the ability of antigen-presenting cell to present antigen to T lymphocyte. According to this use the method of preparation of antigen is evaluated by comparing:
a first response being a T-cell response of a final population of T-lymphocytes resulting from the co-incubation of an initial population of T-lymphocytes with a composition of APCs a T lymphocyte response induced by APCs loaded with an antigen or a fragment of antigen, prepared according to the method to be tested with a T lymphocyte response,
a second response being a standard control T-cell response of T-lymphocytes resulting from the co-incubation of an initial population of T-lymphocytes with different compositions of APCs loaded with different concentrations of said antigen or said fragment of antigen, or of a reference antigen, or of a fragment of reference antigen, and a standard response obtained with APCs loaded with the said antigen, or an antigen, or a fragment of antigen prepared according to a method of reference or an antigen, or a fragment of antigen, of reference,
   deducing from said first and said second responses the impact of said method of antigen preparation to be tested on the ability of an antigen-presenting cell to present antigen to T lymphocyte.

The standard control T-cell response of T-lymphocytes may also be used to evaluate stability of a presentation of an antigen (or fragment of antigen) by APCs wherein the said stability is evaluated by comparing:
a first response being a T-cell response of a final population of T-lymphocytes resulting from the co-incubation of an initial population of T-lymphocytes with different compositions of APCs a T lymphocyte response induced by APCs loaded with the said antigen (or fragment of antigen) said compositions of APCs being previously incubated in a medium not initially containing said antigen for different period of time after increasing period of time of incubation of the APCs loaded with the said antigen (or fragment of antigen) in a medium not initially containing the said antigen (or fragment of antigen) to and,
a second response being a standard control T-cell response of T-lymphocytes resulting from the co-incubation of an initial population of T-lymphocytes with composition of APCs loaded with an antigen or a fragment of antigen, or a reference antigen, or a fragment of reference antigen a standard control response obtained with APCs loaded with the said antigen (or the said fragment of antigen) or an antigen (or a fragment of antigen) of reference, said compositions of APCs being not previously incubated in a medium not initially containing said antigen or said fragment of antigen, or a reference antigen, or a fragment of reference antigen,
   deducing from the first and the second responses the stability of a presentation of said antigen (or fragment of antigen) by APCs without further period of time of incubation in a medium not initially containing the said antigen (or the said fragment of antigen) or an antigen (or a fragment of antigen) of reference.

The standard control T-cell response of T-lymphocytes may also be used with, as initial population of T lymphocytes, a clonal population or a cell line of T lymphocytes that is specific to the antigen, or fragment of antigen, presented in the context of MHC.

According to the present invention, the term “clonal population” should be understood as a group of genetically identical cells derived from a single cell. The T-cell receptor (TcR) displays at the surface of each T cell from a clonal population is identical and recognizes specifically a precise portion of a given antigen.

The clonal population may be derived from a general population of T lymphocytes taken from an animal or a human, which may be or not immunized against the given antigen. Owing to the broad versatility of the TcR of the general population of T lymphocytes, it may exist a TcR specific of a given antigen, even if the population has never encountered the said antigen.

The T cells taken from an animal or human comprise a mixture of cells with different specificities against different antigens. The cells are placed in culture with an antigen or antigen-presenting cells in conditions allowing the proliferation of the antigen-specific T cells (for example, culture medium comprising IL-2). The cells that do not recognized the antigen do not proliferate. The proliferating T-cells constitute a T-cell line. According to the present invention, the terms “T-cell line” should be understood as a population of T cells specific for a given antigen, but comprising T cells displaying TcR recognizing different part of the said antigen. A T cell line could be obtained from a general population population of T lymphocytes by sorting antigen-specific T cells by tetramers/multimers.

A clonal T-cell population may be derived from a T-cell line by using the technique of limiting dilution culture. According to this technique the T-cells from the T-cell line are seeded in wells of culture plate at a concentration allowing the likely distribution of only one cell by wells. The antigen, or antigen-presenting cells are added to the wells, allowing the proliferation of the clone.

This technique may be applied directly to the general population allowing directly the obtaining of a T-cell clone.

The standard response may also be used with, as initial population of T lymphocytes, an initial naive population of T lymphocytes, said initial naive population of T lymphocytes being substantially the same for obtaining a standard control response and a response to be compared to the said standard control response.

According to the present invention, the terms “naive population” should be understood as a population of T cells derived from peripheral blood or from bone marrow cells without further selection. The terms “naïve population” doesn't exclude the possibility that the T-lymphocytes could have been in contact with the antigen used to load the antigen-presenting cells before taking the T-cells from the blood.

### BRIEF DESCRIPTION OF THE TABLES AND THE FIGURES

FIG. 1

The frequency of flu-specific CD8⁺ T cells in peripheral blood of influenza-vaccinated donors determined by tetramer staining IFN-γ ELISPOT or the PKH67 dye dilution assay. For the ELISPOT method (stripped histogram), the mean of triplicate flu-stimulated wells (subtracting the value for stimulation with unloaded DC) is represented for each donor. For tetramer staining (black histogram), data represent the percentage of tetramer positive cells among TO-PRO-3⁻ CD8⁺ cells, subtracting the value for the HIV gag tetramer control. For the dye dilution assay (crossed histogram), the precursor frequency of proliferating cells was calculated with ModFit software in cultures stimulated for 6 days with flu-peptide-loaded DC (subtracting the value in control wells). Error bars represent the standard deviation amongst triplicate samples for ELISPOT and tetramer binding and duplicate samples for the dye dilution assay.

FIGS. 2A, 2B

Expansion of flu-specific CD8⁺ T cells visualized by tetramer staining and the PKH dye dilution assay at day 6 of culture. Cells from each donor were labeled with PKH67 fluorescent dye and stimulated with control (unloaded DC, FIGS. 2A and 2B, left panels) or flu-peptide-loaded DC (FIGS. 2A and 2B, right panels). In all plots, the cells were gated on the live lymphocytes. Cells were analyzed on day 6 for tetramer binding (FIG. 2A) and the PKH67 fluorescence profiles (FIG. 2B) of the CD8⁺ cells. In FIG. 2A, the percentages indicated are the TET⁺CD8⁺ cells among live lymphocytes. In FIG. 2B, the proliferating cells are seen as the sub-population with low PKH67 intensity (arrows).

FIGS. 3A, 3B, 3C

Multiparameter flow cytometric analysis of CD8⁺ T cells cultured with flu-loaded or unloaded DC. Dot plots represent CD8⁺ T cells from donor 2 analyzed for PKH67 dye dilution verses tetramer staining (FIG. 3A), PKH67 verses IFN-γ production (FIG. 3B), and IFN-γ production versus tetramer stainin (FIG. 3C). PKH67-labeled cells were cultured with control (FIGS. 3A, 3B, 3C, left panels) or flu-peptide-loaded DC (FIGS. 3A, 3B, 3C, right panels). On day 5, cells were re-stimulated with control or peptide-loaded DC in the presence of brefeldin A. On day 6, cells were stained with tetramers, anti-CD8 antibody, and antibody against intracellular IFN-γ. The plots are gated on CD8⁺ T cells.

FIGS. 4A, 4B, 4C, 4D, 4E, A, 4F

Multiparameter flow cytometric analysis of proliferation and IFN-γ production among tetramer-negative (left column) and tetramer-positive (right column) CD8⁺ cells following culture for 6 days. PKH-labeled cells were stimulated at day 0 with unloaded DC (FIGS. 4A and B), or with flu-peptide-pulsed DC (FIG. 4C-F). At day 5 of culture, some cell suspensions were re-stimulated with peptide-pulsed DC (FIGS. 4E and F). All cultures were analyzed after an additional overnight incubation. Results in this figure are from donor 2.

FIGS. 5A, 5B, 5C

Proliferation patterns of tumor and virus specific CD8 populations in cancer patients. For each patient, PKH67-labeled CD8 cells were cultured in parallel with MEL1 or FLU1 peptide-loaded dendritic cells. PKH67 fluorescence profiles of CD8 cells were analyzed at day 7. Tetramer-PE vs. PKH67 staining gated on CD8 T cells from patient P05 are shown (FIG. 5A, MEL1: left panels; FLU1: right panels). The mean divisions accomplished by precursors (FIG. 5B) and the frequencies at DO of proliferating precursors among epitope-specific T cells (FIG. 5C) are shown for the two patients tested (MEL1: black histogram, FLU1: white histogram).

FIGS. 6A, 6B

Proliferation capacities of epitope-specific CD8 T cells upon stimulation. PKH67-labeled CD8 T cells were stimulated with loaded (FIG. 6A, upper row) or unloaded (FIG. 6A, lower row) dendritic cells. After 7 days, cells were stained with relevant tetramers and analyzed by flow cytometry. Tetramer-PE vs PKH67 stainings gated on CD8 T cells are shown (FIG. 6A, EBV1: left panels; CMV1: central panel; EBV2: right panels). PKH67 profiles of each tetramer positive population were modeled, to evaluate the number of tetramer positive cells in each generation, called Tₖ (FIG. 6B, EBV1: left panels; CMV1: central panel; EBV2: right panels).

FIGS. 7A, 7B, 7C, 7D

Proliferation patterns of distinct CD8 populations. For each donor, PKH67-labeled CD8 cells were cultured in parallel with EBV1, EBV2 or CMV1 peptide-loaded dendritic cells. PKH67 fluorescence profiles of CD8 cells were analyzed at day 7. The distribution of tetramer positive cells (EBV1: closed triangle; EBV2: closed square) in the different generations at day 7 (FIG. 7A) and the corresponding precursor distribution (FIG. 7B) are shown for one representative experiment. The mean divisions accomplished by precursors (FIG. 7C) and the frequencies at DO of proliferating precursors among epitope-specific T cells (FIG. 7D) are shown for the different donors tested (EBV1: gray histogram; EBV2 white histogram; CMV1: black histogram).

FIG. 8

Kinetics of cytokine secretion by maturing DC. DC were treated with polyI:C/anti-CD40 mAb (dark-gray histogram), bacterial extract (white histogram), bacterial extract+IFN-γ (black histogram) or mock-treated (gray) (for 3, 6, 20, or 40 h. Culture supernatants were collected (0-3 h, 0-6 h, 0-20 h, and 6-40 h in the figure), and, after a gentle wash, DC were farther cultured in the absence of maturation agents until 40 h (3-40 h, 6-40 h, 20-40 h in the figure). Cytokine concentrations were measured by ELISA in supernatant. IL-2, TGF-β, IL-4, and IL-7 were undetectable. Data are representative of 3 experiments.

FIGS. 9A, 9B

Short DC treatment with bacterial extract and IFN-γ allows for induction of high frequencies of Melan-A-specific CTL. DC were exposed to bacterial extract and IFN-γ for 3, 6, or 20 h, pulsed with Melan-A peptide, washed, and used to stimulate autologous purified CD8⁺ T cells. Alternatively, DC were pulsed with peptide, then maturation agents added to DC and T cells cocultures (maturation “during priming”). Eight T cell microwells were stimulated for each DC condition. After 2 stimulations, T cells were tested by IFN-γ ELISPOT (FIG. 9A, closed rhombus) or ⁵¹Cr-release assay (FIG. 9B, E/T=40/1) against T2 cells pulsed with Melan-A (FIG. 9B, closed circle) or control PSA1 peptide (FIG. 9B, open circle). Shown are average and SD from the 8 microcultures independently tested. In the ELISPOT, background with T2-control peptide was subtracted from specific SFC. Data are representative of 3 experiments.

FIGS. 10A, 10B, 10C

Presence of IFN-γ during DC maturation or addition of exogenous cytokines during T cell stimulation are required for optimal priming. DC were exposed to polyI:C/anti-CD40 mAb, bacterial extract, or bacterial extract+IFN-γ for 6 h (FIG. 10A) or 20 h (FIGS. 10B, 10C), pulsed with Melan-A peptide, washed, and used to stimulate autologous purified CD8⁺ T cells. Alternatively, maturation agents were added to peptide-pulsed DC and T cells cocultures (FIGS. 10A, 10B, 10C, “maturation during priming”). Eight T cell microwells were stimulated per each DC condition. After 3 stimulations, CD8⁺ T cells were tested for IFN-γ secretion in ELISPOT against T2 cells pulsed with Melan-A or control PSA1 peptide. Shown are average and SD from the 8 microcultures independently tested (FIGS. 10A, 10B, closed rhombus). Background with T2-control peptide was subtracted from specific SFC. For DC matured for 6 h in the presence or absence of IFN-γ, difference is statistically significant (p<0.05, Student t test). FIG. 10C: IL-12 and IL-6 were added during the first stimulation, IL-2 and IL-7 during the following one. CD8⁺ T cells were then tested for specific cytotoxicity in ⁵¹Cr-release assay against T2 cells pulsed with Melan-A (closed circle) or control PSA1 peptide (open circle) (E/T=50/1). Data in FIG. 10B and FIG. 10C were generated with cells from the same donor. Data are representative of 3 experiments.

FIG. 11

DC activated for 6 h with different maturation agents induce Melan-A-specific CTL with similar avidity. DC were exposed to polyI:C/anti-CD40 mAb (black cross), bacterial extract (closed circle), bacterial extract+IFN-γ (closed triangle) or to no maturation agent (open rhombus) for 6 h, pulsed with Melan-A peptide, and used to stimulate autologous purified CD8⁺ T cells. After 2 stimulations, specific cytotoxicity was tested against T2 cells (E/T=40/1) in presence of different concentrations of Melan-A or control PSA1 peptide. For easier comparison, data are shown as percent maximal specific lysis for each condition (calculated as: [(specific lysis-minimal specific lysis)/(maximlial specific lysis-minimal specific lysis)]×100), with maximal specific lysis being: 6% for non-matured DC, 8.5% for polyI:C/anti-CD40, 37% for bacterial extract, 47% for bacterial extract+IFN-γ. Background lysis of T2-control peptide was <2%. Data are representative of 2 experiments.

FIG. 12

Melan-A-specific CD8⁺ T cells generated by DC activated with different maturation agents acquire a CCR7⁻/CD45RA⁻ effector memory phenotype. DC were exposed to mock stimulation (2^{nd} left hand plot), polyI:C/anti-CD40 mAb (3^{rd} left hand plot), bacterial extract (4^{th} left hand plot), or bacterial extract+IFN-γ (5^{th} left hand plot), for 6 h, pulsed with Melan-A peptide, washed, and used to stimulate autologous purified CD8⁺ T cells (HD122, same donor as in FIG. 11). Before stimulation (first left hand plot), or after 2 stimulations with DC (plots 2 to 5), CD8⁺ T cells were stained with A2-/Melan-A tetramers, anti-CD8, anti-CCR7, and anti-CD45RA mAb. Data are gated on tetramer⁺, CD8⁺ lymphocytes. Among CD8⁺ cells, Melan-A-specific cells were 0.09% before stimulation, and: 0.35, 1.45, 16, 51% for, respectively, non-matured, polyI:C/anti-CD40, bacterial extract, bacterial extract+IFN-γ-matured DC. For sake of clarity, 100% of tetramer⁺/CD8⁺ events are shown in plots 1 to 3, 20% in plots 4 and 5. Data are representative of 3 experiments.

FIGS. 13A, 13B, 13C

Melan-A pulsed “maturing” DC induce IL-12-dependent proliferation of Melan-A-specific but not influenza or EBV-specific CD8⁺ cells. Purified CD8⁺ cells were labeled with PKH67, then stimulated with Melan-A or PSA1-pulsed DC that were treated with bacterial extract and IFN-γ. After 8 days of culture, T cells were stained as indicated in Example 3. FIG. 13A Maturation for 3 (2^{nd} and 3^{rd} left plots), 6 (4^{th} left plot), or 20 h (5^{th} left plot), or during priming (6^{th} left plot) or no maturation (1^{st} left plot). FIG. 13B Maturation during priming. FIG. 13C Maturation for 6 h, T cell stimulation in the presence of isotype controls (FIG. 13C, left plot) or anti-IL-12 and IL-12Rβ1 blocking mAbs (FIG. 13C, right plot). Data are gated on: (FIG. 13A) viable CD3⁺ (CD4⁺/CD8⁻ cells were <0.02% among CD3⁺ cells) or (FIGS. 13B, 13C) CD8⁺ lymphocytes. Control with PSA1-pulsed DC is shown only for 3 h-matured DC (FIG. 13A, 3^{rd} left plot) for sake of clarity but was routinely performed for each condition of stimulation and proliferation of Melan-A-specific CD8⁺ T cells was never detected. Experiments with cells from 3 different healthy donors are shown in FIGS. 13A, 13B and FIG. 13C.

FIGS. 14A, 14B

Stimulation of Melan-A specific T cell clone with DC loaded Melan-A peptide. 3×10⁴ HLA-A2 positive human DC matured 6 hours with FMKp and IFNγ were loaded with Melan-A_{26-35 (27L)} peptide (10 μg/ml) (FIGS. 14A and 14B upper row panel) or irrelevant PSA1 peptide (10 μg/ml) (FIGS. 14A and 14B lower row panel) and co-incubated with 3×10³ Melan-A specific T cell clone labeled with PKH-67, in the presence of IL-2 and supernatant of MLA cell line. After 6 days, T-cells were labeled with anti-CD8 antibody and tetramer specific for Melan-A_{26-35 (27L)} peptide and sorted by flow cytometry (FIG. 14A: Melan-A_{26-35 (27L)} upper panel; PSA1 lower panel). FIG. 14B Distribution of the fluorescence of the probe used to measure the proliferation of T-cells (PKH67) according to the cells number (FIG. 14B: Melan-A_{26-35 (27L)} upper panel; PSA1 lower panel). The precursor frequencies and proliferation indices were calculated with modFit software. FIG. 14B: Melan-A _{26-35 (27L)} upper panel Proliferation index: 3.54; Precursor frequency: 86.5%. FIG. 14B: PSA1 lower panel Proliferation index: 1.12; Precursor frequency: 3.4%.

Table I

The proportion of CD8⁺ cells in each of the eight sub-populations on day 6 and also the precursor frequencies calculated for day 0. Data are from cultures of cells from donor 2 stimulated with flu-peptide-loaded DC. Each column of figures adds up to 100%, accounting for all the TET⁺ or TET⁻ cells on either day 0 or day 6.

Table II

Precursor frequencies of CD8⁺ cells. The percentages for flu-stimulated or control cells add up to 100%, thus describing all the cells in the resting (day 0) culture with respect to their ability to respond to influenza (or control) stimulation by cytokine synthesis and/or proliferation. The data obtained for donors 1, 2, and 3 are presented with standard deviations.

Table III

Precursors frequencies (PF, percentage) and proliferation indexes (PI) of Melan-A-specific CD8⁺ T cells proliferating after stimulation with non-matured DC or DC treated with bacterial extract and IFN-γ for 3, 6, 20 h or during priming.

Table IV

Precursors frequencies (PF, percentage) and proliferation indexes (PI) of Melan-A-specific CD8⁺ T cells after stimulation with non-matured DC or DC treated for 6 h with polyI:C/anti-CD40 or bacterial extract in the presence or absence of IFN-γ.

### DETAILED DESCRIPTION OF THE PREFERRED EMBODIEMENTS

### EXAMPLE 1

Here we illustrate the method according to the invention, based on multiparameter flow cytometry, to visualize simultaneously proliferation and cytokine production by T cells having different capacities to bind MHC/peptide tetramers; this method also allows calculations of the original frequency of these sub-populations of cells ex vivo.

Antigens encountered by T cells affect their proliferation potential and drive acquisition of effector functions including cytokine synthesis and cytolytic activity as well as long term survival (Lanzavecchia and Sallusto, Science 2000. 290: 92-97; Champagne et al., Nature 2001. 410: 106-111; Kaech et al., Nat Rev Immunol 2002. 2: 251-262). Enumeration and characterization of antigen-specific T cells is, however, limited by the low frequency of precursors detectable ex vivo and also by the particular readout chosen to identify a T cell as specific for any particular antigen.

The generation of MHC/peptide tetrameric complexes (Altman et al., Science 1996. 274: 94-96), ELISPOT assays (Herr et al., J Immunol Methods 1996. 191: 131-142), intracellular or affinity matrix detection cytokines (Jung et al., J Immunol Methods 1993. 159: 197-207; Manz et al., Proc Natl Acad Sci USA 1995. 92: 1921-1925; Pala et al., J Immunol Methods 2000. 243: 107-124; Mathioudakis et al., J Immunol Methods 2002. 260: 37-42) and more recently quantification with T-cell receptor (TCR) clonotypic probes (Lim et al., J Immunol Methods 2002. 261: 177-194), constitute reliable sensitive approaches for the monitoring of antigen-specific T cells ex vivo (i.e., with limited or no in vitro culture). MHC/peptide tetramers conjugated with fluorochromes allow the detection of epitope-specific T cells based on single cell analysis by flow cytometry. Use of these tetramers has greatly contributed to our understanding of mature T cell differentiation during immune response to pathogens or following vaccination (Murali-Krishna et al., Immunity 1998. 8: 177-187; Pittet et al., Int Immunopharmacol 2001. 1:1235-1247; Klenerman et al., Nat Rev Immunol 2002. 2: 263-272) even though this monitoring has so far been essentially restricted to CD8⁺ T cells. However, as recognition of MHC/peptide complexes by the TCR is degenerate (Mason, Immunol Today 1998. 19: 395-404), the definition of antigen-specific T cells based simply on a stable interaction with these tetramers is questionable (Dutoit et al., J Exp Med, 2002. 196, 207-16).

The capacity of cells to bind tetramers does not imply any particular effector function. For example, detection of anergic specific CD8 T cells has been described in the peripheral blood of patients (Lee et al., Nat Med 1999. 5: 677-685). However, the combination of tetramer staining with detection of intracellular cytokines produced in response to antigen-specific stimulation allows direct visualization of the pattern of cytokines produced by tetramer-binding cells (Appay and Rowland-Jones, J Immunol Methods 2002. 268: 9). In some models, clonal expansion has been shown to tightly regulate the production of cytokines (Bird et al., Immunity 1998. 9: 229-237; Gett and Hodgkin, Proc Natl Acad Sci USA 1998. 95: 9488-9493; Gudmundsdottir et al., J Immunol 1999. 162: 5212-5223) suggesting that the time and duration of stimulation may be critical. These results emphasize the need for combining different methods to accurately identify and quantify the cellular components of an antigen-specific T-cell pool.

Proliferative potential itself constitutes an important parameter for evaluating the differentiation status of antigen-specific T cells. Naive T cells have the capacity to expand and give rise to effector/memory cells (Lanzavecchia and Sallusto, Science 2000. 290: 92-97; Champagne et al., DNA Cell Biol 2001. 20: 745-760; Kaech et al., Nat Rev Immunol 2002. 2: 251-262). T cells that will compose this pool are thought to acquire a high proliferative potential in order to mount a rapid secondary immune response. Other T cells are thought to lose progressively the capacity for clonal expansion after they have terminally differentiated into cells mediating cytokine secretion or killing activity (Sallusto et al., Nature 1999. 401: 708-712; Champagne et al., Nature 2001. 410: 106-111). Quantitative assessment of the proliferative potential during differentiation of mature T cells is, however, still poorly documented.

One method for assaying proliferation utilizes cell labeling with vital fluorescent dyes (Horan and Slezak, Nature 1989. 340: 167-168; Lyons and Parish, J Immunol Methods 1994. 171: 131-137; Wells et al., J Clin Invest 1997. 100: 3173-3183; Allsopp et al., J Immunol Methods 1998. 214: 175-186; Lyons, J Immunol Methods 2000. 1243: 147-154). This assay has been used in various models to track cell division after stimulation either in vitro or, alteratively, in vivo following adoptive transfer (Wallace et al., Cancer Res: 1993. 2358-2367; Gudmundsdottir et al., J Immunol 1999. 162: 5212-5223; Veiga-Fernandes et al., Nat Immunol 2000. 1: 47-53; Champagne et al., Nature 2001. 410: 106-111; Geginat et al., J Exp Med 2001. 194: 1711-9; Kaech and Ahmed, Nat Immunol 2001. 2: 415-422; van Stipdonk et al., Nat Immunol 2001. 2: 423429; Kassiotis et al., Nat Immunol 2002. 3: 244-250; Migueles et al., Nature Immunology 2002. 3, 1061-1068). The equal partition of these fluorescent dyes between daughter cells during cytokinesis allows the use of fluorescence intensity to visualize the successive generations of expanding cells and thus has contributed to a better definition of requirements for T cell expansion. Few groups, however, have taken advantage of the dye dilution to calculate back to the precursor frequency of the proliferating cells in the original T cell population (Wells et al., J Clin Invest 1997. 100: 3173-3183; Givan et al., J Immunol Methods, 1999. 230: 99-112; Song et al., J Immunol 1999. 162: 2467-2471). Indeed, because of the exponential expansion of specific T cells, observation of cells by flow cytometry after several days of culture is misleading; back calculation of precursor frequencies is important to understand fully the distribution of cell types prior to stimulation.

We have previously described a flow dye dilution assay to calculate the precursor frequency and expansion potential of antigen-specific T cells (Givan et al., J Immunol Methods, 1999. 230: 99-112). Precursor frequencies of cells proliferating in response to tetanus toxoid antigen calculated by this dye dilution assay correlated well with, but were about 100-fold higher than, results obtained by the traditional limiting dilution analysis (LDA) using tritiated thymidine. Simultaneous assessment of other T cell functions (for example, cytokine synthesis) might indicate which of these values reflects true antigen-specific proliferation capacity. In addition, it remains to be determined how quantification of antigen-specific T cells by functional assays (cytokine synthesis or proliferation) relates to enumeration of epitope-specific T cells with tetramers of MHC/peptide.

Here we describe a method, based on multiparameter flow cytometry, to visualize simultaneously proliferation and cytokine production by T cells having different capacities to bind MHC/peptide tetramers; this method also allows calculations of the original frequency of these sub-populations of cells ex vivo. Using CD8 T cells from influenza-vaccinated donors, we show that the original CD8 T cell pool can be divided into eight sub-populations (four among tetramer-positive cells and four among tetramer-negative cells) according to the capacity of cells to proliferate and/or to synthesize interferon-γ (IFN-γ) in response to influenza-peptide-pulsed dendritic cells (DC). The precursor frequencies in the original resting population of T cells with different functional capacities were calculated. Our results demonstrate that about half of the tetramer-positive precursors have the capacity both to divide and produce IFN-γ in response to flu peptide. In addition, a similar number (although a much lower proportion) of tetramer-negative cells will proliferate, but these cells will not synthesize IFN-γ.

### MATERIAL AND METHODS

Cells

Cells were isolated by apheresis from HLA A*0201 healthy volunteers, two weeks after immunization with influenza vaccine (Aventis Pasteur, Inc., Swiftwater, Pa.). CD8 lymphocytes were purified by ficoll, cold aggregation (Mentzer et al., Cell Immunol 1986. 101, 312-319), and positive selection with magnetic beads (Miltenyi Biotec, Auburn, Calif.). Autologous DC were differentiated from monocytes with GM-CSF and IL-13 (Goxe et al., Immunol Invest 2000. 29: 319-336). DC and CD8 T cells were frozen in autologous serum with 10% DMSO (Sigma-Aldrich, St Louis, Mo.) and stored in liquid nitrogen until use.

Peptide and Tetramers

M158-66 peptide (GILGFVFTL) derived from the M1 protein of the influenza virus (“flu peptide”) was purchased from Cybergene (St Malo, France) and was >80% pure. Phycoerythrin (PE)-labeled HLA-A*0201/M158-66 tetramers were purchased from Beckman Coulter Immunomics (San Diego, Calif., USA) as were PE-labeled A*0201/HIV gag (SLYNTVATL) tetramers, used as a negative control.

PKH Dye Dilution Assay

Autologous DC were thawed in AIM-V medium (Gibco BRL, France) and loaded overnight with 10 μg/ml flu peptide and 5 μg/ml β2 microglobulin (Sigma) in AIM-V supplemented with 500 IU/ml GM-CSF (Novartis Pharma AG, Basel, Switzerland) and 50 ng/ml IL-13 (Sanofi-Synthelabo, Paris, France). Unloaded DC, used as a control, were left overnight in the same medium without addition of flu peptide. On the day of the assay, CD8 T cells were thawed in AIM-V in the presence of 5 IU/ml of DNase (Gibco BRL). Cells were incubated for 5 min at 37° C. and then washed twice in AIM-V medium. Cells were then stained with PKH67 fluorescent dye (Green Fluorescent Cell Linker Kit, Sigma). Briefly, cells were resuspended in “Diluent C” at 2×10⁷ cells/ml, mixed immediately with an equal volume of PKH67 dye solution (4 μM) and incubated for 3 min at room temperature. The staining reaction was stopped by addition of an equal volume of human AB serum (Biowhittaker, Walkersville, Md., USA) followed by a wash step in AIM-V medium supplemented with 5% AB serum (“complete medium”). The PKH67-labeled cells were resuspended in complete medium and plated in 12- or 24-well plates (5×10⁶ cells/well or 2.75×10⁶ cells/well, respectively). The peptide-loaded or unloaded DC were washed twice, resuspended in complete medium, and added to the CD8 cells at a ratio of 1 DC for 5 CD8 T cells. After 6 days of culture, cells were stained with tetramers and antibodies for analysis by flow cytometry, as described below.

Elispot for IFN-γ

Multiscreen nitrocellulose 96-well plates (Millipore, Bedford, Mass.) were coated with 10 μg/ml of monoclonal antibody (mAb) specific for IFN-γ (1-D1K, Mabtech, Stockholm, Sweden) for 1 hour at 37° C. After blocking the wells with AIM-V supplemented with 10% AB serum (1 hour, 37° C.), CD8 T cells (2×10⁵ to 1×10² cells/well) were seeded in triplicate and stimulated with flu peptide-loaded or unloaded DC 5×10⁴ DC/well). Soluble anti-CD3 mAb (HIT3a, Pharmingen, France) added at 50 ng/ml was used as a positive control for stimulation of CD8 T cells (10⁵/well). Plates were incubated overnight at 37° C. in 5% CO₂, washed, and then incubated with biotinylated anti-IFN-γ mAb (2 μg/ml; 7-B6-1; Mabtech). After 2 hours incubation, the plates were washed, stained for 1 hour with Vectastain Elite Kit (Ab Cys, Paris, France), and revealed with aminoethyl carbazol at 1 mg/ml in 50 mM acetate buffer with 0.015% H₂O₂ (all from Sigma). Counting of spot-forming cells was performed using a computer-assisted microscope (Carl Zeiss, Le Pecq, France). Secretion of IFN-γ was considered positive when the number of spots in the triplicates with flu-peptide-loaded DC was significantly different from the number of spots in the triplicates with unloaded DC (student t test, p<0.05).

Tetramer and CD8 Staining

CD8 T cells (8×10⁵ cells) were stained with tetramers, either prior to culture (for ex vivo determination) or after PKH67 staining and subsequent culture for 6 days. Cells were mixed with PE-tetramers in flow buffer (phosphate-buffered saline with 5% fetal bovine serum and 0.1% sodium azide) for 20 min at 37° C., followed by incubation for 15 min at 4° C. with anti-CDS mAb conjugated with fluorescein (clone B9.11, Beckman Coulter Immunotech, Marseille, France) or PerCP (clone SK1, Becton Dickinson, San Jose, Calif.), or isotype controls. Cells were washed and resuspended in flow buffer containing 3 nM TO-PRO-3 (Molecular Probes, Leiden, The Netherlands) for immediate acquisition of data by flow cytometry. Alternatively, the cells were further stained for detection of intracellular IFN-γ.

Detection of Intracellular IFN-γ in PKH-Labeled T Cells

Cells labeled with PKH67 were cultured for 6 days with unloaded DC or DC loaded with flu-peptide. Duplicate wells received re-stimulation on day 5 by a second addition of unloaded or flu-peptide-loaded DC for the final 16 hours of culture. Brefeldin A (1 μg/ml; Sigma) was added to all wells for the final 16 hours. Some cultured cells were stimulated with PMA (10 ng/ml, Sigma) and ionomycin (500 ng/ml, Sigma) as a positive control for IFN-γ synthesis. On day 6, cells were harvested, stained with tetramers and cell surface markers in flow buffer containing 1 μg/ml brefeldin A, and then were fixed in 4% formaldehyde (Sigma) for 20 minutes. After fixation, cells were washed three times in flow buffer containing 0.1% saponin (Sigma). Cells were then stained for 1 hour at 4° C. with human IgG block (60 μg/8×10⁵ cells; Sigma) and allophycocyanin (APC)-conjugated anti-IFN-γ (20 ng/8×10⁵ cells, clone B27) or IgG1 isotype control (clone MOPC 21) from Becton Dickinson (San Jose, Calif.). Cells were then washed two times in flow buffer with 0.1% saponin and a final time in flow buffer. They were then resuspended in 1% formaldehyde (Sigma) and assayed on the flow cytometer the following day.

Flow Cytometry and Data Analysis

Data from cells (80,000-500,000 live cells) were acquired on a Becton Dickinson (San Jose, Calif.) FACSCalibur flow cytometer, with two lasers (488 nm and 635 nm) and four fluorescence photomultiplier tubes with filters appropriate for PKH 67, phycoerythrin, PerCP, and allophycocyanin or TO-PRO-3. After acquisition of the data into list mode files, cells were gated by their forward and side scatter characteristics, so as to exclude dead cells (with low forward scatter) but to include both resting and activated lymphocytes (with high forward scatter) for further analysis. For analysis of proliferation, tetramer staining, and cytokine synthesis, cells were also gated on CD8-positivity. For phenotyping and for determination of the proportion of cells synthesizing cytokine and binding tetramers, data were analyzed using CellQuest software (Becton Dickinson, San Jose, Calif.). For proliferation and precursor frequency analysis, ModFit software version 3.1 (Verity Software House, Topsham, Me.) was used to analyze PKH fluorescence and to calculate the precursor frequencies of cells gated on cytokine and on tetramer fluorescence. For the gated cells, the software examines the PKH67 fluorescence intensity distribution, derives Gaussian curves centered on halving intensity values from the original parental intensity, and calculates how many cells at the beginning of the culture period (the precursor cells) were required to account for the distribution of proliferating cells at the time of the assay (see Wells et al., J Clin Invest 1997. 100: 3173-3183; Givan et al., J Immunol Methods, 1999. 230: 99-112; Song et al., J Immunol 1999. 162: 2467-2471). The number of decades for the logarithmic scale was calculated with calibrated beads (Spherotech, Libertyville, Ill.). Cells having undergone two or more divisions were included in the proliferative fraction. Precursor frequencies of the proliferating and non-proliferating cells in each of four populations (double negative, cytokine-positive, tetramer-positive, and double positive) were determined. The percent of non-proliferating cells was then applied to the number of gated cells in the data file to give the number of cells before culture that will not proliferate. Knowing the number of cells that was destined not to divide and also knowing the precursor frequency of the proliferating cells, the number of cells destined to divide could be calculated. This same set of calculations was applied to the four gated populations of cells (according to their tetramer-binding and cytokine production), resulting in a description of the proliferative potential of the resting cells as they existed at time zero. The number of cells in the eight sub-populations was then summed so that all precursor results could be expressed as percent of the total original, resting population.

For multiparameter precursor frequency (PF) analysis, the following formulae were used to calculate back to the cells in the original, resting culture:
number_{non-proliferating cells in the initial population}=[(proportion_{cells that have not proliferated and that are present in the sample at tic end of the experiment})+(0.5*proportion_{cells that have divided only once and that are present in the sample at the end of the experiment})]*[number_{gated cells in data file}],
and
number_{proliferating cells in the initial population}=[(PF_{proliferating cells})*(number_{non-proliferating cells in the initial population})]/[1−PF_{proliferating cells}],
or otherwise expressed in the condition of the experience
number_{non-prolif cells (day 0)}=[(proportion_{parental generation (day 6)})+(0.5*proportion_{generation 2 (day 6).})]*[number_{gated cells in data file}]
and
number_{prolif cells (day 0)}=[(PF_{prolif cells})*(number_{non-prolif cells (day 0)})]/[1−PF_{prolif cells}]

### RESULTS

Frequency of Flu-Specific CD8⁺ T Cells Determined by Tetramer Binding, The Elispot Assay for IFN-γ and the Dye Dilution Assay

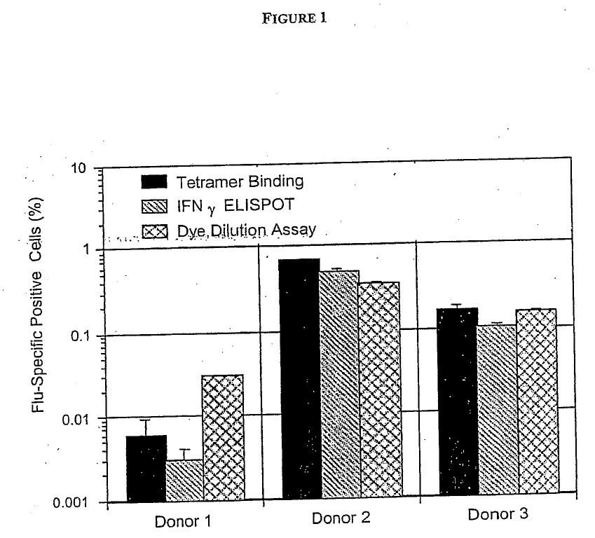

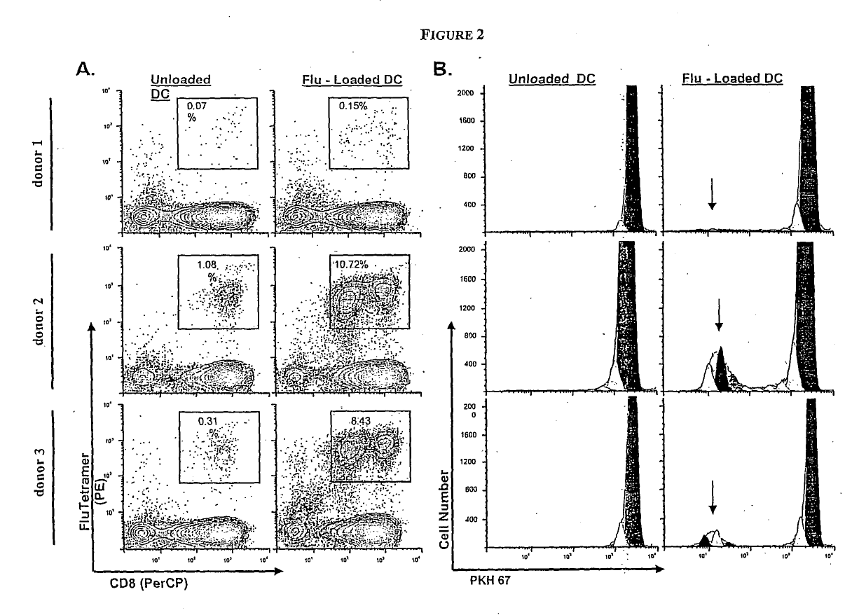

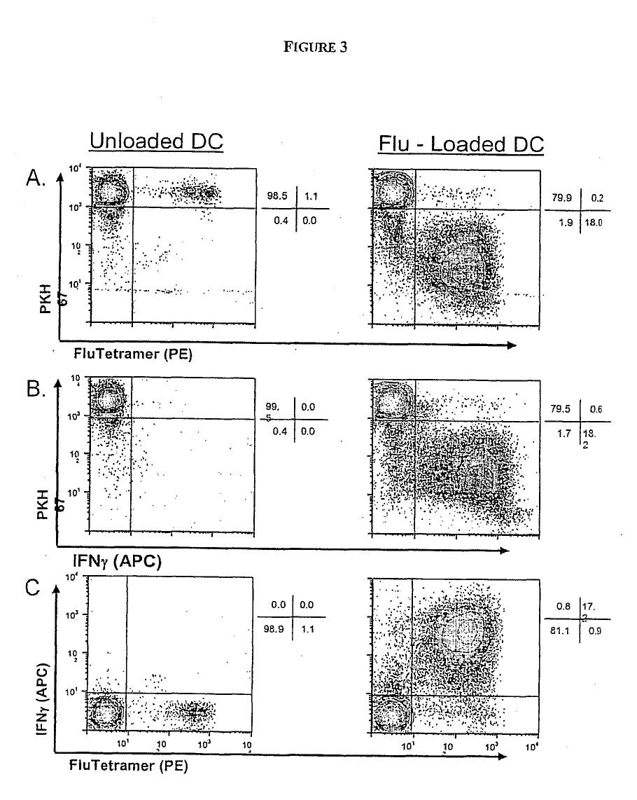

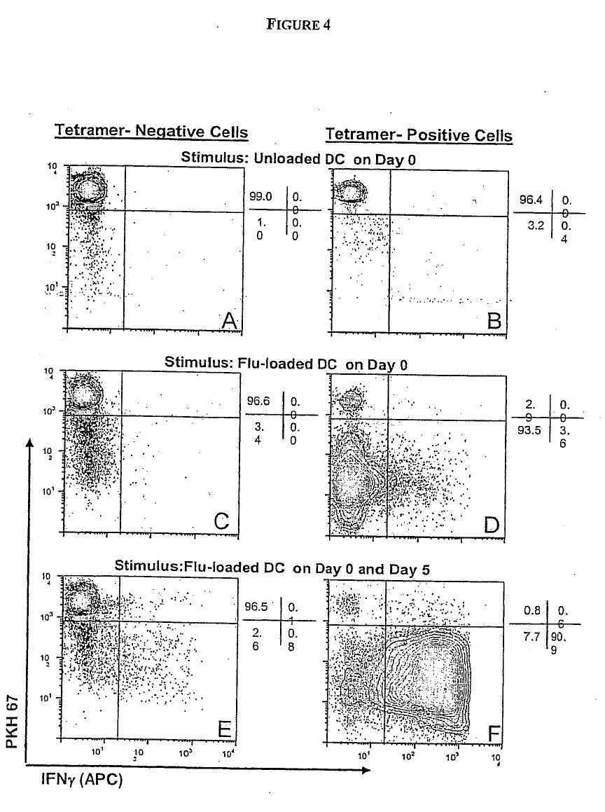

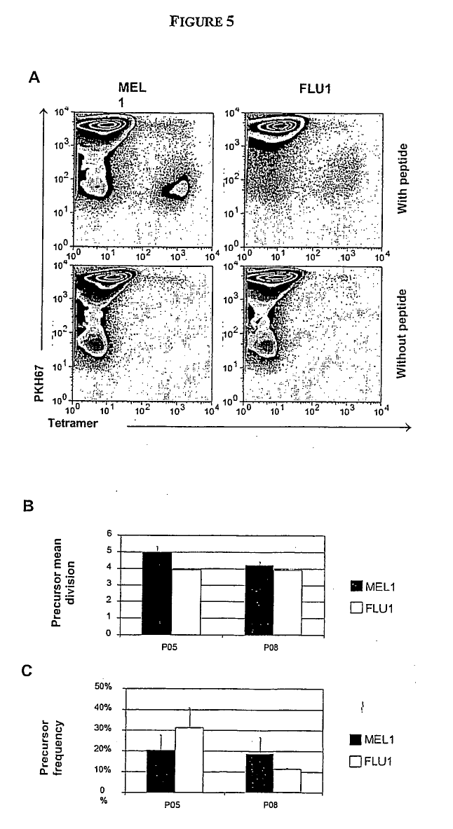

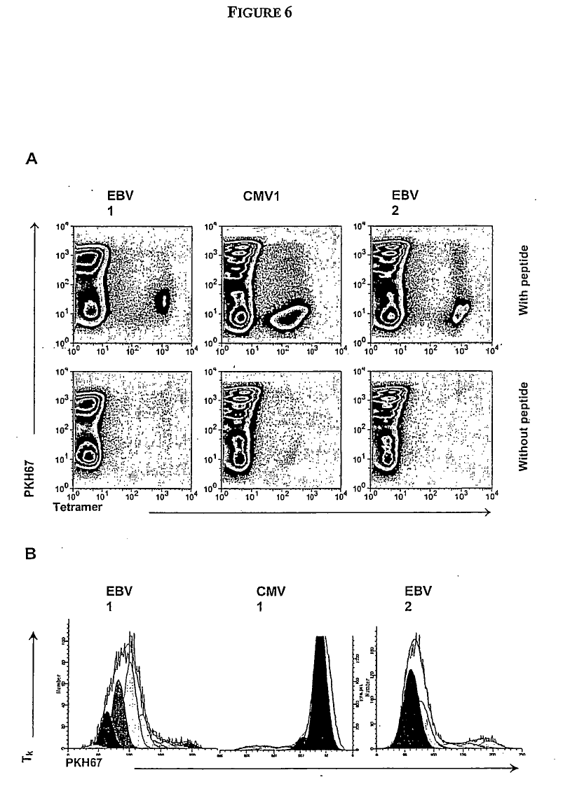

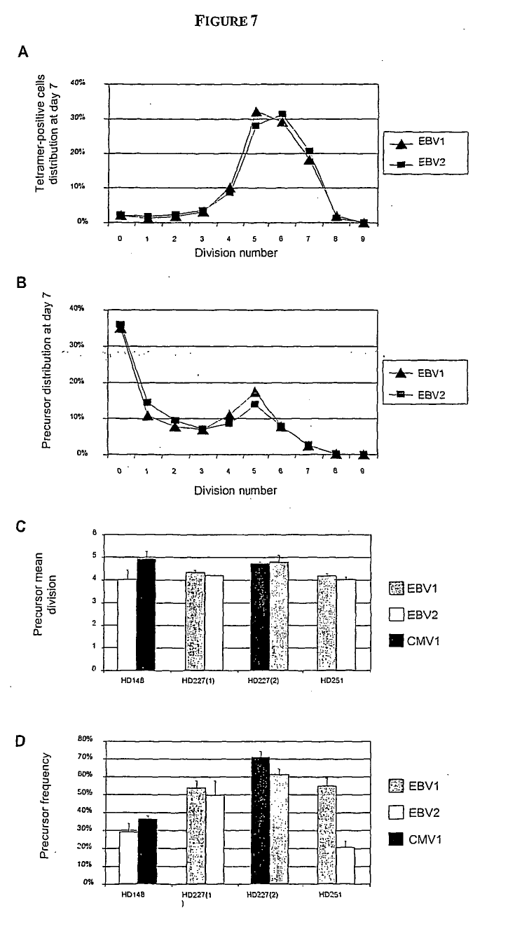

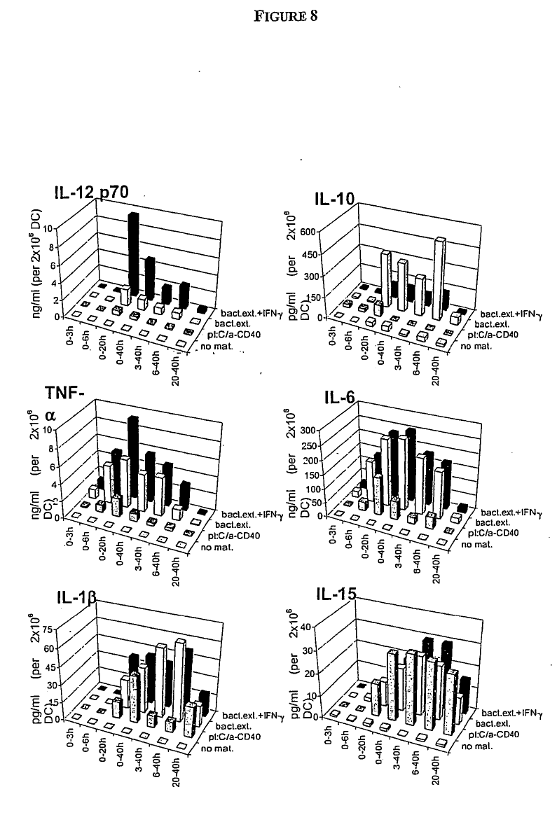

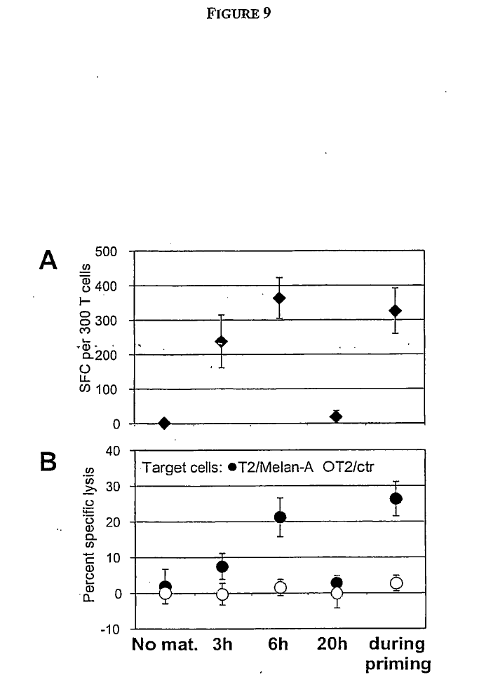

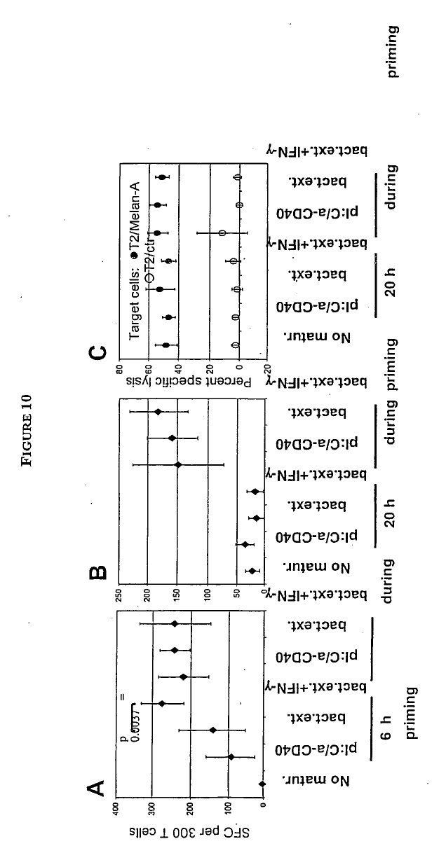

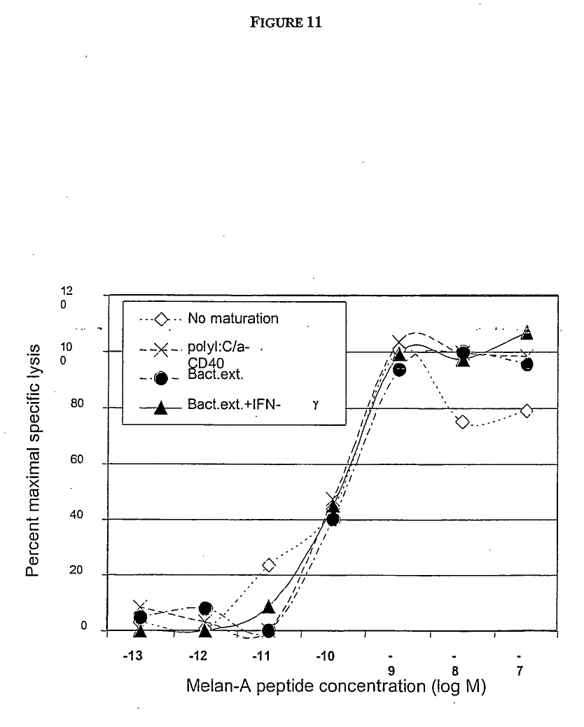

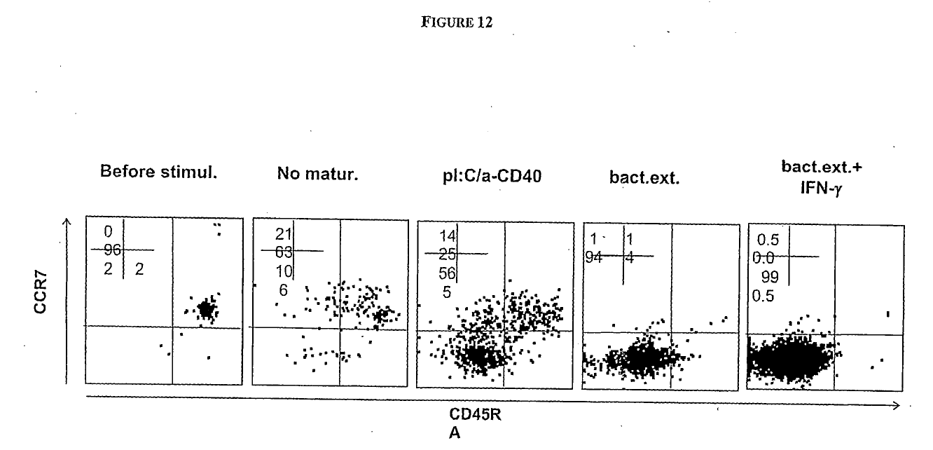

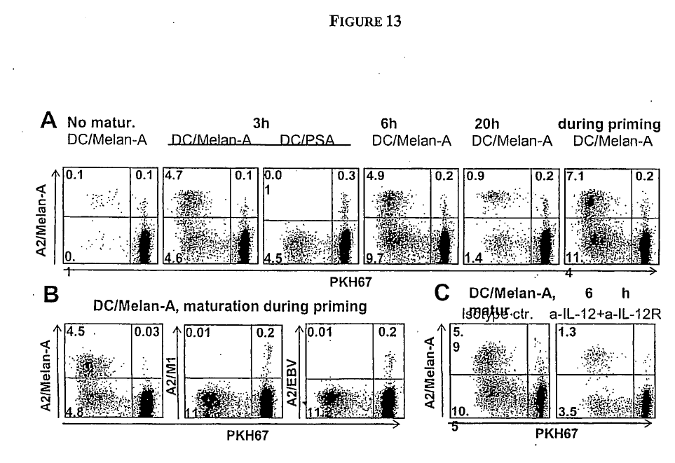

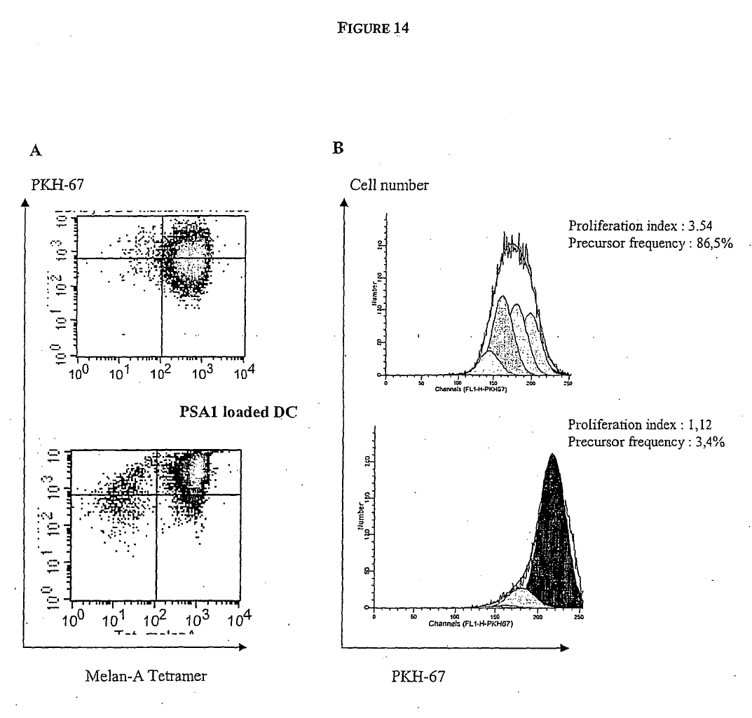

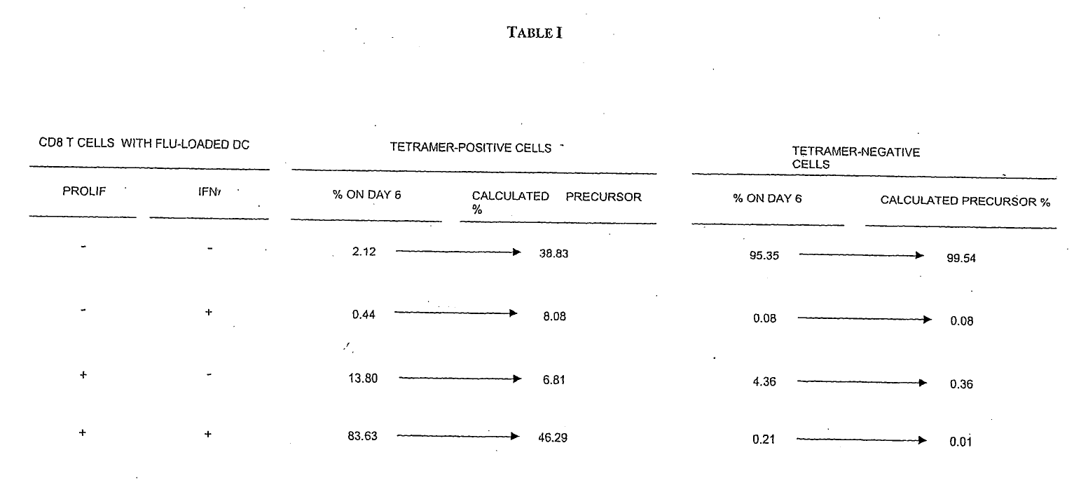

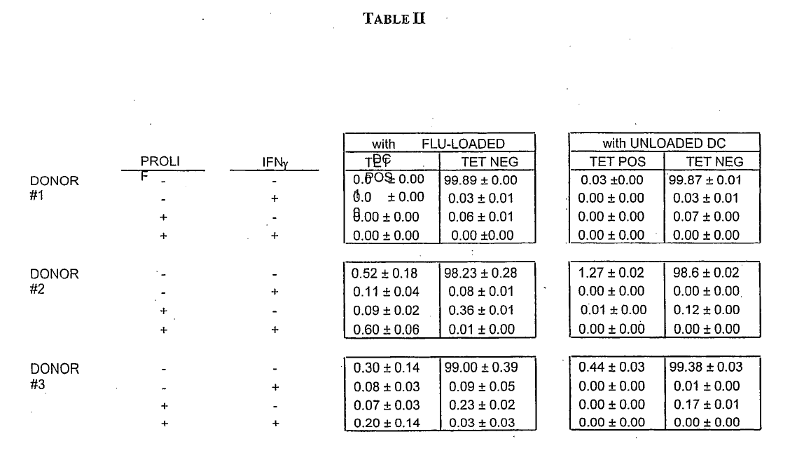

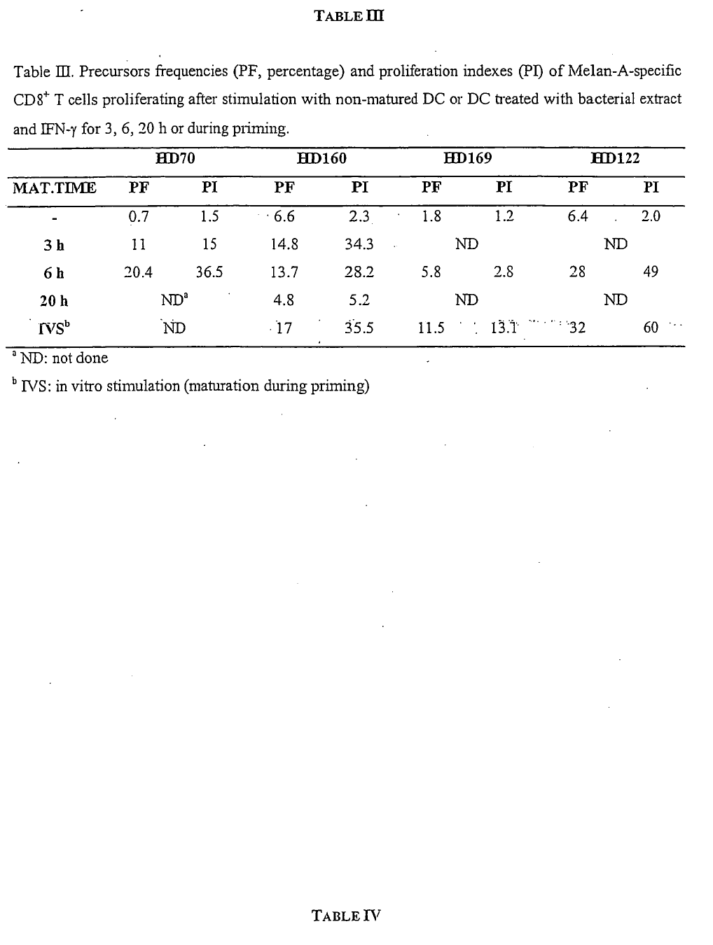

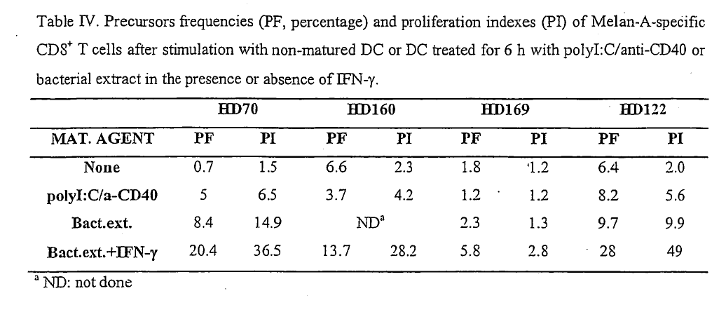

The frequency of antigen-specific CD8⁺ T cells ex-vivo was first estimated using three different independent methods: staining with MHC/peptide tetrameric complexes, the ELISPOT assay for IFN-γ production, and the flow dye dilution assay for proliferation precursors. Assessment of CD8⁺ T cells specific for the M1₅₈₋₆₆ influenza epitope was chosen as a model system for antigen specificity. Three HLA-A2 individuals were vaccinated against influenza virus and circulating lymphocytes were collected two weeks later. CD8⁺ T cells Were purified and stained with tetramers to determine the ex vivo frequency of cells specific for flu peptide. As shown in FIG. 1, the frequency of HLA-A*0201/M1₅₈₋₆₆ positive cells (TET⁺) represented 0.713+/−1-0.005% and 0.170+/−0.022% of CD8⁺ T cells in donor 2 and 3, respectively. In donor 1, TET⁺ CD8 cells were also detected but at a much lower frequency: although this population represented only 0.006+/−0.003% of the CD8⁺ cells, it appeared as a bright cluster not seen in the HIV gag control.

Specific CD8 T cells were then characterized by a second method based on the proportion of cells secreting cytokines in response to antigenic stimulation. We chose to do this quantification by IFN-γ ELISPOT, because it is widely used to monitor immune responses in blood specimens. CD8⁺ T cells were stimulated for 18 hours with flu-peptide-pulsed DC. For all donors, the frequencies of flu-specific IFN-γ-secreting T cells were similar to the frequencies obtained by tetramer staining (FIG. 1). For donor 1, the frequency was much lower than for donors 2 and 3, but significantly different from controls using unloaded DC (p=0.02).

We next used the flow dye dilution method (Wells et al., 1997; Givan et al., 1999; Song et al., 1999) to estimate the precursor frequency of flu-specific CD8⁺ T cells ex vivo, according to their capacity to proliferate in response to peptide stimulation in vitro. Purified CD8⁺ T cells were labeled with PKH67 fluorescent dye and cultured with DC loaded with flu peptide. Stimulation with flu-loaded DC led to an increase in TET⁺ cells; therefore they represented a substantial sub-population in donors 2 and 3 after 6 days of culture (FIG. 2A). From the PKH intensity distributions (FIG. 2B), the frequencies of CD8⁺ T cells originally present at day 0 with the capacity to proliferate in response to the flu peptide were calculated. In all three donors, we detected a specific expansion in response to flu peptide-loaded DC (FIG. 2B; see arrows). The proliferation precursor frequencies calculated were similar to those determined by IFN-γ ELISPOT and tetramer binding (FIG. 1).

Combination of Dye Dilution Assay with Tetramer Staining and Detection of IFN-γ Synthesis to Analyze Simultaneously Different Functional Responses Mediated by Individual CD8 T Cells.

In order to determine how proliferation correlates with cytokine secretion, we combined the dye dilution assay with intracellular detection of IFN-γ synthesis. In addition, cells were stained with tetramers. CD8 cells labeled with PKH67 were stimulated for 6 days with flu-peptide-pulsed DC. Preliminary experiments showed that, in the absence of re-stimulation at the end of the culture period, only weak IFN-γ synthesis was detectable (see FIG. 4D below). Therefore we performed a 16 hour re-stimulation with flu-peptide-pulsed DC just before harvesting the cells on day 6. After culture, the cells were stained with tetramers and with anti-CD8 antibody, followed by fixation, permeabilization, and staining for IFN-γ.

A representative example of the PKH fluorescence profiles and tetramer staining is given in FIG. 3A. After a 6 day-stimulation with flu-peptide-loaded DC, a large majority of tetramer-positive cells displayed low PKH fluorescence intensity indicating that they were expanded T cells (PROLIF⁺). However, we detected a small population among the TET⁺ cells that had not proliferated (1.0% for donor 2 in FIG. 3A). Analysis of IFN-γ secretion together with PKH67 fluorescence showed that IFN-γ was synthesized specifically after stimulation with flu-peptide-loaded DC and the vast majority of these IFN-γ⁺ cells corresponded to expanded T cells (97% for donor 2 as illustrated in FIG. 3B). No IFN-γ synthesis was detected when cells were cultured from day 0 with unloaded DC. Interestingly, this analysis revealed the presence of a small population of PROLIF⁺ cells which had not synthesized IFN-γ_{.} (8.5%) in response to flu-peptide stimulation. Such IFN-γ⁻ cells were specific in that they were present in much lower numbers in cultures using unloaded DC. We next plotted tetramer staining versus IFN-γ synthesis (FIG. 3C). The vast majority of TET⁺ cells synthesized IFN-γ_{.} in response to flu-peptide stimulation (95% for donor 2 in FIG. 3C). However, a small population of TET⁺ cells (5%) did not synthesize detectable amounts of IFN-γ. There was also a clear population of TET⁻ cells that made IFN-γ (1% for donor 2).

The combination of tetramer staining with proliferation and cytokine detection offers the opportunity to analyze among TET⁺ cells, the relative proportion of cells that are recruited to proliferate and synthesize IFN-γ in response to antigenic stimulation. Similarly, recruitment to proliferate and to synthesize cytokines can be analyzed in the same sample on TET⁻ cells. An example of the profiles of TET⁺ and TET⁻ cells obtained for donor 2 are presented in FIG. 4. TET⁺ cells were composed mainly of PROLIF⁺ cells on day 6 following a single stimulation with flu-peptide-loaded DC on day 0 (FIG. 4D). However, on day 6, the assay revealed a small fraction of TET⁺ cells (2.9% for donor 2) that had not proliferated.

Analysis of cytokine production revealed that IFN-γ⁺ cells were mainly restricted to TET⁺ cells that had expanded in response to flu-peptide-loaded DC (FIG. 4F) even though cytokine production was barely detectable without a short re-stimulation on day 5 (FIG. 4D). There was also a population of PROLIF⁺ TET⁺ cells that did not make cytokine. Thus, four subsets of tetramer-positive cells could be identified (FIG. 4F): (1) cells that had been recruited to expand and that had synthesized IFN-γ (TET⁺ PROLIF⁺ IFN-γ⁺), (2) cells that had expanded but had not synthesized IFN-γ (TET⁺ PROLIF⁺ IFN-γ⁻), (3) cells that had not proliferated but had synthesized IFN-γ (TET⁺ PROLIF⁻ IFN-γ⁺), and finally, (4) cells that had not proliferated and had not synthesized IFN-γ although stained with tetramers (TET⁺ PROLIF⁻ IFN-γ⁻).

All four of these sub-populations could also be detected amongst the TET⁻ T cells, but in different proportions. Most of the TET⁻ cells (96.6% for donor 2 as seen in FIG. 4E) had not proliferated; however, interestingly, a small fraction had proliferated. By contrast with the TET⁺ population, the expanded TET⁻ cells were predominantly unable to synthesize IFN-γ. Similar results were obtained with cells from all donors (see table II below).

Altogether, these data show that all of the theoretical eight populations can be identified when proliferation, cytokine production and tetramer staining are analyzed together. Importantly, the CD8⁺ T cell population responding to flu-peptide-loaded DC included both TET⁺ and TET⁻ cells.

Precursor Frequencies Calculated from Multiparameter Flow Cytometric Analysis of Stimulated CD8⁺ T Cells.

Since T cells that proliferate in response to flu-peptide-pulsed DC expand during culture, their absolute numbers increase during the culture period compared to the ex vivo situation. Thus, these populations are over-represented at the end of the culture compared to the cells that did not proliferate. Indeed, the degree of this over-representation will depend on the number of re-stimulations and on the length of the culture period. Additional calculations are therefore required to give the true picture of the original proportion (that is, the precursor frequencies) of individual CD8⁺ T cell subsets in the resting population.

For each subset (for example TET⁺ IFN-γ⁺), we calculated the precursor frequency of the proliferating cells from the PKH profile of the gated cell population. An example of precursor frequencies calculated back from the percentage of individual gated populations at day 6 is shown in Table I. In this table, the importance of back calculation is demonstrated: on day 6, while almost all the tetramer-positive cells were proliferating (97.4%), the calculated precursor frequencies indicate that only half of the initial TET⁺ cells (53.1%) had actually been stimulated to expand by exposure to antigen.

Table II presents the precursor frequencies of TET⁻ and TET⁺ T-cell subsets calculated for the three donors when cells had been cultured with flu-peptide-loaded DC or unloaded DC. The values for each culture condition represent the combined percents of TET⁻ and TET⁺ CD8 precursors; they add up to 100% of the cells present in the original sample.

The multiparameter analysis for donors 2 and 3 revealed that 54-60% of TET⁺ precursors proliferated and/or produced IFN-γ in response to peptide stimulation (table II). However, approximately half of the TET⁺ cells were non-responsive according to these two functional criteria. Among the responding TET⁺ precursors, the majority both proliferated and made IFN-γ. In contrast, the response of the TET cells was mainly just proliferation, without cytokine synthesis. It should be noted that, in contrast to the TET⁺ cells, some of the TET⁻ precursors proliferated even with unloaded DC. Calculations from donors 2 and 3 reveal that similar numbers of TET⁺ and TET⁻ cells responded to flu peptide stimulation (PROLIF⁺ and/or IFN-γ⁺). Overall, although only a small fraction of the TET⁻ cells responded to peptide, they were a substantial proportion of the total number of responding cells. In donor 1, no responses to peptide stimulation were seen in excess of the mainly TET cells which responded in the absence of peptide stimulation.

### DISCUSSION

The functions of T cells are diverse and, therefore, one assay may not be sufficient to reveal all precursors able to contribute to an antigen-specific response. Here, by using triple parameter analysis by flow cytometry, we have been able to identify eight subsets and to quantify their relative proportions in the original resting population.

Tetramer technology and the ELISPOT assay are two current methods for estimating the frequency of antigen-specific T cells, based on different properties of the cells (see Bercovici et al., Clin Diagn Lab Immunol 2000. 7, 859-64; Pittet et al., Int Immunopharmacol 2001. 1: 1235-1247). Here we have also made use of the flow dye dilution method to calculate precursor frequencies based on the capacity of T cells to proliferate (Wells et al., J Clin Invest 1997. 100: 3173-3183; Givan et al., J Immunol Methods, 1999. 230: 99-112; Song et al., J Immunol 1999. 162: 2467-2471). In this dye dilution method, culturing stained cells with antigen stimulation allowed examination of proliferation as another functional parameter. It also provided the benefit of sub-population expansion to increase the sensitivity for detecting rare responding cells and for calculating the precursor frequencies of sub-populations in the original mixture of cells. We have shown here that, although the three independent methods assay different properties of T cells, the number of cells positive by the three assays were similar for the donors tested. Although giving similar results, these assays may reveal distinct sub-populations of T cells. For example, it has been described that similar T cell responses can be achieved by T cell clones having different capacity to secrete IFN-γ and to bind MHC/peptide tetramers (Rubio-Godoy et al., Proc Natl Acad Sci USA 2001. 98, 10302-7).

By substituting a flow cytometric method detecting internal cytokine staining for the ELISPOT method, we were able to measure tetramer binding, cytokine synthesis, and proliferation simultaneously and analyze all three parameters on a single cell basis. In this three-parameter assay, not all cells are positive for all three characteristics; the original population can, in fact, be divided into eight subsets with respect to these three parameters. Because the method proposed here involves calculation of precursor frequencies, we have been able to show that, although a high proportion of the tetramer-binding cells and a low proportion of the tetramer-negative cells respond to flu-peptide stimulation, the responding cells are approximately equally represented among the tetramer-binding and tetramer-negative populations. However, only the tetramer-positive cells both proliferate and secrete IFN-γ. By contrast, the tetramer-negative cells that proliferate in general do not synthesize IFN-γ. These TET⁻ cells that proliferate may correspond to a distinct subset of the whole TET⁻ population. Alternatively, they could represent a subset of TET⁺ cells that have low TCR avidity or have down-modulated their TCR expression after peptide stimulation and additionally do not synthesize IFN-γ. Equally important is that half of the tetramer-positive cells do not either proliferate or synthesize IFN-γ in response to flu peptide. It is possible that these cells may respond in other ways or they may not be functional at all. This could be studied by adding additional parameters to the current assay.

We identified some rare precursors that will be able to produce IFN-γ although they will not expand; the method described here shows that this sub-population has tetramer-positive and tetramer-negative cells in similar numbers. These results suggest that some CD8 T cells do not require clonal expansion to produce IFN-γ. We have also found that some cells will proliferate but do not make IFN-γ. The tetramer-negative cells that proliferate are mainly in this population. This subset could represent a fraction of responsive T cell precursors with little or no affinity for the specific peptide. Our method could be used to compare the frequency of these precursors in various populations of effector/memory T cells (Sallusto et al., Nature 1999. 401: 708-712). Moreover, the method could also be used to compare the ability of antigen-presenting cells like dendritic cells to trigger T-cell activation and differentiation.

The difficulty in evaluating the repertoire of T cells, naive or experienced, that can potentially respond to a given antigen relates to the diversity of the T cell clones, to the low frequency of any specific clone, and to the pattern of effector functions shaped by previous antigenic challenge. Although different sensitive assays have been used to detect these rare antigen-specific T cells, no single assay can integrate this complexity in order to describe the diversity of the antigen-specific T-cell pool. In the present work, we have used a new single cell, multiparameter method combined with precursor frequency analysis to evaluate the relative frequencies of sub-populations with different potential responses within a mixed population of cells. The culture of cells allows the use of proliferation as a functional parameter and, in addition, can facilitate detection of rare responders. Because the method involves the calculation of precursor frequencies, it is not biased by the length of culture time nor by the expansion of certain populations. With the capabilities of new, multiparameter flow cytometers, the method could be extended to include additional markers of T-cell function (e.g. cytokines, chemokines, perforin/granzyme), activation (e.g. CD25, CD69), and differentiation or migration (e.g. CD27, CD28 CD62L, CCR7). Many of these parameters or others could be combined into a description of the complex response potential of a T-cell population.

### EXAMPLE 2

The present work compares different populations of memory CD8 T cells, one specific for a viral epitope and one specific for the differentiation antigen MART1, in their capacity to proliferate, in order to identify potential differences between viral and tumor specific CD8 T cell populations.

### MATERIAL AND METHODS

Peptides and Tetramers

Peptides presented by HLA-A*0201 molecules were used: Melan-A MART1 (ELAGIGILTV 26-35 (27L), Neosystem) and influenza matrix protein (GILGFVFTL 58-66, Cybergene). Phycoerythrin (PE)-labeled HLA-A*0201 tetramers contained the following peptides: Melan-A 26-35(27L) and influenza matrix protein 58-66 and were purchased from Proimmune, (Oxford, GB), and Beckman Coulter Immunomics (San Diego, Calif.) as were PE-labeled A*0201/HIV gag (SLYNTVATL) tetramer, used as a negative control.

Patients Samples

Aphaeresis were collected from HLA-A*0201 melanoma patients included in phase I/II clinical trial.

Dendritic Cell Differentiation

Dendritic Cells (DC) were differentiated using the VacCell processor (IDM, Paris, France) as previously described (Goxe et al., Immunol Invest 2000. 29: 319-336). Briefly, peripheral blood mononuclear cells (PBMC) were differentiated from monocytes with a 7 days in vitro culture in serum-free IDM VacCell medium (Life Technologies, Paisley, GB) supplemented with 500 U/ml GM-CSF (Novartis Pharma AG, Basel, Switzer-land) and 50 ng/ml IL-13 (Sanofi-Synthelabo, Paris, France). DC were then isolated by elutriation. Purity ranged from 80% to 99%; viability was above 95%. DC were frozen in 4% human albumin with 10% DMSO (Sigma Aldrich, St Louis, Mo.) and stored in liquid nitrogen.

CD8 T Cell Isolation

CD8⁺ cells were purified from PBMC by positive selection using CD8⁺ Microbeads (Miltenyi Biotec, Paris, France) according to manufacturer's instructions. Purity determined by flow cytometry was above 90% CD3⁺ CD8⁺ among alive cells. CD8 T lymphocytes were frozen in FCS with 10% DMSO and stored in liquid nitrogen until use.

Tetramer Staining and Immune Phenotyping

Staining for analysis by flow cytometry was performed in FACs buffer (PBS containing 2% FCS and 0.2% NaN₃). Cells were incubated with tetramers for 20 minutes at 37° C., then for 15 minutes at 4° C. with the following monoclonal antibodies: anti-CD3-APC (UCHT-1, Immunotech, Marseille, France), anti-CD4 conjugated with FITC (13B8.2, Immunotech) or -PerCP (SK3, Becton Dickinson, San Jose, Calif.), anti-CD8 conjugated with FITC, -PE (B9.11, Immunotech), or PerCP (SK1, Becton Dickinson), anti-CD14-APC (RMO2, Immunotech), anti-CD19-APC (J4.119, Immunotech), anti-CD56-APC (N901, Immunotech) or matched isotype controls. After washing, cells were incubated for 15 min at 4° C. with a goat anti-mouse-IgG1 FITC mAb (Southern Biotechnology Associates, Birmingham, Ala.), washed and stained for an additional 15 min at 4° C. with anti-CD8 PerCP or isotype control. Cells were resuspended in FACs buffer after final washing and stained with 3 nM TO-PRO-3 (Molecular Probes, Leiden, the Netherlands) when cells were not stained with antibodies coupled to Allophycocyanin. At least 100,000 viable CD8⁺ events were acquired on a FACscalibur with CellQuestPro software (Becton Dickinson). The specificity of tetramer staining was controlled with an irrelevant tetramer. Cells stained by HIV gag tetramer represented always less than 0.02% of total alive CD8⁺ cells.

PKH Dilution Assay

Dendritic cells were thawed in AIMV supplemented with 1% P/S and pulsed with the appropriate peptide (10 μg/ml) and β2-microglobuline (5 μg/ml, Sigma) or in absence of any additive. After loading overnight, DC were treated for 30 minutes at 37° C. with 50 μg/ml mitomycine C (Sigma, St. Louis, Mo.) and washed twice carefully. Purified CD8+ cells were labeled with PKH67 (Sigma, St. Louis, Mo.) according the manufacturer's instructions. Labeled cells were cultured with unloaded or peptide-pulsed DC in presence of exogenous cytokines (IL-2, 10 U/ml and IL-7, 5 ng/ml). On day 7, CD8⁺ cells were harvested, and stained with tetramers, anti-CD8 antibodies and TOPRO3, and analyzed by flow cytometry. The precursor frequencies (PF) and precursor mean divisions (PMD) among viable tetramer⁺ CD8⁺ cells were calculated with ModFit software (Verity Software House, Topsham, Me.), according to the following formulas:

$$PF = \frac{{\sum }_{k ⩾ 2}^{} \frac{{T}_{k}}{{2}^{k}}}{{\sum }_{k ⩾ 0}^{} \frac{{T}_{k}}{{2}^{k}}} ; PMD = \frac{{\sum }_{k ⩾ 2}^{} \frac{{T}_{k}}{{2}^{k}} k}{{\sum }_{k ⩾ 2}^{} \frac{{T}_{k}}{{2}^{k}}}$$

where k represents the number of divisions accomplished at day 7, and Tₖ the number of tetramer positive cells detected in the generation k.

### RESULTS

As cell proliferation increases the frequency of rare proliferating precursors, we could characterize the proliferation capacity of MEL1 specific CD8 T cells in patient 08 and in another patient P05, although MEL1 specific CD8 cells were undetectable ex vitro with tetramers in this patient. In parallel, we have followed the proliferation of FLU1 specific CD8 T cells detectable in both patients. The proliferation profile at day 7 of MEL1 and FLU1 specific CD8 T cells in patient P05 is shown in FIG. 5A. MEL1 specific CD8 T cells expanded well in response to peptide-loaded DC as shown by the high frequency of tetramer positive cells at day 7, and decreased PKH fluorescence intensity (FIG. 5A). The mean division number of precursors in the two melanoma patients was about 4 to 5 for MEL1 and FLU1 specific CD8 T cells (FIG. 5B). Similar proportion of precursors were recruited to proliferate in both pools of memory CD8 cells (10% to 30% of initial specific CD8 cells, FIG. 5C). Altogether, these data show that MEL1 specific CD8 T cells that have been differentiated in vivo during disease progression can be recruited and proliferate in vitro as well as CD8 T cells specific for viral antigens.

### EXAMPLE 3

The present work compares different populations of memory CD8 T cells specific for viral epitopes in their capacity to proliferate. We asked whether particular T cell subsets can be identified.

### MATERIAL AND METHODS

The experiments were carried out as described in example 2

Peptides and Tetramers

Different peptides, presented by HLA-A*0201 molecules, were used: CMV pp65 (NLVPMVATV, 495-503, Neosystem, Strasbourg, France); EBV BMLF1 (GLCTLVAML 280-288, Cybergene, Huddinge, Sweden) and EBV LMP2a (CLGGLLTMV 426-434, Cybergene). Phycoerythrin (PE)-labeled HLA-A*0201 tetramers contained the following peptides: EBV BMFL1 280-288, EBV LMP2 426-434 and CMV pp65 495-503 and were purchased from Proimmune, (Oxford, GB), and Beckman Coulter Immunomics (San Diego, Calif.) as were PE-labeled A*0201/HIV gag (SLYNTVATL) tetramer, used as a negative control.

Healthy Volunteers

Aphaeresis were collected from HLA-A*0201 healthy donors

Dendritic Cell Differentiation

The dendritic cells were obtained as described in example 2

CD8 T Cell Isolation

The CD8 T cells were obtained as described in example 2

Tetramer Staining and Immune Phenotyping

The tetramer staining and immune phenotyping were carried out as described in example 2.

PKH Dilution Assay

The PKH dilution assay and the different calculus were carried out as described in example 2.

### RESULTS

As memory CD8 cells may persist with different capacity to expand following antigenic stimulation, we compared the proliferation potential of three epitope-specific populations. CD8⁺ T cells were purified from healthy volunteers, labeled with the viable PKH67 fluorescent dye and stimulated in vitro with unloaded or peptide-loaded autologous DC. After 7 days of culture, CD8⁺ T cells were stained with tetramers and analyzed by flow cytometry. Peptide-loaded. DC, but not unloaded DC, induced massive expansion, of epitope-specific CD8⁺ T cells, as shown by the high frequency of tetramer positive cells at day 7, and decreased PKH fluorescence intensity of tetramer positive cells (FIG. 6A). However, the response of tetramer positive cells to the peptide stimulation was heterogeneous: part of the cells did not divide. We noticed that a fraction of tetramer negative cells also divided but in similar proportion in cultures with peptide-loaded and unloaded DC, indicating that this proliferation was not specific for the peptide.

In order to quantify the proportion of precursor T cells that have divided and the number of cell divisions, we performed deconvolution analysis using the ModFit software (FIG. 6B). CD8 T cells stimulated with peptide-loaded DC have undergone up to 9 divisions. This approach enabled us to draw, on one hand the distribution of day 7 tetramer positive cells among generations, and on the other hand, the day 7 distribution of day 0 starting cells, in order to follow the fate of precursors. To rule out any donor specific behavior, two epitope-specific CD8 T cell populations were analyzed for each volunteer. As shown in FIG. 7A, the distribution of effector-memory T lymphocytes CD8 cells specific for EBV1 and central-memory T lymphocytes specific for EBV2 was similar. The majority of cells stained by tetramers at day 7 have undergone 5 to 6 divisions. For both epitopes however, these cells are the progeny of only 25% of ex vivo tetramer positive cells (15 and 10% have divided 5 or 6 times, respectively), the majority of these precursors dividing less than twice (FIG. 7B). For the different donors tested, the proliferation potential could be described by two parameters: the mean division of dividing precursors (PMD) and the percentage of precursors dividing at least twice (PF). Data obtained for EBV1, EBV2 and CMV1 specific CD8 T cells are summarized in FIGS. 7C and D. PMD was relatively constant among volunteers and among the different CD8 T cell subsets, with a mean division number of 4 to 5 (FIG. 7C). The recruitment was more heterogeneous, varying from 20% to 70% of initial specific CD8⁺ cells (FIG. 7D). Despite this heterogeneity, the three CD8 T cell subsets contained similar proportion of precursors able to proliferate; most variations were found among donors, not among epitopes. All together, these results show that the three epitope-specific CD8 populations display similar capacity to be recruited and proliferate after antigenic stimulation in vitro.

### EXAMPLE 4

Dendritic cell (DC) maturation is triggered in peripheral tissues by pathogen-derived or pro-inflammatory signals: it entrains enhanced Ag presentation and costimulation by DC, concomitant to migration to draining lymphoid organs for naïve T cell priming (Banchereau & Steinman, Nature 1998. 392: 245-252). The influence of DC maturation on T cell recruitment, activation, expansion and functional differentiation is currently widely investigated. Downstream events of the DC activation process are influenced by multiple variables: on one hand, the nature of the activating stimuli and the modulating influence of environmental factors (Vieira et al., J Immunol 2000. 164: 4507-4512), on the other, kinetics of DC activation and cytokine release (Langenkamp et al., Nature Immunol 2000. 1: 311-316; Kalinski et al., J Immunol 1999. 162: 3231-3236). The nature and relevance of help for CTL priming represent an additional issue. CD4⁺ T cells “license.” DC for CD8⁺ T cell activation or directly affect CD8+ T cell (Ridge et al., Nature 1998. 393: 474-478; Lu et al., J Exp Med 2000. 191: 541-550). CD4-independent CTL responses were also reported, particularly during viral infections (Buller et al., Nature 1987. 328: 77-79), as well as in conditions where Ag was not limiting and/or specific CD8⁺ T cell precursor frequency was elevated (Wang et al., J Immunol 2001. 167: 1283-1289; Mintern et al., J Immunol 2002. 168: 977-980). However, requirements for DC to be able to prime CD8⁺ T cell effector functions in absence of CD4 help have not been fully clarified.

Physiologically, DC maturation is triggered simultaneously by several active species, rather than by a single defined agent. Therefore, we focused our study on a previously identified bacterial extract (Boccaccio et al., J Immunotherapy 2002. 25: 88-96), comparing it to a cocktail of polyI:C (synthetic double-stranded RNA) plus an agonist anti-CD40 mAb. We first sought to determine if maturation time and agent affect the priming abilities of DC, next to characterize the Ag-specific CD8⁺ T cells expanded in terms of frequency, effector function and precursor recruitment. To this end, we used a model of in vitro priming of CD8⁺ T cells specific for the immunodominant epitope of the melanoma-associated Ag Melan-AMART1 (Melan-A) (Coulie et al., J Exp Med 1994. 180: 35-42; Kawakami et al., J Exp Med 1994. 180: 347-352). In healthy individuals, naive Melan-A-specific CD8⁺ T cells are present at relatively high frequencies in both adult and cord blood, as a consequence of a remarkably efficient thymic selection (Pittet et al., J Exp Med 1999. 190: 705-715; Zippelius et al., J Exp Med 2002, 195: 485-494). Therefore, the Melan-A epitope represents a unique model antigen to quantitatively study Ag-specific T cell priming in human. We found that DC ability to both recruit and expand a broad repertoire of Ag-specific CD8⁺ T cells is strongly influenced by their stage of maturation: in absence of exogenous cytokines and CD4 help, only DC engaged in-the maturation process and actively secreting IL-12 were effective in inducing CTL responses. In spite of this, the Ag-specific T cells expanded presented similar overall avidity.

### MATERIALS AND METHODS

Peptides and Tetramers.

Melan-A_{26-35(27L)} (ELAGIGILTV, Valmori et al., 1998) and PSA1₁₄₁₋₁₅₀ (FLTPKKLQCV) peptides were from Neosystem (Strasbourg, France). PE-labelled HLA-A*0201 tetramers contained the following peptides: Melan-A_{26-35 (27L)}, EBV BMFL-I₂₈₀₋₂₈₈ (GLCTLVAML), PSA3₁₅₄₋₁₆₃ (VLSNDVCAQV) (Proimmune, Oxford, UK) and influenza matrix protein M1₅₈₋₆₆ (GILGFVFTL) (Beckman Coulter Immunomics, San Diego, Calif.).

DC Differentiation and Maturation.

DC differentiation was performed with VacCell processor (IDM, Paris, France) as previously described (Goxe et al., Immunol Invest 2000. 29: 319-336; Boccaccio et al., J Immunotherapy 2002. 25: 88-96). Briefly, PBMC were cultured for 7 days in serum-free IDM VacCell medium (Life Technologies, Paisley, UK) supplemented with 500 U/ml GM-CSF (Novartis Pharma AG, Basel, Switzerland) and 50 ng/ml IL-13 (Sanofi-Synthelabo, Paris, France). DC were then isolated by elutriation. Purity ranged from 80 to 99%; viability was >95%. In some instances, DC were frozen in a solution of 4% human albumin containing 10% DMSO, then maturated after thawing and overnight recovery. For maturation, 2×10⁶ DC/ml were cultured in 24-well plates for 3 to 40 h in presence of various combinations of the following reagents: 1 μg/ml bacterial extract (Ribomunyl, Pierre Fabre Medicament, Boulogne, France), 500 U/ml IFN-γ (Imukin, Boehringer Ingelheim, Paris, France), 100 μg/ml polyriboinosinic-polyribocytidylic acid (polyI:C, Sigma), 2 μg/ml anti-CD40 mAb (mouse IgG1, clone J285, gift of Y. Richard, INSERM U131, Clamart, France). For kinetics experiments, 3, 6, or 20 h after addition of maturation agents, supernatants were collected for cytokine analysis, then DC were either harvested and used to stimulate CD8⁺ T cells, or gently washed and further cultured until the 40 h time point in the absence of maturation agents. We verified that after peptide pulsing and mitomycin C treatment (same protocol as for T cell stimulation, see below), DC were still undergoing maturation and secreting levels of IL-12 p70 and IL-10 comparable to untreated DC.

Elisa.

IL-12 p70, IL-10, TNF-α, IL-6, IL-1β, IL-15, IL-2, TGF-β, IL-4, and IL-7 were measured by ELISA using antibody pairs from R&D Systems. Europe (Abingdon, UK) according to manufacturer's instructions.

CD8⁺ T Cells Isolation and Stimulation.

CD8⁺ T cells were purified by negative selection using CD8⁺ T cell Isolation Kit (Miltenyi Biotec, Paris, France). Among viable cells, CD³⁺/CD8⁺/CD4⁻ cells were 86±4%, CD3⁺/CD4⁺/CD8⁻ cells 0.1±0.2%, CD56⁺/CD3⁻ cells 0.1±0.1%. Non-matured or matured DC were pulsed for 2 h at 37° C. with 10 μg/ml Melan-A peptide and 5 μg/ml β2-microglobulin, treated with mitomycin C, and extensively washed. CD8⁺ T cells (1.5×10⁵/well) were cocultured with peptide-pulsed autologous DC (3×10⁴/well) in 96-well U-bottom plates in Iscove's medium (supplemented with 10% autologous serum, L-arginine, L-asparagine and L-glutamine) in the presence or absence of 1000 U/ml IL-6 and 5 ng/ml IL-12. For IL-12 blocking experiments, 15 μg/ml anti-human IL-12 mAb (clone 24910.1, R&D Systems) and/or 10 μg/ml of anti-IL-12R mAbs clones 2.4E6 and 2B10 (BD Pharmingen), or isotype controls were added to microcultures. On day 7 and 14, DC were thawed, matured, pulsed with Melan-A peptide and used to restimulate the T cells, in the presence or absence of 20 U/ml IL-2 and 10 ng/ml IL-7. Eight T cell microcultures were stimulated for each DC condition and independently tested.

Cytotoxicity Assay.

T cell microcultures were assessed on day 14 or 21 in a standard 4-h ⁵¹Cr-release assay for their capacity to lyse TAP-deficient T2 cells in presence of 1 μM Melan-A or PSA1 peptide, 0.5 μg/ml β₂-microglobulin and K562 cells. For the avidity assay, graded concentrations of Melan-A peptide (from 0.1 pM to 1 μM final) were added to T2 cells before addition of effectors.

IFN-γ Elispot Assay.

T2 cells were pulsed for 1 h at 37° C. with Melan-A or PSA1 peptide (10 μg/ml) in the presence of 5 μg/ml β₂-microglobulin. T cells (300/well) and T2 cells (5×10⁴/well) resuspended in complete Iscove's medium were then seeded in Multiscreen nitrocellulose 96-well plates (Millipore, Bedford, Mass.) precoated with anti-IFN-γ mAb (1-D1K, Mabtech, Stockholm, Sweden). Individual T cells microcultures were tested in duplicate. Controls included T cells and T2 cells alone, or T cells in presence of T2 cells and 10 μg/ml PHA. After 20 h incubation at 37° C., plates were washed, incubated with biotinylated anti-IFN-γ mAb (7-B6-1, Mabtech) and stained with Vectastain Elite kit (Vector Laboratories, Burlingame, Calif.). Spot Forming Cells (SFC) were counted with a stereomicroscope (Carl Zeiss, Le Pecq, France).

CD8⁺ T Cell Tetramer Staining and Immunophenotype

T cells were incubated with A2/tetramers, then with FITC, PerCP, or APC-conjugated anti-CD45RA, anti-CD8, anti-CD3 mAb or isotype controls (Immunotech, Marseille, France). For CCR7 staining, anti-hCCR7 mAb (clone CCR7.6B3, eBioscience, San Diego, Calif.) was added together with the A2/tetramers, then cells were incubated with FITC-labeled goat Abs anti-mouse IgG1 (Southern Biotechnology Associates, Birmingham, USA), washed, and stained with anti-CD8 and anti-CD45RA. For flow cytometry analysis, cells were resuspended in PBS containing 3 nM TO-PRO-3 (Molecular Probes, Leiden, UK) or 1 μg/ml propidium iodide as dead-exclusion dyes. At least 100,000 viable events were acquired on a FACSCalibur with CellQuestPro software (Becton Dickinson, St. Jose, Calif.).

PKH-Dilution Assay.

Purified CD8⁺ cells were labeled with PKH67 (Sigma, St. Louis, Mo.) according to manufacturer instructions. Labeled cells were stimulated once in absence of exogenous cytokines with Melan-A or PSA1-pulsed, matured or non-matured DC. IL-12 blocking experiments were performed as described above. On day 8, CD8⁺ cells from 4 microcultures of the same condition of stimulation were pooled, washed, stained with A2/tetramers, anti-CD3 or anti-CD8 mAb, dead-exclusion dye, and analyzed by flow cytometry. The precursor frequencies (PF) and proliferation indexes (PI) among gated viable CD8⁺/Melan-A tetramer⁺ cells were calculated as described previously (Givan et al., J Immunol Methods, 1999. 230: 99-112) with ModFit software (Verity Software House, Topsham, Me.).

### RESULTS

DC Maturation: Kinetics of Cytokine Secretion.

The influence of maturation time on DC surface marker expression and cytokine secretion was first defined. DC were exposed to maturation agents for 3, 6, 20, or 40 h, washed, then further cultured until the 40 h time point. Cytokines known to be relevant for T cell activation were measured in supernatants (Geginat et al., J Exp Med 2001. 194: 1711-9). A contact as short as 3 h with the bacterial extract could trigger DC maturation, driving the process of up-regulation of CD80, CD86, CD40, MHC class I, CD83 and CD25 to completion within 20 h (Boccaccio et al., J Immunotherapy 2002. 25: 88-96, and not shown). This brief exposure to maturation agent was also sufficient to induce a significant secretion of IL-12 p70, which occurred from 6 to 20 h from the initiation of maturation (FIG. 8). Untreated DC secreted baseline levels of IL-10, but upon maturation a peak of production was also observed during the interval from 6 to 20 h. TNF-α was released earlier, generally within the first 6 h of maturation, similar to IL-6. Low but significant levels of IL-1β and IL-15, mostly produced after 6 h of maturation were found. The association of IFN-γ to the maturation agent drastically increased the amounts of IL-12 p70 produced without altering the kinetics of release. In some cases and to a lesser extent, TNF-α production was also enhanced by addition of IFN-γ during maturation, whereas no significant modulation was seen on IL-6, IL-1β and IL-15 secretion. By contrast, a 6-h contact with polyI:C and anti-CD40 mAb was not sufficient for triggering complete DC maturation, as assessed by surface marker expression (Boccaccio et al., J Immunotherapy 2002. 25: 88-96) and cytokine secretion (FIG. 8). With the exception of IL-1β and IL-15, DC activated with polyI:C/anti-CD40 produced lower levels of cytokines compared to activation with the bacterial extract, but kinetics of release were similar. We could not detect IL-2, TGF-β, IL-4 or IL-7 in DC supernatants.

Thus, following 20 h of maturation, DC secrete very low amounts of IL-12 p70, IL-10, TNF-α, and IL-6, in contrast to “maturing” DC generated by a short exposure to the bacterial extract and IFN-γ. We therefore examined the influence of time of maturation on DC ability to prime Ag-specific CD8⁺ T cells.

DC Maturation Time Influences Melan-A-Specific CD8⁺ T Cell Induction.

Untreated DC or DC exposed to bacterial extract+IFN-γ for 3, 6, or 20 h were pulsed with the analogue Melan-A_{26-35(27L)} peptide and used to stimulate autologous CD8⁺ T cells in absence of exogenous cytokines or growth factors. Alternatively, maturation agents were added in T cell microcultures together with Melan-A-pulsed DC: in this case, maturation occurred concomitantly to T cell priming. After 2 stimulations, CD8⁺ T cells were tested by IFN-γ-ELISPOT and in ⁵¹Cr-release assay. As shown in FIG. 9A, “maturing” DC (that is, DC activated either by a short contact with maturation agent or concomitant to T cell priming) were quite efficient in the generation of Melan-A-specific CD8⁺ T cells: based on estimation by ELISPOT, their frequencies ranged from 50 to 100% of T cells, depending on the condition of stimulation. Six hours of maturation consistently allowed to obtain frequencies of Melan-A specific T cells similar to those obtained when maturation agents were added to cocultures (i.e., independent of maturation time). These T cells also demonstrated specific cytotoxicity against T2 target cells loaded with Melan-A peptide (FIG. 9B). On the contrary, CTL could not be efficiently induced by non-matured DC or DC exposed for 20 h to the bacterial extract, indicating that the stage of DC maturation is a critical parameter for the generation of type-1 effector CD8⁺ T cells in the absence of CD4 help.

DC Maturation Agents Influence Melan-A-Specific CD8⁺ T Cell Induction.

Given the strong modulating effect of IFN-γ on IL-12 p70 and IL-10 secretion by DC, we evaluated the priming abilities of DC matured in the absence of IFN-γ. We also extended this analysis to a different cocktail of maturation agents, polyI:C and anti-CD40 mAb.

DC activated for 6 h induced important frequencies of Melan-A-specific T cell, but to a different extent depending on the maturation agent: 30, 47, and 91% following DC treatment with polyI:C/anti-CD40, bacterial extract, and bacterial extract+IFN-γ, respectively (FIG. 10A). T cell microcultures were split during in vitro stimulations (except when stimulated with non-matured DC), and overall T cell proliferation was also at least twice more important upon stimulation with DC matured in the presence of IFN-γ compared to the other stimuli. Taking into account both total T cell number and frequency of effector T cells detected by ELISPOT, we calculated that DC matured for 6 h in the presence of IFN-γ could generate up to 2000-fold more Ag-specific T cells than non-matured DC (compared to approximately 500-fold for bacterial extract alone or polyI:C/anti-CD40). Regardless of the maturation factor(s) used, 20 h-matured DC were inefficient inducers of Melan-A-specific IFN-γ-secreting T cells (FIG. 10B). However, high T cells frequencies could be obtained when maturation agents were directly added to the Melan-A-loaded DC and T cells cocultures. Cytotoxicity data were concordant with ELISPOT (not shown).

Finally, when exogenous cytokines were added during T cell induction (IL-12 and IL-6 during the first stimulation, IL-2 and IL-7 during the subsequent ones), Melan-A-specific CTL were generated not only by 20 h-matured DC but even in the absence of maturation agents (FIG. 10C).

Influence of DC Maturation Agents on Melan-A-Specific CD8⁺ T Cell Functional Differentiation.

As conditions of DC maturation affect the frequency and number of Melan-A-specific CD8⁺ cells, T cell function and phenotype were evaluated. We found similar overall avidity of the CTL populations obtained after 2 stimulations with DC activated for 6 h with polyI:C/anti-CD40, bacterial extract, or bacterial extract+IFN-γ (FIG. 11). Frequencies of Melan-A-specific CD8⁺ T cells in these populations were respectively 1.5%, 16% and 51% based on tetramer staining (see also FIG. 12 below). Thus, the maximal lytic activity obtained by varying the concentrations of Melan-A peptide on target cells was dependent on the condition of stimulation (6% for non-matured DC, 8.5% for polyI:C/anti-CD40, 37% for bacterial extract, and 47% for bacterial extract+IFN-γ). However, 50% maximal lysis required in all cases 100 pM of peptide (FIG. 11).

One also analyzed the expression of CCR7 and CD45RA in the Melan-A-specific CD8⁺ T cell populations identified by tetramer staining. Melan-A specific CD8⁺ T cells expressed both CD45RA and CCR7 prior to stimulation with DC (FIG. 12), consistent with a naive phenotype (Pittet et al., J Exp Med 1999. 190: 705-715; Zippelius et al., J Exp Med 2002. 195: 485-494). After stimulation with non-matured DC in the absence of exogenous cytokines, most of specific CD8⁺ cells (63%) conserved the same surface marker expression, and only a minority appeared to progressively down-regulate CD45RA expression. Both CD45RA and CCR7 were lost from the majority of Melan-A specific T cells upon expansion with maturing DC, in agreement with differentiation of effector cells. However, 10 to 40% of the Melan-A-specific CD8⁺ T cells that were primed with polyI:C/anti-CD40-treated DC still maintained CCR7 expression. We found no expansion of CD4⁺/CD8⁻ T cells after 2 stimulations with DC (0.05±0.01% of viable CD3+ T cells), indicating that CD8⁺ priming occurred in the absence of CD4 help.

Taken together, these results strongly suggest that the levels of cytotoxicity obtained in the various conditions of CD8⁺ T cells stimulation are mostly a consequence of final CTL frequency rather than of different functional avidity or effector state of the expanded repertoire.

Analysis of T Cell Recruitment and Proliferation.

The next objective was to analyze T cell proliferation and precursor recruitment after priming with DC at various stages of maturation. The proliferation of Melan-A-specific T cell was evaluated by associating the PKH-dilution assay (Givan et al., J Immunol Methods, 1999. 230: 99-112) with tetramer staining (Bercovici et al., J Immunol Methods. 2003. 276 (1-2):5-17). Purified CD8⁺ T cells were labeled with PKH67, then stimulated in vitro, in the absence of exogenous cytokines, with Melan-A or control peptide-pulsed DC that were either left untreated, or exposed to the bacterial extract and IFN-γ for 3, 6, 20 h. Alternatively, maturation agents were added together with peptide-pulsed DC and CD8⁺ T cells (maturation during priming). After 8 days of culture, CD8⁺ T cells were stained with A2/Melan-A tetramers and analyzed by flow cytometry. As shown in FIG. 13A, Melan-A-specific T cells underwent a strong proliferation if stimulated by DC pulsed with specific peptide but not with control peptide. In addition, staining with control tetramers did not show expansion of influenza matrix protein or EBV-specific memory CD8⁺ cells (FIG. 13B), further indicating that the Melan-A-specific T cell recruitment was strictly Ag-dependent. Frequencies of specific CD8⁺ cells after one in vitro stimulation were in this experiment 4.7, 4.9, and 0.9% for 3-h, 6-h, and 20-h activated DC respectively. Maximal expansion of Melan-A-specific T cells was obtained when DC maturation occurred concomitant to priming (7.1% of CD8⁺ T cells).

A proliferation of Melan-A tetramer negative CD8⁺ cells was also induced (FIG. 13), which expanded proportionally to Melan-A specific CD8^{+cells. However, it was not a population of Melan-A-specific T cells, as it was also induced by PSA}1-pulsed DC (FIG. 13A) and in HLA-A2 negative donors (not shown). When CD8⁺ cells where stimulated in the presence of both anti-IL-12 p70 and anti-IL-12Rβ1 mAbs, maturing DC-induced proliferation of Melan-A-positive cells was reduced to the levels obtained with non-matured DC (FIG. 13C), and IFN-γ secretion 95% blocked (not shown). The anti-IL-12 or the anti-IL-12R mAbs used separately could partially inhibit T cell priming by DC activated in the absence of IFN-γ (not shown).

We then determined the precursor frequencies (PF) and the proliferation indexes (PI) of specific T cells mobilized in the different conditions of stimulation (Givan et al., J Immunol Methods, 1999. 230: 99-112). PF indicates, among total Melan-A precursors, the fraction of Melan-A-specific T cells proliferating after stimulation. DC maturation time affected both the number of precursors recruited and their intensity of proliferation: compared to 20 h and non-matured DC, 6 h-activated DC mobilized a 2 to 30-fold higher fraction of Melan-A precursors and sustained a 2 to 24-fold higher specific T cell proliferation (Table III). For 6 h-activated DC, best T cell mobilization was obtained by associating IFN-γ to the bacterial extract (Table IV). In this case, DC recruited 2.5 and 4 times more precursors (which proliferated on average 3 and 6-fold more) than when maturated with the bacterial extract alone or polyI:C/anti-CD40, respectively. Maturation time was a critical limiting parameter for polyI:C/anti-CD40-treated DC, because precursor recruitment was distinctly higher when maturation was concomitant to T cell priming (not shown).

Therefore, the high frequencies of Melan-A-specific effector CD8⁺ T cells expanded by DC activated with a bacterial extract in presence of IFN-γ were consequent to both important recruitment and intense proliferation of naive Melan-A precursors and dependent on IL-12 secretion by DC.

### DISCUSSION

In this study, we found that both matured and non-matured DC have the potential to prime naïve T cells in vitro, but that only “maturing”, IL-12-secreting DC efficiently do so in absence of exogenous cytokines. Maturation stage regulates DC capacity to both recruit and sustain proliferation of naïve T cells, leading to different final CTL frequencies.

Priming and Functional Differentiation of Ag-Specific CD8⁺ Cells.

In healthy donors, the Melan-A-specific CD8⁺ precursors display a naive phenotype (FIG. 11 and Pittet et al., J Exp Med 1999. 190: 705-715; Zippelius et al., J Exp Med 2002. 195: 485-494). Analysis of surface marker expression suggested their functional maturation towards an effector memory phenotype (Sallusto et al., Nature 1999. 401: 708-712) upon stimulation with activated DC (FIG. 11). However, a population of Melan-A-specific T cells with heterogenous phenotype (CD45RA⁻, CCR7⁺ and ⁻ cells) was generated in the presence of polyI:C/anti-CD40-treated DC. Whether this is related to an incomplete polarization or to the generation of 2 independent subsets of memory/effector T cells remains to be tested.

The levels of cytotoxicity of individual T cell microcultures directly correlated with their frequency in IFN-γ-secreting, Melan-A-specific T cells. Therefore, although IFN-γ-secreting and cytolytic CD8⁺ cells may represent two distinct subsets (Sandberg et al., J Immunol 2001. 167: 181-187), both were preferentially expanded by the same DC maturation conditions. CD8⁺ cells expanded by DC activated with the various maturation agents showed comparable avidity, indicating that the differences in cytotoxicity levels observed were mostly a consequence of specific T cells frequencies.

Mature and Immature CD: Requirements for Priming.

It was previously shown that mature DC are required for optimal generation of memory CD8⁺ T cells, both in vitro (Larsson et al., 2000) and in vivo (Jonuleit et al., Int J Cancer 2001. 93: 243-251; Dhodapkar et al., J Exp Med 2001. 193: 233-238). Lapointe and associates efficiently generated IFN-γ-secreting, Melan-A-specific T cells upon addition of multiple activation signals to DC (Lapointe et al., Eur J Immunol 2000. 30: 3291-3298). Zarling et al. described that both immature and mature DC were able to induce primary HIV-specific CTL responses in vitro (Zarling et al., J Immunol 1999. 162: 5197-5204). However, IL-2 and IL-7 were added during T cell stimulation. Secondly, differences between the two DC populations may have been blunted by the fact that DC were matured for at least 3 days, and final DC maturation results in impaired ability to produce several cytokines, including IL-12 (Langenkamp et al., Nature Immunol 2000. 1: 311-316; Kalinski et al., J Immunol 1999. 162: 3231-3236, and FIG. 8). This inefficiency of terminally mature DC in secreting IL-12 and priming naive T cells may be critical for prevention of infection-induced immunopathology (Reis e Sousa et al., Immunity 1999. 11: 637-647). We therefore deem important to consider the priming potential acquired by DC along the dynamic process of maturation, besides strictly comparing mature versus immature DC.

Maturation Agents and Cytokine Secretion: IL-12 p70.

Despite strong up-regulation of costimulatory and MHC molecules, DC maturated with polyI:C/anti-CD40 demonstrated low secretion of the cytokines tested, with the exception of IL-1β and IL-15 (FIG. 8). Moreover, we previously showed that 6 h of contact with polyI:C/anti-CD40 were not sufficient for commitment to full maturation (Boccaccio et al., J Immunotherapy 2002. 25: 88-96). Taken together, these results can explain the lower priming ability of DC activated for 6 h with this cocktail compared to the bacterial extract.

The generation of high frequencies of CTL by non-matured DC upon addition of exogenous cytokines, the sub-optimal stimulation by DC activated for 20 h and the enhanced priming abilities of DC maturated in the presence of IFN-γ, all suggest a critical role for cytokine produced by maturing DC during the in vitro induction of Melan-A-specific effector CD8⁺ cells. IFN-γ is not by itself a DC maturation factor. Yet, its presence during maturation significantly increased both recruitment and proliferation of Melan-A-specific precursors (Table IV), concomitant with enhanced secretion of IL-12 but not of other cytokines. Indeed, blockade of both IL-12 and IL-12β1 led to strong inhibition of maturing DC-driven T cell proliferation and effector function (FIG. 13C and not shown). As the anti-IL-12Rβ1 mAbs used may also neutralize IL-23 activity, a role for this cytokine cannot be excluded. In p40−/− mice, IL-12 is not required for CTL priming (Wan et al., J Immunol 2001. 167: 5027-5033), suggesting that additional mechanisms may be important in vivo, but not excluding a critical influence of IL-12 on the outcome of the immune response. A refined analysis of the importance of soluble factors versus contact-mediated signals in T cell priming will determine if cytokines are specifically important for T cells survival and expansion, or also crucial during the initial step of T cell activation and/or functional differentiation.

In vivo, “maturation-triggered” monocyte-derived DC should rapidly migrate to draining lymph node concomitant to up-regulation of CCR7 expression and responsiveness to MIP-3β (Dieu et al., J Exp Med 1998. 188:373-386). However, the in vivo localization of maturing DC during the 12 h of their enhanced IL-12 secretion remains to be studied, and will likely be different depending on DC subsets and mode of activation.

Helper Dependence for CD8⁺ T Cells Priming.

Although it was not the aim of this work to dissect the mechanism of help for CTL priming, we observed that the presence of CD4⁺ T cells was not a requirement for inducing Melan-A-specific CTL when using maturing DC as stimulators. DC activated with the bacterial extract were at least as good in priming as DC exposed to the agonist anti-CD40 mAb. In the purified CD8⁺ T cell samples used in this study, contamination with CD4⁺ T cells was always less than 0.8%. Despite the fact that no nominal class II Ag was added, and autologous sera were used throughout the study (ruling out possible responses against FCS-derived proteins), MHC class II epitopes might have been provided by the bacterial extract. Nonetheless, we propose that soluble factors secreted by DC during particular stages of maturation might be sufficient to sustain CTL activation and proliferation. In addition, help may have been provided by the CD8⁺ cells themselves via either autocrine cytokine production or feed-back DC-activation (Wang et al., J Immunol 2001. 167: 1283-1289; Mintern et al., J Immunol 2002. 168: 977-980; Mailliard et al., J Exp Med 2002. 195: 473-483). Because of the singularly high frequency of Melan-A precursors, additional experimental validation is required to generalize these findings to other naive, Ag-specific, low frequency T cells. Proliferation of CD8⁺ cells with undefined specificities observed upon stimulation with maturing DC (FIG. 13) may also have contributed to Melan-A-specific T cell expansion. As such proliferation was also seen with polyI:C/anti-CD40-activated DC (preferentially in the condition of maturation during in vitro stimulation), it is unlikely that it simply represent a population specific for Ag present in the bacterial extract. Maturing DC secrete several cytokines that were reported to drive Ag-independent proliferation of memory and effector T cells, including IL-15 (Geginat et al., J Exp Med 2001. 194: 1711-9): yet, in conditions of maximal proliferation of these CD8⁺ cells with undefined specificities, we could not detect expansion of T cells belonging to the memory pool, as influenza or EBV-specific T cells (FIG. 13B).

Taken together, these results suggest that stages of DC maturation, affecting both recruitment and proliferation of naive CD8⁺ T cells, are of crucial relevance for the induction of primary CTL responses and might influence requirement for CD4 help.

### EXAMPLE 5

The dendritic cells are prepared according to WO 03/010301. 3×10⁴ HLA-A2 positive human DC matured 6 hours with FMKp and IFNγ are loaded with Melan-A _{26-35 (27L)} peptide in concentration ranging from 0.001 ng/ml to 100 μg/ml.

3×10³ Melan-A specific T cell clone displaying a T cell receptor specific for Melan A are stained with the PKH67 fluorescent dye at 2 μM to track cell proliferation in response to the specific antigen loaded into dendritic cells.

The labeled T cells are stimulated for at least 6 days by the dendritic cells loaded with the different concentration of the antigen specific to the T cell receptor (Melan-A _{26-35 (27L)}) in the presence of IL-2 and supernatant of MLA cell line.

After 6 days, T cells are labeled with anti-CD8 antibody and tetramer specific for Melan-A_{26-35 (27L)} peptide. Analysis is performed by flow cytometry as described previously. The precursor frequencies and proliferation indices were calculated with modFit software as described previously. The proliferative responses obtained according to the different concentrations of antigen tested are used to establish a dose response curve which may be used as a standard T-cell control response of T lymphocytes.

The FIG. 15 represents one point of such curve (upper row). The PSA1 is an irrelevant antigen used as negative control.

The precursor frequencies and proliferation indices were calculated with modFit software. Melan-A_{26-35 (27L)} upper panel The calculated Proliferation Index of the T-cells contacted with dendritic cells loaded with Melan-A_{26-35 (27L)} is 3.54 and the Precursor Frequency is 86.5%. The calculated Proliferation Index of the T-cells contacted with dendritic cells loaded with PSA1 is 1.12 and the Precursor Frequency is 3.4%.

One skilled in the art readily appreciates that the present invention is well adapted to carry out the objectives and obtain the ends and advantages mentioned as well as those inherent therein. Methods, applications and uses described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### BIBLIOGRAPHY

Allsopp C E, Nicholls S J and Langhorne J, A flow cytometry method to assess antigen-specific proliferative responses of different subpopulations of fresh and cryopreserved human peripheral blood mononuclear cells. J Immunol Methods 1998. 214: 175-186.
Altman J D, Moss P A, Goulder P J, Barouch D H, McHeyzer-Williams M G, Bell J L, McMichael A J and Davis M M, Phenotypic analysis of antigen-specific T lymphocytes. Science 1996. 274: 94-96.
Appay V, and Rowland-Jones S, The assessment of antigen-specific CD8⁺ T cells through the combination of MHC class I tetramer and intracellular staining. J Immunol Methods 2002. 268: 9.
Ashley D M, Faiola B, Nair S, Hale L P, Bigner D D, Gilboa E., Bone marrow-generated dendritic cells pulsed with tumor extracts or tumor RNA induce antitumor immunity against central nervous system tumors, J Exp Med. 1997, 186: 1177-82.
Banchereau J and Steinman R M, Dendritic cells and the control of immunity. Nature 1998. 392: 245-252.
Bercovici N, Duffour M T, Agrawal S, Salcedo M and Abastado J P, New methods for assessing T-cell responses. Clin Diagn Lab Immunol 2000. 7, 859-64.
Bercovici N, Givan A L, Waugh M G, Fisher J L, Vernal-Pauillac F, Ernstoff MS, Abastado J P, Wallace P K., Multiparameter precursor analysis of T-cell responses to antigen. J Immunol Methods. 2003. 276 (1-2):5-17.
Bird J J, Brown D R, Mullen A C, Moskowitz N H, Mahowald M A, Sider J R, Gajewski T F, Wang C R and Reiner S L, Helper T cell differentiation is controlled by the cell cycle Immunity 1998. 9: 229-237.
Boccaccio C, Jacod S, Kaiser A, Boyer A, Abastado J P and Nardin A, Identification of a clinical-grade maturation factor for dendritic cells. J Immunotherapy 2002. 25: 88-96.
Bubenik J. Genetically engineered dendritic cell-based cancer vaccines. Int J Oncol. 2001 18: 475-8.
Buller R M, Holmes K L, Hugin A, Frederickson T N and Morse H C, 3rd, Induction of cytotoxic T-cell responses in vivo in the absence of CD4 helper cells. Nature 1987. 328: 77-79.
Bungener L, Huckriede A, Wilschut J, Daemen T, Delivery of protein antigens to the immune system by fusion-active virosomes: a comparison with liposomes and ISCOMs. Biosci Rep. 200 22: 323-38.
Champagne P, Dumont A R and Sekaly R P, Learning to remember: generation and maintenance of T-cell memory. DNA Cell Biol 2001. 20: 745-760.
Champagne P, Ogg G S, King A S, Knabenhans C, Ellefsen K, Nobile M, Appay V, Rizzardi G P, Fleury S, Lipp M, Forster R, Rowland-Jones S, Sekaly R P, McMichael A J and Pantaleo G, Skewed maturation of memory HIV-specific CD8 T lymphocytes. Nature 2001. 410: 106-111.
Cohen C J, Denkberg G, Lev A, Epel M and Reiter Y, Recombinant antibodies with MHC-restricted, peptide-specific, T-cell receptor-like specificity: new tools to study antigen presentation and TCR-peptide-MHC interactions. J Mol Recognit. 2003. 16:324-32.
Coulie P G, Brichard V, Van Pel A, Wolfel T, Schneider J, Traversari C, Mattei S, De Plaen E, Lurquin C, Szikora J P and et al., A new gene coding for a differentiation antigen recognized by autologous cytolytic T lymphocytes on HLA-A2 melanomas. J Exp Med 1994. 180: 35-42.
Czerkinsky C C, Nilsson L A, Nygren H, Ouchterlony O and Tarkowslci A, A solid-phase enzyme-linked immunospot (ELISPOT) assay for enumeration of specific antibody-secreting cells. J Immunol Methods. 1983. 65 (1-2): 109-121.
Dhodapkar M V, Steinman R M, Krasovsky J, Munz C and Bhardwaj N, Antigen-specific inhibition of effector T cell function in humans after injection of immature dendritic cells. J Exp Med 2001. 193: 233-238.
Dieu M C, Vanberliet B, Vicari A, Bridon J M, Oldham E, Ait-Yahia S, Briere F, Zlotnik A, Lebecque S, and Caux C. Selective recruitment of immature and mature dendritic cells by distinct chemokines expressed in different anatomic sites. J Exp Med 1998. 188:373-386
Dutoit V, Rubio-Godoy V, Pittet M J, Zippelius A, Dietrich, P Y, Legal F A, Guillaume P. Romero P, Cerottini J C, Houghten R A, Pinilla C and Valmori D, Degeneracy of antigen recognition as the molecular basis for the high frequency of naive A2/Melan-a peptide multimer(+) CD8(+) T cells in humans. J Exp Med, 2002. 196, 207-16.
Fields R C, Shimizu K, Mule J J., Murine dendritic cells pulsed with whole tumor lysates mediate potent antitumor immune responses in vitro and in vivo, Proc Natl Acad Sci USA. 1998. 95: 9482-7.
Ge Q, Stone J D, Thompson T D, Cochran J R, Rushe M, Eisen H N, Chen J and Stern L J, Soluble peptide-MHC monomers cause activation of CD8⁺ T cells through transfer of the peptide to T cell MHC molecules, Proceed Natl Acad Sci USA. 2002, 99:13729-13734.
Geginat J, Sallusto F and Lanzavecchia A, Cytokine-driven proliferation and differentiation of human naive, central memory, and effector/memory CD4(+) T cells. J Exp Med 2001. 194: 1711-9.
Gett A V and Hodgkin P D, A cellular calculus for signal integration by T cells, Nat Immunol 2000. 1 (3):239-244.
Gett A V and Hodgkin P D, Cell division regulates the T cell cytokine repertoire, revealing a mechanism underlying immune class regulation. Proc Natl Acad Sci USA 1998. 95: 9488-9493.
Givan A L, Fisher J L, Waugh M, Ernstoff M S and Wallace P K, A flow cytometric method to estimate the precursor frequencies of cells proliferating in response to specific antigens. J Immunol Methods, 1999. 230: 99-112.
Gottfried E, Krieg R, Eichelberg C, Andreesen R, Mackensen A, Krause S W, Characterization of cells prepared by dendritic cell-tumor cell fusion. Cancer Immun. 2002, 2: 15.
Goxe B, Latour N, Chokri M, Abastado J P and Salcedo M, Simplified method to generate large quantities of dendritic cells suitable for clinical applications. Immunol Invest 2000. 29: 319-336.
Gudmundsdottir H, Wells A D and Turka L A, Dynamics and requirements of T cell clonal expansion in vivo at the single-cell level: effector function is linked to proliferative capacity J Immunol 1999. 162: 5212-5223.
Herr W, Schneider J, Lohse A W, Meyer zum Buschenfelde K H and Wolfel T, Detection and quantification of blood-derived CD8+ T lymphocytes secreting tumor necrosis factor alpha in response to HLA-A2.1-binding melanoma and viral peptide antigens. J Immunol Methods 1996. 191: 131-142.
Horan P K and Slezak S E, Stable cell membrane labelling. Nature 1989. 340: 167-168.
Hosken N A, Bevan M J, Carbone F R, Class I-restricted presentation occurs without internalization or processing of exogenous antigenic peptides. J Immunol. 1989. 142:1079-83.
Jonuleit H, Giesecke-Tuettenberg A, Tuting T, Thurner-Schuler B, Stuge T B, Paragnik L, Kandemir A, Lee P P, Schuler G, Knop J and Enk A H, A comparison of two types of dendritic cell as adjuvants for the inductions of melanoma-specific T-cell responses in following intranodal injection. Int J Cancer 2001. 93: 243-251.
Jung T, Schauer U, Heusser C, Neumann C and Rieger C, Detection of intracellular cytokines by flow cytometry. J Immunol Methods 1993. 159: 197-207.
Kaech S M and Ahmed R, Memory CD8+ T cell differentiation: initial antigen encounter triggers a developmental program in naive cells. Nat Immunol 2001. 2: 415-422.
Kaech S M, Wherry E J and Ahmed R, Effector and memory T-cell differentiation: implications for vaccine development. Nat Rev Immunol 2002. 2: 251-262.
Kalinski P, Schuitemaker J H, Hilkens C M, Wierenga E A and Kapsenberg M L, Final maturation of dendritic cells is associated with impaired responsiveness to IFN-gamma and to bacterial IL-12 inducers: decreased ability of mature dendritic cells to produce IL-12 during the interaction with Th cells. J Immunol 1999. 162: 3231-3236.
Kassiotis G, Garcia S, Simpson E and Stockinger B, Impairment of immunological memory in the absence of MHC despite survival of memory T cells Nat Immunol 2002. 3: 244-250.
Kawakami Y, Eliyahu S, Sakaguchi K, Robbins P F, Rivoltini L, Yannelli J R, Appella E and Rosenberg S A, Identification of the immunodominant peptides of the MART-1 human melanoma antigen recognized by the majority of HLA-A2-restricted tumor infiltrating lymphocytes. J Exp Med 1994. 180: 347-352.
Klenerman P, Cerundolo V and Dunbar P R, Tracking T cells with tetramers: new tales from new tools. Nat Rev Immunol 2002. 2: 263-272.
Kotzin B L, Falta M T, Crawford F, Rosloniec E F, Bill J, Marrack P and Kappler J, Use of soluble peptide-DR4 tetramers to detect synovial T cells specific for cartilage antigens in patients with rheumatoid arthritis. Proceed Natl Acad Sci USA. 97:291-296.
Kugler A, Stuhler G, Walden P, Zoller G, Zobywalski A, Brossart P, Trefzer U, Ullrich S, Muller C A, Becker V, Gross A J, Hemmerlein B, Kanz L, Muller G A, Ringert R H, Regression of human metastatic renal cell carcinoma after vaccination with tumor cell-dendritic cell hybrids, Nat Med. 2000, 6: 332-6.
Langenkamp A, Messi M, Lanzavecchia A and Sallusto F, Kinetics of dendritic cell activation: impact on priming of TH1, TH2 and nonpolarized T cells. Nature Immunol 2000. 1: 311-316.
Lanzavecchia A and Sallusto F, Dynamics of T lymphocyte responses: intermediates, effectors, and memory cells. Science 2000. 290: 92-97.
Lapointe R, Toso J F, Butts C, Young H A and Hwu P, Human dendritic cells require multiple activation signals for the efficient generation of tumor antigen-specific T lymphocytes. Eur J Immunol 2000. 30: 3291-3298.
Larsson M, Messmer D, Somersan, S, Fonteneau J F, Donahoe S M, Lee, M, Dunbar P R, Cerundolo V, Julkunen I, Nixon D F and Bhardwaj N, Requirement of mature dendritic cells for efficient activation of influenza A-specific memory CD8+ T cells. J Immunol 2000. 165: 1182-1190.
Lee P P, Yee C, Savage P A, Fong L, Brookstedt D, Weber J S, Johnson D, Swetter S, Thompson J, Greenberg P D, Roederer M and Davis M M, Characterization of circulating T cells specific for tumor-associated antigens in melanoma patients. Nat Med 1999. 5: 677-685.
Lim A, Baron V, Ferradini L, Bonneville M, Kourilsky P and Pannetier C, Combination of MHC-peptide multimer-based T cell sorting with the Immunoscope permits sensitive ex vivo quantitation and follow-up of human CD8+ T cell immune responses. J Immunol Methods 2002. 261: 177-194.
Lu Z, Yuan L, Zhou X, Sotomayor E, Levitsky H I and Pardoll D M, CD40-independent pathways of T cell help for priming of CD8(+) cytotoxic T lymphocytes. J Exp Med 2000. 191: 541-550.
Lyons A B and Parish C R, Determination of lymphocyte division by flow cytometry. J Immunol Methods 1994. 171: 131-137.
Lyons A B, Analysing cell division in vivo and in vitro using flow cytometric measurement of CFSE dye dilution. J Immunol Methods 2000. 243: 147-154.
Mailliard R B, Egawa S, Cai Q, Kalinska A, Bykovskaya S N, Lotze M T, Kapsenberg M L, Storkus W J and Kalinski P, Complementary dendritic cell-activating function of CD8+ and CD4+ T cells: helper role of CD8+ T cells in the development of T helper type 1 responses. J Exp Med 2002. 195: 473-483.
Manz R, Assenmacher M, Pfluger E, Miltenyi S and Radbruch A, Analysis and sorting of live cells according to secreted molecules, relocated to a cell-surface affinity matrix. Proc Natl Acad Sci USA 1995. 92: 1921-1925.
Mason D, A very high level of crossreactivity is an essential feature of the T-cell receptor. Immunol Today 1998. 19: 395-404.
Mathioudakis G, Coder D and Fefer A, Multiparameter cytokine-specific affinity), matrix assay for the determination of frequencies and phenotype of antigen-reactive T cells. J Immunol Methods 2002. 260: 37-42.
Mentzer S J, Guyre P M, Burakoff S J and Faller D V, Spontaneous aggregation as a mechanism for human monocyte purification. Cell Immunol 101 1986. 312-319.
Migueles S A, Laborico A C, Shupert W L, Sabbaghian M S, Rabin R, Hallahan C W, Baarle D V, Kostense S, Miedema F, McLaughlin M, Ehler L, Metcalf J, Liu S and Connors M, HIV-specific CD8+ T cell proliferation is coupled to perforin expression and is maintained in nonprogressors. Nature Immunology 2002. 3, 1061-1068.
Mintern J D, Davey G M, Belz G T, Carbone F R and Heath W R, Cutting Edge: Precursor frequency affects the helper dependence of cytotoxic T cells. J Immunol 2002. 168: 977-980.
Murali-Krishna K, Altman J D, Suresh M, Sourdive D J, Zajac A J, Miller J D, Slansky J and Ahmed R, Counting antigen-specific CD8 T cells: a reevaluation of bystander activation during viral infection. Immunity 1998. 8: 177-187.
Mylin L M, Schell T D, Roberts D, Epler M, Boesteanu E, Collins E J, Frelinger J A, Joyce S and Tevetha S S, J Virol, 2000. 74-6922-6934.
Nestle F O, Alijagic S, Gilliet M, Sun Y, Grabbe S, Dummer R., Burg G, Schadendorf D, Vaccination of melanoma patients with peptide- or tumor lysate-pulsed dendritic cells, Nat Med. 1998. 4: 328-32.
Novak E J, Liu A W, Nepom G T and Kwok W W, MHC class II tetramers identify peptide-specific human CD4⁺ T cells proliferating in response to influenza A antigen, J Clin Invest, 1999. 104:R63-67.
Pala P, Hussell T and Openshaw P J, Flow cytometric measurement of intracellular cytokines J Immunol Methods 2000. 243: 107-124.
Pittet M J, Speiser D E, Valmori D, Rimoldi D, Lienard D, Lejeune F, Cerottini J C and Romero P, Ex vivo analysis of tumor antigen specific CD8+ T cell responses using MHC/peptide tetramers in cancer patients. Int Immunopharmacol 2001. 1: 1235-1247.
Pittet M J, Valmori D, Dunbar P R, Speiser D E, Lienard D, Lejeune F. Fleischhauer K, Cerundolo V, Cerottini J C and Romero P, High frequencies of naive Melan-A/MART-1-specific CD8(+) T cells in a large proportion of human histocompatibility leukocyte antigen (HLA)-A2 individuals. J Exp Med 1999. 190: 705-715.
Ponsaerts P, Van Tendeloo V F, Cools N, Van Driessche A, Lardon F, Nijs G, Lenjou M, Mertens G, Van Broeckhoven C, Van Bockstaele D R, Berneman Z N, mRNA-electroporated mature dendritic cells retain transgene expression, phenotypical properties and stimulatory capacity after cryopreservation. Leukemia. 2002 16: 1324-30.
Ramachandra L, Noss E, Boom W H and Harding C V, Cell Phagocytic processing of antigens for presentation by class II major histocompatibility complex molecules. Microbiol. 1999. 1:205-14.
Reis e Sousa C, Yap G, Schulz O, Rogers N, Schito M, Aliberti J, Hieny S and Sher A, Paralysis of dendritic cell IL-12 production by microbial products prevents infection-induced immunopathology. Immunity 1999. 11: 637-647.
Ridge J P, Di Rosa F and Matzinger P, A conditioned dendritic cell can be a temporal bridge between a CD4+ T-helper and a T-killer cell. Nature 1998. 393: 474-478.
Rubio-Godoy V, Dutoit V, Rimoldi D, Lienard D, Lejeune F, Speiser D, Guillaume P, Cerottini J C, Romero P and Valmori D, Discrepancy between ELISPOT IFN-gamma secretion and binding of A2/peptide multimers to TCR reveals interclonal dissociation of CTL effector function from TCR-peptide/MHC complexes half-life. Proc Natl Acad Sci USA 2001. 98, 10302-7.
Sallusto F, Lenig D, Forster R, Lipp M and Lanzavecchia A, Two subsets of memory T lymphocytes with distinct honing potentials and effector functions. Nature 1999. 401: 708-712. Sambrook et al., MOLECULAR CLONING, A LABORATORY MANUAL, Cold Spring Harbor Press, Cold Spring Harbor, N.Y. 1989.
Sandberg J K, Fast N M and Nixon D F., Functional heterogeneity of cytokines and cytolytic effector molecules in human CD8+ T lymphocytes. J Immunol 2001. 167: 181-187.
Song, H K, Noorchashm, H, Lieu, Y K, Rostami, S, Greeley, S A, Barker, C F, a Naji, A, Cutting edge: alloimmune responses against major and minor histocompatibility antigens: distinct division kinetics and requirement for CD28 co-stimulation. J Immunol 1999. 162: 2467-2471.
Stewart & Young, SOLID PHASE PEPTIDE SYNTHESIS, 2D. ED., Pierce Chemical Co., 1984.
Strome S E, Voss S, Wilcox R, Wakefield T L, Tamada K, Flies D, Chapoval A, Lu J, Kasperbauer J L, Padley D, Vile R, Gastineau D, Wettstein P, Chen L., Strategies for antigen loading of dendritic cells to enhance the antitumor immune response. Cancer Res. 2002. 62: 1884-9.
Valmori D, Fonteneau J F, Lizana C M, Gervois N, Lienard D, Rimoldi D, Jongeneel V, Jotereau F, Cerottini J C and Romero P, Enhanced generation of specific tumor-reactive CTL in vitro by selected Melan-A/MART-1 immunodominant peptide analogues. J Immunol 1998. 160: 1750-1758.
van Stipdonk M J, Lemmens E E and Schoenberger S P, Naive CTLs require a single brief period of antigenic stimulation for clonal expansion and differentiation Nat Immunol 2001. 2: 423-429.
Veiga-Fernandes H, Walter U, Bourgeois C, McLean A and Rocha B, Response of naive and memory CD8+ T cells to antigen stimulation in vivo. Nat Immunol 2000. 1: 47-53.
Vieira P L, de Jong E C, Wierenga E A, Kapsenberg M L and Kalinski P, Development of Th1-inducing capacity in myeloid dendritic cells requires environmental instruction J Immunol 2000. 164: 4507-4512.
Wallace P K, Palmer L D, Perry-Lalley D, Bolton E S, Alexander R B, Horan P K, Yang J C and Muirhead K A, Mechanisms of adoptive immunotherapy: improved methods for in vivo tracking of tumor-infiltrating lymphocytes and lymphokine-activated killer cells. Cancer Res: 1993. 2358-2367.
Wan Y, Lu L, Bramson J L, Baral S, Zhu Q, Pilon A and Dayball K, Dendritic cell-derived IL-12 is not required for the generation of cytotoxic, IFN-gamma-secreting, CD8(+) CTL in vivo J Immunol 2001. 167: 5027-5033.
Wang B, Norbury C C Greenwood R, Bennink J R, Yewdell J W and Frelinger J A, Multiple paths for activation of naive CD8+ T cells: CD4-independent help. J Immunol 2001. 167: 1283-1289.
Wells A D, Gudmundsdottir H and Turka L A, Following the fate of individual T cells throughout activation and clonal expansion Signals from T cell receptor and CD28 differentially regulate the induction and duration of a proliferative response. J Clin Invest 1997. 100: 3173-3183.
Wolfers J, Lozier A, Raposo G, Regnault A, Thery C, Masurier C, Flament C, Pouzieux S, Faure F, Tursz T, Angevin E, Amigorena S, Zitvogel L, Tumor-derived exosomes are a source of shared tumor rejection antigens for CTL cross-priming. Nat Med. 2001, 7: 297-303.
Yewdell J, To DRiP or not to DRiP: generating peptide ligands for MHC class I molecules from biosynthesized proteins. Mol Immunol. 2002. 39:139-46.
Zarling A L, Johnson J G, Hoffman R W and Lee D R, Induction of primary human CD8+ T lymphocyte responses in vitro using dendritic cells J Immunol 1999. 162: 5197-5204.
Zippelius A, Pittet M J, Batard P, Rufer N, de Smedt M, Guillaume P, Ellefsen K, Valmori D, Liénard D, Plum J, MacDonald H R, Speiser D E, Cerottini, J C and Romero P, Thymic selection generates a large T cell pool recognizing a self-peptide in humans. J Exp Med 2002. 195: 485-494. Allsopp, C. E., Nicholls, S. J. and Langhorne, J., A flow cytometry method to assess antigen-specific proliferative responses of different subpopulations of fresh and cryopreserved human peripheral blood mononuclear cells. J Immunol Methods 1998. 214: 175-186.

1-49. (canceled)

50. A method to characterize a T-cell response of a final population of T-lymphocytes resulting from the co-incubation of an initial population of T lymphocytes with a composition of purified antigen-presenting cells (APCs), said method comprising the following steps:
1. the simultaneous measure on a single cell basis of at least two parameters:
i. the first parameter being necessarily proliferation of T lymphocytes,
ii. the second parameter being necessarily chosen among the group consisting of presence of a T cell antigen receptor on the surface of T lymphocytes and presence of at least one biological molecule produced by T lymphocytes,
and the attribution of a positive or a negative value to each of these parameters,
2. the classification of the final T-lymphocytes population into 2ⁿ different subsets of T lymphocytes, n being the number of parameters used for the measure, each subset being characterized by a positive or a negative value respectively to each parameter, and the determination of the proportion of T lymphocytes present in each subset with respect to the number of T lymphocytes in the final population, with said proportion being characteristic of the T-cell response.

51. The method according to claim 1, further comprising evaluating said parameters as part of
a potency assay for a composition of purified APCs or
a method to evaluate the effect, on a T-cell response, of one or more cytokines secreted by a composition of purified APCs or
a method to evaluate the effect, on a T-cell response, of one or more surface determinant markers present on T-cells or
a batch release assay of a composition of purified APCs or
an inclusion criterion for a patient wherein the composition of purified APCs originating from said patient have the ability to induce a proliferation of T lymphocytes, resulting in a proliferation index at least greater than 2, more preferably of at least 5, more preferably of at least 10, more preferably of at least 15, more preferably of at least 20, more preferably of at least 30, more preferably of at least 50 or
an antigen selecting assay wherein the antigen to be tested is loaded on purified APCs and the T lymphocyte response triggered by the co-incubation with said loaded purified APCs is compared to the T lymphocyte response induced by a composition of purified APCs loaded with reference antigen or
method to define a standard control T-cell response of T-lymphocytes comprising:
the co-incubation of an initial population of T-lymphocytes with different compositions of purified APCs presenting different concentrations of an antigen or of an antigen fragment of interest or of a reference antigen or of a fragment of reference antigen and,
the determination of the variation of the degree of proliferation of said T-lymphocytes measured for each composition of purified APCs according the quantity of said antigen or said fragment of antigen of interest or said reference antigen or said fragment of reference antigen.

52. The method according to claim 51, wherein the step (1) of said method comprises the measure of an additional parameter being the presence or not of at least one surface determinant marker on T lymphocytes, different from T cell antigen receptors.

53. The method according to claim 53, wherein purified antigen-presenting cells are loaded with at least one antigen or fragment of antigen.

54. The method according to claim 53, wherein the T cell antigen receptor on the surface of T lymphocytes is specific or not for an antigen or fragment of antigen loaded on said purified APCs.

55. The method according to claim 54, wherein the T cell antigen receptor on the surface of T lymphocytes is specific for an antigen or of a fragment of antigen loaded on said purified APCs.

56. The method according to claim 51, wherein said method comprises a third step of determination, from the proportion of T lymphocytes in each of the different subset present in the final population, of the proportion of T lymphocytes in each corresponding subset present in the initial population with respect to the number of T lymphocytes in the initial population.

57. The method according to claim 56, wherein the determination of the proportion of T lymphocytes present in the initial population of T-lymphocytes loaded with a fluorescent probe allowing the measure of proliferation is made according to following steps:
(i) marking n minus 1 parameters, the parameter corresponding to the proliferation being previously marked, with fluorescent probes specific for each of the n minus 1 parameters,
(ii) gating T-lymphocytes in the final population of T lymphocytes according to the fluorescence of the n minus 1 chosen parameters, the measure of proliferation being excluded at this step, the value of which define lymphocytes subsets of interest,
(iii) building a fluorescent curve by recording the fluorescence intensity of the probe used to measure proliferation of the T-lymphocytes gated at step (ii),
(iv) possibly building a fluorescent curve by recording the fluorescence intensity of the probe used to measure proliferation from either:
(iva) T-lymphocytes present in a lymphocytes subset defined by gating lymphocytes in the final population of T lymphocytes according to the absence of fluorescence of the n minus 1 chosen parameters or,
(ivb) T-lymphocytes present in a sample of T-lymphocytes of the initial population not co-incubated with purified APCs,
(v) determining intensity of fluorescence of non-proliferating lymphocytes by analyzing the distribution of fluorescence of the fluorescent curve built at step (iii), or possibly at step (iv), the non-proliferating lymphocytes corresponding to the maximal value of fluorescence,
(vi) deriving, from the fluorescence curve recorded at step (iii), Gaussian curves centered on successive half intensity values derived from the maximal intensity of fluorescence determined from non-proliferating T-lymphocytes at step (iii) or at step (iv), to obtain Aₖ which is the proportion of cells in division k at the time of the measure of the proliferation,
(vii) determining the proportion of T-lymphocytes (PF=precursor frequency) in the initial population that have proliferated in order to give the proportion of T lymphocytes present in the selected subset (step ii) using the formula:
$$PF = ( \begin{matrix}k = n \\ \sum {A}_{k} / {2}^{k} \\ k = 2\end{matrix} ) / ( \begin{matrix}k = n \\ \sum {A}_{k} / {2}^{k} \\ k = 0\end{matrix} )$$
wherein PF is precursor frequency in the initial population, Aₖ is the proportion of cells in division k at the time of the measure of the proliferation, k=0 for initial population of T lymphocytes, and cells having undergone 2 to n divisions having been classified as proliferating T lymphocytes,
(viii) determining the percentage of non-proliferating T-lymphocytes from the percentage of T-lymphocytes that have not proliferated and that are present in the final population of T-lymphocytes and half the percentage of T-lymphocytes that had undergone only one cell division,
(ix) applying the percent of non-proliferating T-lymphocytes to the number of gated T-lymphocytes in the data file to give the absolute number of T-lymphocytes in the corresponding subset before culture that will not proliferate according to the formula,
number_{non-proliferating cells in the initial population}[(proportion_{cells that have not proliferated and that are present in the sample at the end of the experiment})+(0.5*proportion_{cells that have divided only once and that are present in the sample at the end of the experiment})]*[number_{gated cells in data file}])
(x) determining the absolute number of T-lymphocytes in the corresponding subset destined to divide by knowing the number of T-lymphocytes that was not destined to divide and the number of precursor cells of proliferating T-lymphocytes according to the formula,
number_{proliferating cells in the initial population}=[(PF_{proliferating cells})*(number_{non-proliferating cells in the initial population})]/[1−PF_{proliferating cells}],
(xi) reiterating step (i) to step (viii) to each T lymphocytes subsets determined according to the n parameters used for the measure,
(xii) summation of number of cells in the 2ⁿ subsets in order to express the number of T-lymphocytes present in the initial population of T-lymphocytes as a percentage of the total initial population before co-incubation.

58. The method according to claim 51, wherein purified APCs are purified monocyte-derived antigen presenting cells.

59. The method according to claim 51, wherein purified APCs are purified immature, maturing or mature dendritic cells, monocytes or macrophages.

60. The method according to claim 53, wherein purified APCs are loaded with at least one antigen or a fragment of antigen which is an antigen of tumoral or infectious origin.

61. The method according to claim 60, wherein said antigen is comprised in the group consisting of: EBV, CMV, HBV, p53, tetanus toxin, Melan-A/MART-1, MAGE-3, MAGE-2, PSA, PSMA, PAP, HSP70, CEA, Ep-CAM, MUC1, MUC2, HER2/neu, M1 protein from the influenza virus, or peptides derived from these proteinic antigens.

62. The method according to claim 51, wherein co-incubation of purified APCs and T lymphocytes lasts for a time sufficient to allow at least 1 division of T lymphocytes, preferably 5 divisions.

63. The method according to claim 62, wherein co-incubation lasts from 1 to 10 days, and more preferably 4 to 10 days.

64. The method according to claim 51, wherein co-incubation comprises a step of adding a T lymphocyte stimulating agent at the end of the co-incubation period.

65. The method according to claim 51, wherein proliferation of T lymphocytes is measured by using a probe loaded into T lymphocytes before or concomitantly to the step of co-incubation, said probe being substantially equally distributed between dividing T-lymphocytes during cell division of T lymphocytes of the initial population.

66. The method according to claim 65, wherein the probe loaded before the co-incubation step is fluorescent and distributed in the cell membrane or inside the cytosol of T-lymphocytes.

67. The method according to claim 65, wherein the probe loaded into the T-lymphocytes concomitantly to the co-incubation step is a base analog that integrates into the DNA, such as BrdU.

68. The method according to claim 51, wherein the presence of a specific T cell antigen receptor on the surface of T lymphocytes is detected by using a detectable molecule having the ability to specifically bind to said T cell antigen receptor, such as a fluorescent tetramer.

69. The method according to claim 51, wherein the T cell antigen receptors on the surface of T lymphocytes are specific for an antigen from tumor or infectious origin.

70. The method according to claim 69, wherein said antigen is chosen among the group consisting of: EBV, CMV, HBV, M1 protein from the influenza virus, p53, tetanus toxin, Melan-A MART-1, MAGE-3, MAGE-2, PSA, PSMA, PAP, HSP70, CEA, Ep-CAM, MUC1, MUC2, HER2/neu, or peptides derived from these proteinic antigens.

71. The method according to claim 51, wherein the biological molecule whose presence is detected in final population of T lymphocytes, is a cytokine or a chemokine or an enzyme.

72. The method according to claim 71, wherein the cytokine is chosen among the group consisting of: IFN-γ, IL-2, IL-4, IL-5, IL-10.

73. The method according to claim 71, wherein the enzyme is chosen among the group consisting of perforine and granzyme.

74. The method according to claim 71, wherein the chemokine is chosen among the group consisting of a ligand for CCR5 and a ligand for CCR7.

75. The method according to claim 52, wherein the surface determinant marker of T-lymphocytes is chosen among the group consisting of CD4, CD8, CD28, CD69, CTLA-4, CD45-RA, CD45-RO, CD62-L.

76. The method according to claim 51, wherein the proportion of T lymphocytes in the different subsets of T lymphocytes in the final population and the proportion of T lymphocytes in the different corresponding subsets of T lymphocytes in the initial population are used to determine a proliferation index (PI) for each subset of T lymphocytes using the formula:
$$PI = ( \begin{matrix}k = n \\ \sum {A}_{k} \\ k = 0\end{matrix} ) / ( \begin{matrix}k = n \\ \sum {A}_{k} / {2}^{k} \\ k = 0\end{matrix} )$$
wherein Aₖ is the proportion of cells in division k

77. The method according to claim 51, wherein said method is used as a potency assay for a composition of purified APCs.

78. The method according to claim 77, wherein the purified APCs ability to activate T lymphocytes is characterized by the determination of the proliferation index and/or by the proportion of T lymphocytes precursors present in the initial population.

79. The method according to claim 28, wherein purified APCs are characterized by their ability to induce proliferation of T lymphocytes, positive at least for the proliferation parameter, resulting in a proliferation index of at least 2, more preferably of at least 5, more preferably of at least 10, more preferably of at least 15, more preferably of at least 20, more preferably of at least 30, more preferably of at least 50.

80. The method according to claim 79, wherein purified APCs are characterized by their ability to induce a proliferation of T lymphocytes, positive at least for the proliferation parameter, resulting in a proliferation index ranging between 2 and 200, more particularly from 15 to 70, more particularly from 20 to 60, more particularly from 30 to 40 and more particularly from 20 to 200.

81. The method according to claim 51, wherein said method is used as a method to evaluate the effect, on a T-cell response, of one or more cytokines secreted by a composition of purified APCs.

82. The method according to claim 81, wherein the co-incubation of an initial population of T lymphocytes with a composition of purified antigen-presenting cells (APCs) takes place in the presence of an antibody able to bind specifically to a cytokine that is produced during co-incubation.

83. The method according to claim 82, wherein the cytokine is chosen among the group consisting of: IL-2, IL-10, IL-12, IL-15, IL-18, IL-23, TNF-α, TGF-β.

84. The method according to claim 51, wherein said method is used as a method to evaluate the effect, on a T-cell response, of one or more surface determinants markers present on T-cells.

85. The method according to claim 84, wherein the co-incubation of T-lymphocytes and purified APCs takes place in the presence of an antibody, or an antagonist, able to bind specifically to a surface determinant marker of T-lymphocytes.

86. The method according to claim 85, wherein the surface determinant marker is CD4, CD8, CD28, CTLA-4, B7, LFA-10, OX40-ligand, ICAM-1, 4-1BBL, DC-SIGN or MHC-II.

87. The method according to claim 51, wherein said method is used as a batch release assay of a composition of purified APCs.

88. The method according to claim 87, wherein purified APCs are characterized by the different percentages of T lymphocytes secreting a cytokine during the co-incubation.

89. The method according to claim 51, wherein said method is used as an inclusion criterion for a patient wherein the composition of purified APCs originating from said patient have the ability to induce a proliferation of T lymphocytes, resulting in a proliferation index at least greater than 2, more preferably of at least 5, more preferably of at least 10, more preferably of at least 15, more preferably of at least 20, more preferably of at least 30, more preferably of at least 50.

90. The method according to claim 54, wherein said method is used as an antigen selecting assay wherein the antigen to be tested is loaded on purified APCs and the T lymphocyte response triggered by the co-incubation with said loaded purified APCs is compared to the T lymphocyte response induced by a composition of purified APCs loaded with a reference antigen.

91. The method according to claim 90, wherein the reference antigen is chosen among the group consisting of: tetanus toxin, Melan-A, Flu peptide, PSA, and HIV.

92. The method according to claim 51, wherein said method is used to define a standard control T-cell response of T-lymphocytes comprising:
the co-incubation of an initial population of T-lymphocytes with different compositions of purified APCs presenting different concentrations of an antigen or of an antigen fragment of interest or of a reference antigen or of a fragment of reference antigen and,
the determination of the variation of the degree of proliferation of said T-lymphocytes measured for each composition of purified APCs according the quantity of said antigen or said fragment of antigen of interest or said reference antigen or said fragment of reference antigen.

93. The method according to claim 92, to evaluate the efficiency of a process for loading an antigen, or a fragment of antigen, into purified APCs wherein the efficiency of the said process is evaluated by comparing:
a first response being a T-cell response of a final population of T-lymphocytes resulting from the co-incubation of an initial population of T-lymphocytes with a composition of purified APCs loaded with an antigen or a fragment of antigen, according to the process to be tested and,
a second response being a standard control T-cell response of T-lymphocytes resulting from the co-incubation of an initial population of T-lymphocytes with different compositions of purified APCs loaded with different concentrations of said antigen or said fragment of antigen, or of a reference antigen, or of a fragment of reference antigen according to the process of reference,
deducing from said comparison between said first and said second responses the difference of efficiency between the process to be tested and the process of reference.

94. The method according to claim 93, wherein the reference process is chosen among the group consisting of: fusion, electroporation, incubation, loading with liposomes, loading with virosomes, loading with exosomes or genetic engineering of antigen-presenting cells.

95. The method according to claim 92, to evaluate an impact of a method of an antigen preparation on the ability of an antigen-presenting cell to present antigen to T lymphocyte wherein the said method of preparation of antigen is evaluated by comparing:
a first response being a T-cell response of a final population of T-lymphocytes resulting from the co-incubation of an initial population of T-lymphocytes with a composition of purified APCs loaded with an antigen or a fragment of antigen, prepared according to the method to be tested,
a second response being a standard control T-cell response of T-lymphocytes resulting from the co-incubation of an initial population of T-lymphocytes with different compositions of purified APCs loaded with different concentrations of said antigen or said fragment of antigen, or of a reference antigen, or of a fragment of reference antigen, prepared according to a method of reference,
deducing from said first and said second responses the impact of said method of antigen preparation to be tested on the ability of an antigen-presenting cell to present antigen to T lymphocyte.

96. The method according to claim 92 to evaluate stability of a presentation of an antigen (or fragment of antigen) by purified APCs wherein the said stability is evaluated by comparing:
a first response being a T-cell response of a final population of T-lymphocytes resulting from the co-incubation of an initial population of T-lymphocytes with different compositions of purified APCs loaded with said antigen (or fragment of antigen) said compositions of purified APCs being previously incubated in a medium not initially containing said antigen for different period of time and,
a second response being a standard control T-cell response of T-lymphocytes resulting from the co-incubation of an initial population of T-lymphocytes with composition of purified APCs loaded with an antigen or a fragment of antigen, or a reference antigen, or a fragment of reference antigen said compositions of purified APCs being not previously incubated in a medium not initially containing said antigen or said fragment of antigen, or a reference antigen, or a fragment of reference antigen,
deducing from the first and the second responses the stability of a presentation of said antigen (or fragment of antigen) by purified APCs.

97. The method according to claim 92, wherein the initial population of T lymphocytes is a clonal population or a cell line of T lymphocytes that is specific to the antigen, or fragment of antigen, presented in the context of MHC.

98. The method according to claim 92, wherein the initial population of T lymphocytes is an initial naïve population of T lymphocytes, said initial naive population of T lymphocytes being substantially the same for obtaining a standard control T-cell response of T-lymphocytes and a T-cell response to be compared to said standard control T-cell response of T-lymphocytes.
